(19) United States
(12) Patent Application Publication
(10) Pub. No.: US 2013/0178386 A1
(43) Pub. Date: Jul. 11, 2013

(54) PLATELET BIOMARKERS FOR THE DETECTION OF DISEASE

(71) Applicant: THE NEWMAN-LAKKA CANCER FOUNDATION, Scottsdale, AZ (US)

(72) Inventors: Judah Folkman, Brookline, MA (US); Giannoula Klement, Boston, MA (US)

(73) Assignee: THE NEWMAN-LAKKA CANCER FOUNDATION, Scottsdale, AZ (US)

(21) Appl. No.: 13/706,483

(22) Filed: Dec. 6, 2012

Related U.S. Application Data
(63) Continuation of application No. 11/304,384, filed on Dec. 15, 2005, now abandoned.
Application No. 11/304,384 is a continuation of application No. PCT/US05/14210, filed on Apr. 26, 2005.
(60) Provisional application No. 60/565,286, filed on Apr. 26, 2004.
(60) Provisional application No. 60/598,387, filed on Aug. 2, 2004.
(60) Provisional application No. 60/609,692, filed on Sep. 13, 2004.
(60) Provisional application No. 60/633,027, filed on Dec. 3, 2004.
(60) Provisional application No. 60/633,613, filed on Dec. 6, 2004.

(51) Int. Cl. G01N 33/68

(52) U.S. Cl. CPC G01N 33/6893
USPC 506/9

(57) ABSTRACT

The present inventors have surprisingly discovered that platelets sequester angiogenic regulators and prevent their degradation. Thus, by analyzing levels of angiogenic regulators in platelets, it is now possible to detect angiogenic activity, even at an early stage. By monitoring for changes in angiogenic activity, the presence of cancer or other angiogenic diseases or disorders can be predicted.

### CROSS REFERENCE TO RELATED APPLICATIONS

This application is a continuation application of U.S. patent application Ser. No. 11/304,384 filed on Dec. 15, 2005, which is a continuation of International Application No. PCT/US05/14210 filed Apr. 26, 2005, which claims benefit under 35 U.S.C. §119(e) of U.S. Provisional Application No. 60/565,286, filed Apr. 26, 2004, U.S. Provisional Application No. 60/598,387 filed Aug. 2, 2004, U.S. Provisional Application No. 60/609,692 filed Sep. 13, 2004, U.S. Provisional Application No. 60/633,027 filed Dec. 3, 2004, and U.S. Provisional Application No. 60/633,613 filed Dec. 6, 2004, the contents of each of which are herein incorporated by reference in their entireties.

### BACKGROUND OF THE INVENTION

Angiogenesis is a process of tissue vascularization that involves the growth of new developing blood vessels into a tissue, and is also referred to as neo-vascularization. Blood vessels are the means by which oxygen and nutrients are supplied to living tissues and waste products are removed from living tissue. When appropriate, angiogenesis is a critical biological process. For example, angiogenesis is essential in reproduction, development and wound repair. Conversely, inappropriate angiogenesis can have severe negative consequences. For example, it is only after solid tumors are vascularized as a result of angiogenesis that the tumors have a sufficient supply of oxygen and nutrients that permit it to grow rapidly and metastasize.

Angiogenesis-dependent diseases are those diseases which require or induce vascular growth. Such diseases represent a significant portion of all diseases for which medical treatment is sought, and include inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

In a recent review by Folkman, it was estimated that more than one-third of all women between the ages of 40 and 50 have in-situ tumors in their breasts. Such tumors lie dormant in the body and rarely, if ever, are diagnosed as breast cancer. It is believed that a similar phenomenon exists in men in regards to prostate cancer. In light of such data, cancer might be defined as having two distinct phases: (1) acquisition of mutations which transform normal cells into cancerous cells, and the formation of in-situ tumors; and (2) a switch to an angiogenic phenotype, whereby the in-situ tumor is supplied with new blood vessels, supporting rapid tumor growth and metastasis (Nature, Vol. 427, Feb. 26, 2004, p. 787). A method to detect a tumor before the angiogenic switch, i.e. at the time of formation of an in-situ tumor, is needed.

Angiogenesis is driven by a balance between different positive and negative effector molecules influencing the growth rate of capillaries. Various angiogenetic and anti-angiogenetic factors have been cloned to date and are known (Leung et al., Science. 246: 1306-9, 1989; Ueno et al., Biochem Biophys Acta. 1382: 17-22, 1998; Miyazono et al., Prog Growth Factor Res. 3: 207-17, 1991). Vascular endothelial growth factor (VEGF) and trombospondin-1 (TSP-1) are two of the most well studied. VEGF is an angiogenic factor as opposed to TSP-1, which functions as an anti-angiogenic molecule (Tuszynski et al., Bioes says. 18: 71-6, 1996; Dameron, et al. Science. 265: 1582-4, 1994). Normal vessel growth results by balanced and coordinated expression of these opposing factors. A switch from normal to uncontrolled vessel growth can occur by up-regulating angiogenesis stimulators or down-regulating angiogenesis inhibitors, suggesting that the angiogenetic process is tightly regulated by the oscillation between these opposing forces (Bouck et al., Adv Cancer Res. 69: 135-74, 1996). For example, in tumor tissues, the switch to an angiogenic phenotype occurs as a distinct step before progression to a neoplastic phenotype and is linked to epigenetic or genetic changes (Hanahan et al., Cell. 86: 353-64, 1996). In support of this theory, mRNA expression of VEGF is up-regulated in aggressive tumor cell lines expressing an activated ras oncogene (Rak et al., Neoplasia. 1: 23-30, 1999). Conversely, transcription of VEGF is down-regulated in these same tumor cell lines after disruption of the mutant ras allele, thus eliminating VEGF expression and rendering the cells incapable of tumor formation in vivo. (Stiegler et al., J Cell Physiol. 179: 233-6, 1999). The switch to an angiogenic phenotype has also been associated with the inactivation of the tumor suppressor gene p53 (Holmgren et al., Oncogene. 17: 819-24, 1998). Conversely, cell lines that are p16 deleted revert to an anti-angiogenic phenotype upon the restoration of wild type cyclin dependent kinase (cdk) inhibitor p16 (Harada et al., Cancer Research. 59: 3783-3789, 1999).

The majority of cancers are detected using techniques such as MRIs, biomarkers, e.g., PSA, mammography, palpation, and tissue biopsy. Using such methods, most cancers are discovered only after they are either considerably developed or metastasized. Therefore, the opportunity for any early cure is often missed. This is in part due to the low accuracy of conventional diagnostic methods and the need for expensive equipments, such as NMRS, tomographs, etc., which can be a financial burden for patients. Furthermore, patients must be hospitalized to receive accurate assays, such as tissue biopsy. Thus, conventional diagnostic methods are not optimal for the early diagnosis of cancer and none of the aforementioned techniques lends itself to rapid or simple procedure for early detection of cancer.

The angiogenic process is believed to begin with the degradation of the basement membrane by proteases secreted from endothelial cells (EC) activated by mitogens such as vascular endothelial growth factor (VEGF), basic fibroblast growth factor (bFGF), interleukin-8 (IL-8), placenta-like growth factor (PLGF), transforming growth factor-β (TGF-β), and others. The cells migrate and proliferate, leading to the formation of solid endothelial cell sprouts into the stromal space, then, vascular loops are formed and capillary tubes develop with formation of tight junctions and deposition of new basement membrane. Angiogenesis may also involve the downregulation of angiogenesis suppressor proteins, such as thrombospondin.

The therapeutic implications of angiogenic growth factors were first described by Folkman and colleagues over three decades ago (Folkman, N. Engl. J. Med., 285:1182-1186 (1971). Abnormal angiogenesis occurs when the body loses at least some control of angiogenesis, resulting in either excessive or insufficient blood vessel growth. For instance, conditions such as ulcers, strokes, and heart attacks may result from the absence of angiogenesis normally required for natural healing. In contrast, excessive blood vessel proliferation can result in tumor growth, tumor spread, premature or diabetic retinopathy, psoriasis and rheumatoid arthritis.

Angiogenic regulators have a very short half life, for example, the half life of the native VEGF in the plasma is about three minutes. Therefore, current methods of measuring angiogenic growth factor levels to detect such regulators do not provide a reliable indication of angiogenic activity.

A method for the early detection of cancer and other angiogenic diseases and disorders is highly desirable.

### SUMMARY

The present inventors have surprisingly discovered that platelets sequester angiogenic regulators and prevent their degradation. Thus, by analyzing levels of angiogenic regulators in platelets, it is now possible to measure angiogenic activity. By monitoring for changes in angiogenic activity, the presence of cancer or other angiogenic diseases or disorders can be predicted.

Accordingly, the present invention provides a novel method for the detection of cancer in an individual. Preferably, the cancer is detected early. In a preferred embodiment, platelets are isolated from an individual (a patient) at a first time point. The platelets are analyzed for the level of at least one angiogenic regulator. The angiogenic regulator may be a positive or negative angiogenic regulator. At a second, later time point, platelets are isolated from the patient and analyzed for the level of the angiogenic regulator. Next, the level or levels of angiogenic regulators from the platelets of the first sample are compared to the levels of angiogenic regulators from the platelets of the second sample. An increase in the level of at least one positive angiogenic regulator in the platelets from the second sample, compared to the level of that positive angiogenic regulator in the first sample is indicative of cancer or other angiogenic disease or disorder. Alternatively, a decrease in the level of at lease one negative angiogenic regulator is the platelets from the second sample, compared to the level of that negative angiogenic regulator in the first sample is indicative of cancer or other angiogenic disease or disorder. In a preferred embodiment, platelets are isolated from a blood sample. Preferably, more than one angiogenic regulator is measured.

Positive angiogenic regulators include, but are not limited to, VEGF-A (VPC), VEGF-C, bFGF, HGF, angiopoietin-1, PDGF, EGF, IGF-1, IGF BP-3, BDNF, matrix metaloproteinases (MMPs), vitronectin, fibronectin, fibrinogen, heparanase, and sphingosine-1 PO₄.

Negative angiogenic regulators include, but are not limited to, PF-4, thrombospondin-1 & 2, NK1, NK2, NK3 fragments of HGF, TGF-beta-1, plasminogen (angiostatin), plasminogen activator inhibitor 1, alpha-2 antiplasmin and fragments thereof, alpha-2 macroglobulin, tissue inhibitors of metaloproteinases (TIMPs), beta-thromboglobulin, endostatin, tumstatin, BDNF (brain derived neurotrophic factor) and soluble VEGFR2.

Methods for analyzing positive or negative angiogenic regulators include, for example, protein array, an ELISA, a Western blot, surface enhanced laser desorption ionization spectroscopy, or Mass Spectrometry.

In one embodiment, the individuals have a genetic predisposition to cancer. The predisposition may be a mutation in a tumor suppressor gene. The tumor suppressor gene may include, for example, BRCA1, BRCA2, p53, p10, LKB1, MSH2 and WT1.

In another embodiment, the individuals has been previously treated for cancer. Alternatively, the patient is believed to be a healthy disease-free individual.

In a preferred embodiment, the isolation of blood at the second time point occurs at least one month after the first isolation. However, the second time point can be 2 months, 6 months, 10 months, or greater than one year after the first isolation.

The cancer to be detected and treated using the present methods include, but are not limited to, gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer, bladder cancer, hemangioblastomas, neuroblastomas, carcinomas, sarcomas, leukemia, lymphoma and myelomas.

In one embodiment of the present invention, a method for treating a patient affected with an angiogenic disease or disorder, e.g. cancer, is described. In such a method, a first platelet sample is isolated from an individual at a first time point and analyzed for levels of at least one positive or negative angiogenic regulator. A second platelet sample, isolated a later time point, is obtained from the individual and analyzed for the level of at least one positive or negative angiogenic regulator. Next, the levels of angiogenic regulators from the first platelet sample are compared to the levels of angiogenic regulators from the second platelet sample. A change in the level of the angiogenic regulator in the second sample, compared to that level in the first sample, is indicative of the presence of an angiogenic disease or disorder. After being diagnosed, a therapy is administered. An angiogenic therapy is preferred. The method of the present invention can be used to monitor the progress of the therapy. Using this method, it is not necessary to diagnose the exact disease or disorder. All that is required is that the therapy alter the platelet profile in a manner that indicates that the therapy is working. If it is found that a particular therapy is not effective, the therapy can be altered to provide for a more effective treatment.

Preferably, the anti-cancer therapy involves administering an angiogenesis inhibitor(s). Alternatively, the patient may be treated with chemotherapy, radiation, or surgical resection of the tumor, if large enough to detect. In another embodiment, the patient is administered a combination of above anti-cancer therapies.

Platelets may be utilized to deliver the anti-angiogenesis therapy. The inventors of the present invention have surprisingly discovered that platelets sequester and prevent the degradation of various angiogenic factors. In addition, the inventors have discovered that the platelets selectively release their loads at physiologically appropriate places, such as, for example, a tumor. Thus, once diagnosed, platelets may be loaded with an anti-cancer compound and delivered to the patient in need thereof. In such a method, the compound is selectively delivered to the site in need of therapy, i.e. a tumor.

Known angiogenesis inhibitors include, but are not limited to: direct angiogenesis inhibitors, Angiostatin, Bevacizumab (Avastin), Arresten, Canstatin, Caplostatin, Combretastatin, Endostatin, NM-3, Thrombospondin, Tumstatin, 2-methoxyestradiol, and Vitaxin; and indirect angiogenesis inhibitors: ZD1839 (Iressa), ZD6474, OSI774 (Tarceva), CI1033, PKI1666, IMC225 (Erbitux), PTK787, SU6668, SU11248, Herceptin, and IFN-α, CELEBREX® (Celecoxib), THALOMID® (Thalidomide), and IFN-α have also been recognized as angiogeneis inhibitors (Kerbel et al., Nature Reviews, Vol. 2, October 2002, pp. 727.

Also encompassed in the present invention is the treatment of angiogenic disease/disorders using “metronomic” chemotherapy. Metronomic chemotherapy involves the administration of low doses of chemotherapeutic agents, see Folkman, APIS 112:2004.

After diagnosis, the methods of the present invention allow for the evaluation of the treatment being employed. After treatment, the methods are useful in early detection of recurrence.

The methods of the present invention may also be used for the early detection of angiogenic diseases or disorders, including, for example, retinopathy, diabetic retinopathy, or macular degeneration. In addition, the methods of the present invention may be used for the early detection and treatment of chronic inflammatory disorders including, pyresis, pain, osteoarthritis, rheumatoid arthritis, migraine headache, neurodegenerative diseases (such as multiple sclerosis), Alzheimer's disease, osteoporosis, asthma, lupus and psoriasis.

In another embodiment of the present invention, a platelet profile is created that corresponds to a particular angiogenic disease or disorder, e.g. cancer. This platelet profile is also referred to as a standard or a register. In such an embodiment, a sample of platelet from an individual is isolated and analyzed for the presence or absence of particular angiogenic factors. A diagnosis is made by comparing this profile to the standard. For example, for the diagnosis of liposcarcoma, an angiogenic factor profile standard is created by analyzing patients with diagnosed liposarcoma. Using this standard for comparison, a platelet sample from an individual may be analyzed. A positive diagnosis is made if the individual (test) sample correlates to the standard. Likewise, this type of diagnostic can be utilized for any number of cancers, angiogenic diseases and disorders, inflammatory diseases or disorders, or vascular abnormalities.

Furthermore, the present invention provides a method for the monitoring of effectiveness of antiangiogenic therapies or for testing compounds for effectiveness in modulating levels of platelet angiogenic regulators in a host. In this embodiment, platelets from an individual (host or host animal) at a first time point are obtained and screened for the presence or absence of positive and negative angiogenic regulators. A platelet profile (or register) is created. Antiangiogenic therapy (or a test compound) is then administered to the individual (or host). At a second, later, time point, platelets from the same individual (or host) are obtained and screened for the presence or absence of positive and negative angiogenic regulators. A second platelet profile (or register) is obtained. The effectiveness of the antiangiogenic therapy (or test compound) is determined by comparing the first and the second platelet profile. A decrease in the levels of positive angiogenic regulator in the second sample compared to the first sample is indicative of an effective antiangiogenic therapy. Likewise, an increase in the level of negative angiogenic regulators in the second sample compared to the first sample is indicative of an effective antiangiogenic therapy. This embodiment allows for a relatively easy and quick method of analyzing the effectiveness of various therapies or for screening the effectiveness of test compounds. If it is found that a particular therapy is not effective, the therapy can be altered to provide for a more effective treatment.

Host animals include mammals e.g., mice and rats.

In this embodiment, the second sample of platelet from an individual (or host) may be obtained at anytime after the initiation of administration of an antiangiogenic therapy. For example, the second platelet sample may be obtained at about one week to about one month after the initiation of therapy. Alternatively, the second sample may be obtained at 2 months, 3 months, 6 months, or up to one year after the initiation of therapy.

Also encompassed in this embodiment, and other embodiments of the invention, is the analysis of more than two time points. For example, platelets may be analyzed at several time points during antiangiogenic therapy. In this manner, the effectiveness of the antiangiogenic therapy can be analyzed over time and changes in the treatment protocol may be analyzed.

Angiogenic regulators (both positive and negative) are known to those of skill in the art, but may also be proteins as yet unidentified or known proteins not identified as “angiogenic regulators”. As such, the methods of the present invention may identify known or unknown proteins as angiogenic regulators. Angiogenic regulators will also be referred to as biomarkers throughout and will be described in more detail below. The angiogenic regulators of the present invention include proteins, protein fragments such as cleaved proteins and fused proteins, such as bcr-ab1.

### BRIEF DESCRIPTION OF THE DRAWINGS

FIG. 1: In vitro loading of human platelets with Endostatin. Platelet rich plasma (PRP) was incubated with increasing concentrations of Endostatin for one hour, followed by isolation of platelets, washing and lysing to obtain pure protein extracts later submitted to SDS-PAGE. Standard Western blots using anti-human Endostatin, anti-human VEGF and anti-human bFGF reveals the negative correlation of increases in Endostatin with decreases in the intracellular content of both VEGF and bFGF.

FIG. 2: Selective displacement of platelet proteins in vitro by SDS-PAGE. The uptake of Endostatin into platelets pre-loaded with VEGF is not only full, unencumbered, and enhanced in comparison to the Endostatin pre-loading control (first lane of FIG. 2), but results in a full displacement of the pre-loaded VEGF (second lane of FIG. 2). In comparison, in the opposite experiment; i.e., the loading of VEGF into platelets pre-loaded with Endostatin, results in less complete displacement of the Endostatin.

FIG. 3: FIG. 3 shows counts per gram of tissues (×10⁵) in liver, Matrigel, spleen, kidney, plasma, and platelet fractions. The iodinated VEGF concentrated in platelets in many fold excess of its concentration in plasma.

FIG. 4: FIG. 4 shows profiles of PF4 (FIG. 4A), PDGF (FIG. 4B), and VEGF (FIG. 4C) in platelets and plasma from controls, non-angiogenic, and angiogenic samples. The results show the concentration of PF4, PDGF, and VEGF in the platelet samples.

FIG. 5: FIG. 5 shows profiles of bFGF (FIG. 5A), VEGF (FIG. 5B), PDGF (FIG. 5C), and ES (FIG. 5D) in platelets and plasma from liposarcoma bearing mice.

FIG. 6: The intracellular distribution of VEGF prior, during and post platelet activation using immunofluorescence is shown. In resting platelet, the majority of VEGF localizes to the intracellular, cytoplasmic portion of platelets (FIGS. 6A and 6B), moving to the ring form alignment of VEGF along the cell membrane (FIGS. 6C and 6D-insert), and then along the pseudopodia of the activated platelet (FIG. 6D). The pattern of activation induced platelet exocytosis is more suggestive of a direct exchange of the intracellular contents of platelets with the tissues than with the commonly adopted “release” of intracellular contents of platelets into the circulation.

FIG. 7: VEGF Localization in Resting and Activated Platelets. Double label immunofluorescence microscopy on fixed and permeabilized resting platelets was used to determine the intracellular localization of VEGF. Tubulin is concentrated in the marginal microtubule band in a resting platelet and this structure defines the platelet periphery (FIG. 7A). The anti-VEGF antibodies consistently labeled punctate, vesicle-like structures distributed throughout the platelet cytoplasm (FIGS. 7B and 7E). Double stain of activated platelets using fluorescently-labeled phaloidin and VEGF reveals persistent association of VEGF with the platelet even upon activation (FIGS. 7C and 7F). Platelet-shape change consistent with activation was clearly documented by the formation of lamelipodia and filopodia. The VEGF is seen both as punctate patterns in activated, spread platelets, but more VEGF was localized along filopodia and along the periphery of lamellipodia, than that remaining within the cytoplasm.

FIG. 8 shows the intracellular distribution of VEGF in platelets. FIG. 8A: platelets are stained with phalloidin. FIG. 8B: platelets are stained with anti-VEGF. FIG. 8C: overlay.

FIG. 9 shows the interaction of a platelet (right) with a megakaryocyte (left). The intracellular distribution of VEGF is shown by immunofluorescence.

FIG. 10 shows the intracellular distribution of VEGF (FIG. 10A), vWF (FIG. 10B) and an overlay (FIG. 10C) in platelets and megakaryocytes.

FIG. 11 shows a diagram of positive and negative angiogenic regulators within platelets.

FIG. 12 shows a diagram of the placement of matrigel (50 ng ¹²⁵I VEGF) in a mouse.

FIG. 13 shows a schematic of a vascularized human tumor, a non-angiogenic dormant cell, and an angiogenic growing cell.

FIG. 14 shows non-angiogenic vs angiogenic human liposarcoma in nude mice. Angiogenesis was analyzed by luciferase luminescence at 133 days.

FIG. 15 shows a protocol for platelet and plasma protein expression using SELDI-TOF.

FIG. 16 shows protein expression maps of extracts of platelets and plasma from SCID mice bearing non-angiogenic and angiogenic human lipsarcomas, 30 days after tumor implantation. VEGF is marked.

FIG. 17 shows protein expression maps of extracts of platelets and plasma from SCID mice bearing non-angiogenic and angiogenic human lipsarcomas, 30 days after tumor implantation. PF-4 is marked.

FIG. 18 shows protein expression maps of extracts of platelets and plasma from SCID mice bearing non-angiogenic and angiogenic human lipsarcomas, 30 days after tumor implantation. PDGF is marked.

FIG. 19 shows the time course of sequestration of bFGF in platelet of tumor-bearing mice. Only molecular weight of 1820 Daltons included.

FIG. 20A shows a mass spectrophotometric expression map of platelet extracts taken from control animals (grey lines) and animals implanted with dormant tumors (black lines). The numbers on the x-axis refer to the mass to charge ratios (m/z) of the observed particles and the heights of the curves correspond to the intensity of the observed peaks. The extracts used were obtained from fraction 2 of the initial anion exchange fractionation, as described in the Examples. Samples from this fraction were analyzed on the WCX2 ProteinChip array. CTAPIII and PF4 were identified to be up-regulated in tumor-bearing mice. FIG. 20B shows that CTAPIII and PF4 (arrows) were up-regulated in platelets of both dormant and angiogenic tumor-bearing mice, but not in plasma.

FIG. 21a shows a plot of the normalized CTAPIII peak intensity measured in extracts taken from the platelets of three groups of mice: controls, dormant (non-angiogenic) and angiogenic human liposarcoma tumors, respectively. FIG. 21B shows a plot of the normalized CTAPIII peak intensity measured in extracts taken from the plasma of three groups of mice: controls, dormant (non-angiogenic) and angiogenic human liposarcoma tumors, respectively.

FIG. 21C shows a plot of the normalized PF4 peak intensity in platelets of the same groups of mice as in 21A and 21B. FIG. 21D shows a plot of the normalized PF4 peak intensity in plasma of the same groups of mice as in 21A, 21B, and 21C.

FIG. 22A shows a plot of the normalized CTAPIII peak intensity in the platelets of tumor-bearing mice at 19 days, 32 days and 120 days of growth, indicating that platelet CTAP III levels increased over the time course studied, while FIG. 22B shows plasma CTAP III levels decreased, or did not change, over the same period.

FIG. 22C shows a plot of the normalized PF4 peak intensity in platelets of tumor-bearing mice at 19 days, 32 days and 120 days of growth, indicating that platelet PF4 levels increased over the time course studied, while FIG. 22D shows plasma PF4 levels decreased, or did not change, over the same period. The median±standard errors are shown for each group of peak intensities in FIG. 22.

FIG. 23a shows an antibody interaction discovery map of platelet and plasma extracts, using an anti-basic fibroblast growth factor (anti-bFGF) antibody. Specifically, the figure shows that bFGF and fragments thereof are up-regulated in platelets of dormant (non-angiogenic) tumor-bearing mice.

FIG. 23b shows an expression map which allows comparison of the changing expression levels in platelet versus plasma extracts, in addition to differences between expression in bFGF in non-angiogenic and angiogenic tumor bearing mice.

FIG. 24 shows an antibody interaction discovery map of platelet extracts, using an anti-platelet derived growth factor (anti-PDGF) antibody. The figure shows that PDGF and fragments thereof are up-regulated in dormant tumor-bearing mice (30 days after implementation).

FIG. 25 shows an expression map of biomarkers observed after fractionation of platelet extracts on an anion exchange column, followed by profiling of one of those fractions (fraction 1) on a WCX2 ProteinChip array. The figure shows that several markers, including a 20400 Da protein, are up-regulated in platelet extracts taken from tumor-bearing mice (black) compared to platelet extracts from control mice (grey).

FIG. 26 shows an expression map of biomarkers observed after fractionation of platelet extracts on an anion exchange column, followed by profiling of one of those fractions (fraction 1) on a WCX2 ProteinChip array. The figure indicates several markers which were identified to be up-regulated in dormant tumor-bearing mice (black) relative to control mice (grey).

FIGS. 27A-27B: Growth Factor Release from ADP or Thrombin Activated Platelets. The plasma portion of PRP exposed to increasing concentrations of Endostatin was analyzed for VEGF (FIG. 27A) and bFGF (FIG. 27B) using commercially available ELISA. The simple loading of platelets with Endostatin did not release VEGF or bFGF into the supernatant (plasma), and the release of these factors by classical degranulating agents, such as thrombin or ADP was highly selective. Some (but not all) of the VEGF was released by platelet activation with thrombin (but not by ADP). Neither agent was capable of liberating bFGF from platelets.

FIG. 28. Selective VEGF Protein uptake by platelets. VEGF protein was labeled with radioactive iodine and approximately 50 ng of ¹²⁵I-labeled VEGF in 100 μl Matrigel was implanted subcutaneously in the left flanks of C57BLK/6 mice. Three days later the mice were sacrificed and 1 ml of citrated blood was collected by terminal bleed. The radioactivity of each tissue sample was quantified on a gamma counter, the value corrected for differences in tissue weight, and expressed as counts per minute per gm of tissue [cpm/g of tissue]. The experiment was repeated on two separate occasions with 5 mice per experiment, and the graph represents means±standard error.

FIGS. 29A-H: Representative analysis of Platelet Protein Profiles of Tumor-bearing mice. Spectra from healthy mice (“Controls”), mice bearing non-angiogenic dormant tumor xenografts (“non-angiogenic”), and mice bearing angiogenic tumor xenografts (“angiogenic”) are displayed in gel view (FIGS. 29A-29D). Differential expression patterns were detected for several peptide. For example in the basic fraction of the platelet lysate, a band was identified at 8200 Da, and later confirmed to be platelet factor-4 (PF-4) by immunodepletion. Abscises: Relative MW computed from m/z value, Ordinate: Identified peptide confirmed by immunodepletion or immunoprecipitation, Intensity of bands correlates with relative expression profile of the protein (FIGS. 29E-29H).

### DETAILED DESCRIPTION

The present invention relates to methods for the early detection, diagnosis, and treatment of cancer and angiogenic diseases and disorders. In particular, platelets are isolated from a patient at a first time point using standard laboratory procedures for isolating resting platelets (Fujimura H, Thrombos Haemost 2002, 87(4):728-34). The platelets are analyzed for the level of at least one positive or at least one negative angiogenic regulator. At a second, later time point, platelets are isolated from an individual and analyzed for the level of at least one positive or one negative angiogenic regulator. Next, the levels of angiogenic regulators from the platelets of the first sample are compared to the levels of angiogenic regulators from the platelets of the second sample. A change in the level of an angiogenic regulator(s) in the platelets from the second sample, compared to the level of an angiogenic regulator(s) in the first sample is indicative of the presence of an angiogenic disease or disorder, e.g. cancer.

In particular, an increase in the level of at least one positive angiogenic regulator or a decrease in the level of at least one negative angiogenic regulator in the platelets from the second sample, compared to the level of that positive and/or negative angiogenic regulator in the first sample is indicative of the presence of an angiogenic disease or disorder, e.g. cancer.

The positive angiogenic regulators of the present invention include, but are not limited to, VEGF-A (VPC), VEGF-C, bFGF, HGF, angiopoietin-1, PDGF, EGF, IGF-1, IGF BP-3, BDNF, matrix metaloproteinases (MMPs), vitronectin, fibronectin, fibrinogen, heparanase, and sphingosine-1 PO₄.

The negative angiogenic regulators to be analyzed by the present invention include, but are not limited to, PF-4, thrombospondin-1 & 2, NK1, NK2, NK3, fragments of HGF, TGF-beta-1, plasminogen (angiostatin), plasminogen activator inhibitor 1, alpha-2 antiplasmin and fragments thereof, alpha-2 macroglobulin, tissue inhibitors of metaloproteinases (TIMPs), beta-thromboglobulin, endostatin, tumstatin, and soluble VEGFR2.

In addition to known angiogenic regulators, the present invention also encompasses proteins, protein fragments and fusion proteins that have not been traditionally classified as angiogenic regulators, but that are found in platelets. The methods of the present invention provide for the discovery of such proteins.

The cancers to be detected by the methods of the present invention are typically detected at an early stage. For example, the tumor size is in the millimeter range. Such tumors are rarely detected using traditional means of tumor detection, such as, for example, MRI, palpation, mammography, etc. Examples of cancers to be detected include, but are not limited to, gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

Specifically, positive and negative angiogenic regulators that are contained within platelets isolated from the blood of an individual believed to be healthy and disease free, or an individual predisposed to, having, or having been previously treated for cancer may be identified and measured through the methods of the present invention.

Methods for the isolation of platelets are known to those of skill in the art and are described in “Current Protocols in Immunology by F. M. Ausubel, R. Brent, R. E. Kingston, D. D. Moore, J. G. Seidman, K. Struhl and V. B. Chanda (Editors), John Wiley & Sons, 2004.”, incorporated herein by reference. For example, whole blood is collected from a donor into vacutainer containing sodium citrate or other anticoagulant. The whole blood is then centrifuged at low g-force to separate the platelet rich plasma in a first stage from the other components. In a second stage of the procedure, platelet rich plasma is separated into a fresh tube and platelet concentrate obtained by centrifuging platelets at higher speed. The platelet concentrate is then resuspended in a standard lysis buffer and associated proteins are isolated.

The isolation of proteins from cells, including platelets, is known to those of skill in the art and is described in “Current Protocols in Immunology by F. M. Ausubel, R. Brent, R. E. Kingston, D. D. Moore, J. G. Seidman, K. Struhl and V. B. Chanda (Editors), John Wiley & Sons, 2004.”, incorporated herein by reference. In one example, described in WO 02/077176, also incorporated herein by reference, the procedure generally involves the extraction of proteins in one solubilizing step, using a very small volume of a unique buffer. The results of this procedure are intact proteins, substantially free of cross-contamination. The isolated proteins maintain activity, allowing analysis through any number of assays.

The buffers for the protein isolation step can include one or more of buffer components, salt (s), detergents, protease inhibitors, and phosphatase inhibitors. In particular, one effective buffer for extracting proteins to be analyzed by immunohistochemistry includes the buffer Tris-HCl, NaCl, the detergents Nonidet (g) P-40, EDTA, and sodium pyrophosphate, the protease inhibitors aprotinin and leupeptin, and the phosphatase inhibitors sodium deoxycholate, sodium orthovanadate, and 4-2 aminoethylbenzenesulfonylfluororide (AEBSF). Another salt that could be used is LiCl, while glycerol is a suitable emulsifying agent that can be added to the fraction buffer. Additional optional protease inhibitors include soybean trypsin inhibitor and pepstatin. Other suitable phosphatase inhibitors include phenylmethylsulfonyl fluoride, sodium molybdate, sodium fluoride, and betaglycerol phosphate.

For 2-D gel analysis, simple lysis with a 1% SDS solution is effective, while ultimate analysis using the SELDI® process requires Triton-X-100, a detergent (Sigma, St. Louis, Mo.), MEGA109 (ICN, Aurora, Ohio), and octyl B-glucopyranoside (ESA, Chelmsford, Mass.) in a standard PBS base. Another buffer which was used prior to 2-D gel analysis was 7M urea, 2M thiourea, CHAPS, MEGA 10, octyl B-glucopyranoside, Tris, DTT, tributyl phosphine, and Pharmalytes.

Once the proteins have been solubilized, a number of different immunological or biochemical analyses can be used to characterize the isolated proteins. Methods for analysis by ELISA and Western blot are known to those of skill in the art and are further described in “Current Protocols in Molecular Biology by F. M. Ausubel, R. Brent, R. E. Kingston, D. D. Moore, J. G. Seidman, K. Struhl and V. B. Chanda (Editors), John Wiley & Sons, 2004”, incorporated herein by reference. Methods of performing mass spectrometry are known to those of skill in the art and are further described in Methods of Enzymology, Vol. 193:“Mass Spectrometry” (J. A. McCloskey, editor), 1990, Academic Press, New York.

One type of assay that can be performed is a soluble immunoassay, where an antibody specific for a protein of interest is used. The antibody can be labeled with a variety of markers, such as chemiluminescent, fluorescent, or radioactive markers. For best results, a high sensitivity assay can be used, such as a microparticle enzyme immunoassay (MEIA). By applying a calibration curve used to estimate immunodetected molecules in serum, the number of molecules per cell can be estimated. Thus, the presently described methods provide a quantitative immunoassay, which can measure the actual number of the protein molecules of interest in vivo.

A second type of assay that can be used to analyze the extracted proteins is two-dimensional polyacrylamide gel electrophoresis (2D-PAGE). By running both proteins extracted from the first time point and proteins extracted from the second time point, and comparing the blots, differential protein expression can be seen. In particular, by scanning the stained gels into a computer, and using image comparison software, the location of proteins that are present in one cell type and absent (or vice versa) in the other can be determined. Furthermore, these altered proteins can be isolated from the gel where they are present, and mass spectroscopy MS-MS sequencing can be used to identify the protein, if the sequence exists in a database. In this way, the protein differences between the first and the second time points can be more fully understood.

In a preferred embodiment, the analysis is performed using surface enhanced laser desorption ionization spectroscopy technique, or SELDI (Ciphergen Biosystems Inc., Palo Alto, Calif.).

This process can separate proteins that would not be separately focused by 2-D gel analysis, in particular those proteins which are very basic, very small (<7000 Daltons) or are expressed at low or moderate levels in the cells. SELDI also separates proteins more rapidly than gel analysis. SELDI utilizes a “protein chip” that allows for desorption and detection of intact proteins at the femtomole levels from crude samples. Proteins of interest are directly applied to a defined small surface area of the protein chip formatted in 8 to 24 predetermined regions on an aluminum support. These surfaces are coated with defined chemical “bait” matrices comprised of standard chromatographic supports, such as hydrophobic, cationic, or anionic or biochemical bait molecules such as purified protein ligands, receptors, antibodies, or DNA oligonucleotides (see Strauss, Science 282: 1406, 1998). In the case of platelet collected samples, the solubilized proteins are applied to the surface of the SELDI chip. Binding of the proteins to the surface is dependent on the nature of the bait surface and the wash conditions employed. The mixture of bound proteins is then characterized by laser desorption and ionization and subsequent time-offlight (TOF) mass analysis generated from a sensitive molecular weight detector. These data produce a protein fingerprint for the sample, with SELDI having a practical resolution and detection working range of 1000 to 300,000 Daltons, depending on the energy-absorbing molecule utilized and the bait surface/wash conditions employed.

The administration of an effective amount of an anti-cancer therapy having anti-angiogenic activity to a patient is included in the present invention. The anti-cancer therapy may include, for example, administering an angiogenesis inhibitor(s). The angiogenic inhibitor may be administered by traditional methods known to those of skill in the art or by the methods of the present invention, for example, by loading platelets (the patients or a matched donor) with angiogenic inhibitors and administering those loaded platelets to the individual in need. By inhibiting angiogenesis, one can intervene in the disease, ameliorate the symptoms, and in some cases cure the disease. Alternatively, the anti-cancer therapy may involve administering chemotherapy or radiation to the patient. Finally, the anti-cancer therapy may involve surgical resection of a tumor. The treatment may include a combination of the above-mentioned therapies.

The present invention also relates to methods useful in the early detection, diagnosis, and therapeutic treatment of angiogenic diseases or disorders.

There are a variety of diseases or disorders in which angiogenesis is believed to be important, referred to as angiogenic diseases or disorders. As used herein, the term angiogenic disease or disorder or condition is characterized or caused by aberrant or unwanted, e.g. stimulated or suppressed, formation of blood vessels. Aberrant or unwanted angiogenesis may either cause a particular disease directly or exacerbate an existing pathological condition. Examples of angiogenic diseases include ocular disorders, e.g. diabetic retinopathy, macular degeneration, neovascular glaucoma, retinopathy of prematurity, corneal graft rejection, retrolental fibroplasias, rubeosis, retinal neovascularization due to intervention, ocular tumors and trachoma, and other abnormal neovascularization conditions of the eye, where neovascularization may lead to blindness.

Other angiogenic diseases or disorders encompassed in this invention include, but are not limited to, neoplastic diseases, e.g. tumors, including bladder, brain, breast, cervix, colon, rectum, kidney, lung, ovary, pancreas, prostate, stomach and uterus, tumor metastasis, benign tumors, e.g. hemangiomas, acoustic neuromas, neurofibromas, trachomas, and pyrogenic granulomas, hypertrophy, e.g. cardiac hypertophy, inflammatory disorders such as immune and non-immune inflammation, chronic articular rheumatism and psoriasis, disorders associated with inappropriate or inopportune invasion of vessels such as, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth. Also encompassed are lymphoid malignancies, e.g. chronic and acute lymphoid leukemias, and lymphomas. In a preferred embodiment of the present invention, the methods are directed to inhibiting angiogenesis in a mammal with cancer.

The patient to be tested in the present invention in its many embodiments is desirably a human patient, although it is to be understood that the principles of the invention indicate that the invention is effective with respect to all mammals, which are intended to be included in the term “patient”. In this context, a mammal is understood to include any mammalian species.

In an alternative embodiment, the methods of the present invention can be used to stimulate angiogenesis in a patient in need thereof. Platelets have been suggested for drug delivery applications in the treatment of various diseases, as is discussed by U.S. Pat. No. 5,759,542, issued Jun. 2, 1998. This patent discloses the preparation of a complex formed from a fusion drug including an A-chain of a urokinase-type plasminogen activator that is bound to an outer membrane of a platelet. Thus, in accordance with the present invention, platelets may be isolated and associated (“loaded”) with angiogenic stimulating factors. The “loaded” platelets can thus be delivered to sites in need of vascularization.

The methods of the present invention may be used to increase vascularization in patients in need thereof. Thus, the methods of the invention are useful for the treatment of diseases or conditions that benefit from increased blood circulation, for providing a vascularized site for transplantation, for enhancing wound healing, for decreasing scar tissue formation, i.e., following injury or surgery, for conditions that may benefit from directed suppression of the immune response at a particular site, and the like.

Any condition that would benefit from increased blood flow are encompassed such as, for example, gangrene, diabetes, poor circulation, arteriosclerosis, atherosclerosis, coronary artery disease, aortic aneurysm, arterial disease of the lower extremities, cerebrovascular disease, etc. In this manner, the methods of the invention may be used to treat peripheral vascular diseases by pre-loading platelets with angiogenic stimulators and transfusing them into a patient, thus promoting vascularization Likewise, the method is useful to treat a diseased or hypoxic heart, particularly where vessels to the heart are obstructed. Other organs with arterial sclerosis may benefit from the methods Likewise, organs whose function may be enhanced by higher vascularization may be improved by the administration of platelets pre-loaded with angiogenic stimulators. This includes kidneys or other organs which need an improvement in function. In the same manner, other targets for arterial sclerosis include ischemic bowel disease, cerebro-vascular disease, impotence of a vascular basis, and the like. Additionally, formation of new blood vessels in the heart is critically important in protecting the myocardium from the consequences of coronary obstruction. Administration of loaded platelets into a patient having ischemic myocardium can enhance the development of collaterals, accelerate the healing of necrotic tissue and prevent infarct expansion and cardiac dilatation.

Since platelets circulate in newly formed vessels associated with tumors, they could deliver anti-mitotic drugs in a localized fashion, and likely platelets circulating in the neovasculature of tumors can deposit anti-angiogenic drugs so as to block the blood supply to tumors. Platelets loaded with a selected drug, for example, endostatin, displace pro-angiogenic factors such as VEGF or bFGF. In accordance with the present invention, platelets loaded with anti-angiogenic factors can be prepared and transfused into patients for therapeutic applications. The drug-loaded platelets are particularly contemplated for blood-borne drug delivery, such as where the selected drug is targeted to a site of platelet-mediated forming thrombi or vascular injury. The so-loaded platelets have a normal response to at least one agonist, particularly to thrombin. Since tumors demonstrate a physiological upregulation of platelet stimulants such as tissue factor or thrombin, platelets that have been “pre-loaded” with angiogenesis inhibitor(s) would be delivered directly to tumor sites.

Also encompassed in the methods of the present invention is the controlled release of these “pre-loaded” platelets at specific times and/or in specific tissues with agents which are known to release angiogenic regulators from platelets (hereinafter a “release agents”) and in other embodiments with agents which are known to suppress release of angiogenic regulators (hereinafter “suppression agents”).

In one embodiment, the release agent is a proteinase-activated receptor (PAR) agonist. In a preferred embodiment, the PAR agonist is a PAR4 agonist. In another embodiment, the release agent is a PAR1 antagonist. PAR1 and PAR4 agonists and antagonists are known to those of skill in the art and are encompassed in the present invention, see, for example, Ma et al., PNAS, Jan. 4, 2005, vol. 102(1), incorporated herein in its entirety.

Because PAR1 and PAR4 work in a counter-regulatory manner to influence the release of angiogenic regulators from platelets, agonists and antagonists may be administered to patients in need of either suppression or activation of angiogenesis. In this way, the delivery of regulators to sites in need is tailored by the controlled delivery of PAR agonists and antagonists to individuals.

Angiogenesis inhibitors include, but are not limited to, Angiostatin, Bevacizumab (Avastin), Arresten, Canstatin, Caplostatin™, Combretastatin, Endostatin, NM-3, Thrombospondin, Tumstatin, 2-methoxyestradiol, Vitaxin, ZD1839 (Iressa), ZD6474, OSI774 (Tarceva), CI1033, PKI1666, IMC225 (Erbitux), PTK787, SU6668, SU11248, Herceptin, and IFN-α, CELEBREX® (Celecoxib), THALOMID® (Thalidomide), rosiglitazone, bortezomib (Velcade), bisphosphonate zolendronate (Zometa), and IFN-α.

In another embodiment of the present invention, a method for creating a platelet register or profile for an angiogenic disease or disorder is described. This platelet profile is also referred to as a standard. In this embodiment, platelets by isolated from two groups of individuals, one group with a known angiogenic disease or disorder (angiogenic group) and a second group without an angiogenic disease or disorder (control group). The platelets are analyzed for the levels of platelet-associated biomarkers. The average values of the biomarkers are calculated for each group and evaluated to determine the difference between the two groups. A platelet register or profile is then created for the particular angiogenic disease or disorder, where the register lists the biomarkers that are differentially expressed in the angiogenic group as compared to the control group.

The present invention allows for the detection and differentiation of conditions associated with angiogenesis and, in particular, cancer. The invention involves the use of biomolecules found in blood platelets as biomarkers for clinical conditions relating to angiogenesis status and, in particular, cancer status. As used herein, angiogenic status includes, but is not limited to, distinguishing between disease versus non-disease states such as cancer versus normal (i.e., non-cancer) and, in particular, angiogenic cancer versus benign or non-angiogenic cancer.

In fact, it has surprisingly been found that a number of the biomarkers of the present invention can be used distinguish between benign versus malignant tumors, and angiogenic versus non-angiogenic tumors, etc. The selective uptake of angiogenic regulators by platelets, without a corresponding increase of these proteins in plasma, provides a useful measurement to aid in the diagnosis, particularly the early diagnosis, of cancer before a tumor is clinically detected. Moreover, it has been found that the multiplexed measurement of a plurality of biomarkers in platelets, i.e., platelet profiling, provides a very sensitive indication of alterations in angiogenic activity in a patient, and provides disease specific identification. Such platelet properties can be used to detect human cancers of a microscopic size that are undetectable by any presently available diagnostic method. Even a small source of angiogenic proteins, such as a dormant non-angiogenic tumor can modify the protein profile detectably before the tumor itself can be clinically detected. In certain embodiments, the platelet angiogenic profile is more inclusive than a single biomarker because it can detect a wide range of tumor types and tumor sizes. Relative changes in the platelet angiogenic profile permit the tracking of a tumor throughout its development, beginning from an early in situ cancer, i.e., beginning from a point before the tumor is detected clinically, allowing for rapid prognosis, early treatment, and precise monitoring of disease progression or regression (e.g., following treatment with non-toxic drugs such as angiogenesis inhibitors).

Platelets uptake many of the known angiogenic regulatory proteins, e.g., positive regulators such as VEGF-A, VEGF-C, bFGF, HGF, Angiopoietin-1, PDGF, EGF, IGF-1, IGF BP-3, Vitronectin, Fibronectin, Fibrinogen, Heparanase, and Sphingosine-1 P04, and/or negative regulators such as Thrombospondin, the NK1/NK2/NK3 fragments of HGF, TGF-beta-1, Plasminogen (angiostatin), High molecular weight kininogen (domain 5), Fibronection (45 kD fragment), EGF (fragment), Alpha-2 antiplasmin (fragment), Beta-thromboglobulin, Endostatin and BDNF (brain derived neurotrophicfactor), and continue to sequester them for as long as the source (e.g., a tumor) exists. Without limiting the invention to any particular biological mechanism or role for the sequestration of angiogenic regulators, platelets are believed to act as efficient transporters of these proteins to sites of activated endothelium and the profile of biomarkers in the platelets reflects the onset of tumor presence and growth.

In one aspect, the present invention provides a method for qualifying angiogenic status in a subject, the method comprising: (a) measuring at least one platelet-associated biomarker in a biological sample from the subject; and (b) correlating the measurement with angiogenic status.

In one embodiment, the at least one platelet-associated biomarker is measured by capturing the biomarker on an adsorbent of a SELDI probe and detecting the captured biomarkers by laser desorption-ionization mass spectrometry. In certain embodiments, the adsorbent is a cation exchange adsorbent, an anion exchange adsorbent, a metal chelate or a hydrophobic adsorbent. In other embodiments, the adsorbent is a biospecific adsorbent. In another embodiment, the at least one platelet-associated biomarker is measured by immunoassay.

In another embodiment, the correlating is performed by a software classification algorithm. In certain embodiments, the angiogenic status is cancer versus normal (non-cancer). In another embodiment, the angiogenic status is benign tumor versus malignant tumor. In yet another embodiment, the angiogenic status is angiogenic tumor versus non-angiogenic tumor, i.e., dormant, tumor. In yet another embodiment, the angiogenic status is a particular type of cancer, including breast cancer, liver cancer, lung cancer, hemangioblastomas, bladder cancer, prostate cancer, gastric cancer, cancers of the brain, neuroblastomas, colon cancer, carcinomas, sarcomas, leukemia, lymphoma and myolomas.

In yet another embodiment, the method further comprises: (c) managing subject treatment based on the angiogenic status. If the measurement correlates with cancer, then managing subject treatment comprises administering, for example, a chemotherapeutic agent, angiogenic therapy, radiation and/or surgery to the subject.

In a further embodiment, the method further comprises: (d) measuring at least one platelet-associated biomarker after subject management to assess the effectiveness of therapy.

In still another aspect, the present invention provides a kit comprising: (a) a solid support comprising at least one capture reagent attached thereto, wherein the capture reagent binds at least one platelet-associated biomarker; and (b) instructions for using the solid support to detect the at least one biomarker. In another preferred embodiment, the at least one platelet-associated biomarker is selected from the group consisting of the following biomarkers: VEGF, PDGF, bFGF, PF4, CTAPIII, endostatin, tumstatin, tissue inhibitor of metalloprotease, apolipoprotein A1, IL8, TGF, NGAL, MIP, metalloproteases, BDNF, NGF, CTGF, angiogenin, angiopoietins, angiostatin, and thrombospondin and combinations thereof.

In one embodiment, the kit provides instructions for using the solid support to detect a biomarker selected from the following biomarkers: VEGF, PDGF, bFGF, PF4, CTAPIII, endostatin, tumstatin, tissue inhibitor of metalloprotease, apolipoprotein A1, ILS, TGF, NGAL, MIP, metalloproteases, BDNF, NGF, CTGF, angiogenin, angiopoietins, angiostatin, and thrombospondin and combinations thereof.

In another embodiment, the solid support comprising the capture reagent is a SELDI probe. In certain embodiments, the adsorbent is a cation exchange adsorbent, an anion exchange adsorbent, a metal chelate or a hydrophobic adsorbent. In some preferred embodiments, the capture reagent is a cation exchange adsorbent. In other embodiments, the kit additionally comprises (c) an anion exchange chromatography sorbent, such as a quaternary amine sorbent (e.g., BioSepra Q Ceramic HyperD® F sorbent beads). In other embodiments, the kit additionally comprises (c) a container containing at least one of the platelet-associated biomarkers of Table 1 and Table 2.

In a further aspect, the present invention provides a kit comprising: (a) a solid support comprising at least one capture reagent attached thereto, wherein the capture reagent binds at least one platelet-associated biomarker; and (b) a container comprising at least one of the biomarkers.

In one embodiment, the kit provides instructions for using the solid support to detect a biomarker selected from the following biomarkers: VEGF, PDGF, bFGF, PF4, CTAPIII, endostatin, tumstatin, tissue inhibitor of metalloprotease, apolipoprotein A1, IL8, TGF, NGAL, MIP, metalloproteases, BDNF, NGF, CTGF, angiogenin, angiopoietins, angiostatin, and thrombospondin. In another embodiment, the kit provides instructions for using the solid support to detect each of the following biomarkers: VEGF, PDGF, bFGF, PF4, CTAPIII, endostatin, tumstatin, tissue inhibitor of metalloprotease, apolipoprotein A1, IL8, TGF, NGAL, MIP, metalloproteases, BDNF, NGF, CTGF, angiogenin, angiopoietins, angiostatin, and thrombospondin or, alternatively, additionally detecting each of these biomarkers.

In yet a further aspect, the present invention provides a software product, the software product comprising: (a) code that accesses data attributed to a sample, the data comprising measurement of at least one platelet-associated biomarker in the biological sample; and (b) code that executes a classification algorithm that classifies the angiogenic disease status of the sample as a function of the measurement.

In one embodiment, the classification algorithm classifies angiogenic status of the sample as a function of the measurement of a biomarker selected from the group consisting of VEGF, PDGF, bFGF, PF4, CTAPIII, endostatin, tumstatin, tissue inhibitor of metalloprotease, apolipoprotein A1, IL8, TGF, NGAL, MIP, metalloproteases, BDNF, NGF, CTGF, angiogenin, angiopoietins, angiostatin, and thrombospondin. In another embodiment, the classification algorithm classifies angiogenic status of the sample as a function of the measurement of each of the following biomarkers: VEGF, PDGF, bFGF, PF4, CTAPIII, endostatin, tumstatin, tissue inhibitor of metalloprotease, apolipoprotein A 1, IL8, TGF, NGAL, MIP, metalloproteases, BDNF, NGF, CTGF, angiogenin, angiopoietins, angiostatin, and thrombospondin.

In other aspects, the present invention provides purified biomolecules selected from the platelet-associated biomarkers set forth in Table 1 and Table 2 and, additionally, methods comprising detecting a biomarker set forth in Table 1 or Table 2.

A biomarker is an organic biomolecule which is differently present in a sample taken from a subject of one phenotypic status (e.g., having a disease) as compared with another phenotypic status if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

It has been found that platelets are a surprising good source of biomarkers for cancer and for other conditions characterized by differences in angiogenic (including anti-angiogenic) activity. In particular, platelet-derived biomarkers indicate changes in disease status very early, and can distinguish not only cancer from non-cancer, but benign tumors from malignant tumors. As such, the present invention provides a means for early diagnosis of clinical conditions as diverse as cancer, arthritis and pregnancy. Different clinical conditions may be distinguished using the present invention as each clinical condition may result in alteration of a different biomarker or cluster of multiple biomarkers. Thus the biomarker expression pattern for a given clinical condition may be a fingerprint or profile of a disease or metabolic state. Accordingly, the present invention provides kits, methods and devices for detecting and determining expression levels for biomarkers indicative of disease states or alterations in metabolic activity associated with a change in angiogenic activity.

In addition, the present invention provides for the creation of platelet profile standards, or registers. For example, by analyzing platelet samples from individuals with known cancer, one can create a standard profile or register. This register may then be used as a control to compare test samples to. Examples of disease states where platelet profiles will be beneficial include, but are not limited to, breast cancer, liver cancer, lung cancer, hemangioblastomas, bladder cancer, prostate cancer, gastric cancer, cancers of the brain, neuroblastomas, colon cancer, carcinomas, sarcomas, leukemia, lymphoma and myolomas.

The ability of the present invention to detect variations in tumor growth, for example, is illustrated in the Figures and Tables provided herein. The methods used for obtaining the data shown in the Figures and Tables are described in detail in the Examples. Briefly, mice were implanted with either dormant or angiogenic tumors that were allowed to grow for a predetermined period of time. Control animals that were not implanted with a tumor were also surveyed. Platelets were obtained from these mice, homogenated, treated as described in the Examples, and analyzed using SELDI mass spectrometry and other methods practiced by those of ordinary skill in the art. Using this methodology, platelet-derived biomarkers have been identified that can indicate changes in disease status very early, and can distinguish not only cancer from non-cancer, but benign tumors from malignant tumors. For instance, the expression of the biomarker PF4 is enhanced in platelets from mice having tumors. Surprisingly, PF4 expression is highest in those mice having a dormant (non-angiogenic) tumor. The Figures and Table 1 and 2 illustrates a similar result for the biomarker CTAP III, the dimmer of which has a mass of approximately 16.2.

Note that only the molecular weight for a biomarker need be known to make the biomarker suitable for detection, although the shape and intensity of the peaks observed and other parameters may also be used. For example, antibodies to the biomarker may be used or, if the activity of the biomarker is known, an enzyme assay could be used to detect and quantitate the biomarker.

Biomarkers

This invention provides polypeptide-based biomarkers that are differentially present in platelets of subjects having a condition characterized by angiogenic or anti-angiogenic activity, in particular, cancer versus normal (non-cancer) or benign tumor versus malignancy. The biomarkers are characterized by mass-to-change ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The platelet-associated biomarkers of the invention were discovered using SELDI technology employing ProteinChip arrays from Ciphergen Biosystems, Inc. (Fremont, Calif.) (“Ciphergen”). Platelet samples were collected from murine subjects falling into one of three phenotypic statuses: normal, benign tumor, malignant tumor. The platelets were extracted with a urea buffer and then either applied directly to anion exchange, cation exchange or IMAC copper SELDI biochips for analysis, or fractionated on anion exchange beads and then applied to cation exchange SELDI biochips for analysis. Spectra of polypeptides in the samples were generated by time-of-flight mass spectrometry on a Ciphergen PBSII mass spectrometer. The spectra thus contained were analyzed by Ciphergen Express™ Data Manager Software with Biomarker Wizard and Biomarker Pattern Software from Ciphergen Biosystems, Inc. The mass spectra for each group were subjected to scatter plot analysis. A Mann-Whitney test analysis was employed to compare the three different groups, and proteins were selected that differed significantly (p<0.0001) between the two groups. These methods are described in more detail in the Example Section.

The biomarkers of this invention may be characterized by their mass-to-charge ratio as determined by mass spectrometry. The mass-to-charge ratio (“M” value) of each biomarker may also be labeled “Marker.” Thus, for example, M8206 has a measured mass-to-charge ratio of 8206. The mass-to-charge ratios were determined from mass spectra generated on a Ciphergen Biosystems, Inc. PBS II mass spectrometer. This instrument has a mass accuracy of about +/−1000 m/dm, when m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSII, taking the maximum and minimum mass-to-charge-ratio in the cluster, and dividing by two. Accordingly, the masses provided reflect these specifications.

The biomarkers of this invention may further characterized by the shape of their spectral peak in time-of-flight mass spectrometry. Mass spectra showing peaks representing the biomarkers are presented in the Figures.

The biomarkers of this invention may further characterized by their binding properties on chromatographic surfaces. For example, markers found in Fraction III (pH 5 wash) are bound at pH 6 but elute with a wash at pH 5. Most of the biomarkers bind to cation exchange adsorbents (e.g., the Ciphergen® WCX ProteinChip® array) after washing with 50 mM sodium acetate at pH 5, and many bind to IMAC biochips.

The identities of certain biomarkers of this invention have been determined. The method by which this determination was made is described in the Example Section. For biomarkers whose identify has been determined, the presence of the biomarker can be determined by other methods known in the art, including but not limited to photometric and immunological detection.

As biomarkers detectable using the present invention may be characterized by mass-to-charge ratio, binding properties and spectral shape, they may be detected by mass spectrometry without prior knowledge of their specific identity. However, if desired, biomarkers whose identity has not been determined can be identified by, for example, determining the amino acid sequence of the polypeptides. For example, a protein biomarker may be identified by peptide-mapping with a number of enzymes, such as trypsin or V8 protease, and the molecular weights of the digestion fragments used to search databases for sequences that match the molecular weights of the digestion fragments generated by the proteases used in mapping. Alternatively, protein biomarkers may be sequenced using tandem mass spectrometry (MS) technology. In this method, the protein is isolated by, for example, gel electrophoresis. A band containing the biomarker is cut out and the protein subjected to protease digestion. Individual protein fragments are separated by the first mass spectrometer of the tandem MS. The fragment is then subjected to collision-induced cooling. This fragments the peptide producing a polypeptide ladder. The polypeptide ladder may then be analyzed by the second mass spectrometer of the tandem MS. Differences in mass of the members of the polypeptide ladder identifies the amino acids in the sequence. An entire protein may be sequenced this way, or a sequence fragment may be subjected to database mining to find identity candidates.

Use of Modified Forms of a Platelet-Associated Biomarker

It has been found that proteins frequently exist in a sample in a plurality of different forms characterized by a detectably different mass. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention may also be used, themselves, as biomarkers. In certain cases, the modified forms may exhibit better discriminatory power in diagnosis than the specific forms set forth herein.

Modified forms of a biomarker can be initially detected by any methodology that can detect and distinguish the modified forms from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker and modified forms of it. This method will also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified from can be accomplished by any specific detection methodology including affinity capture followed by mass spectrometry, or traditional immunoassay directed specifically the modified form. immunoassay requires biospecific capture reagents, such as antibodies, to capture the analytes. Furthermore, if the assay must be designed to specifically distinguish protein and modified forms of protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

Detection of Platelet-Associated Biomarkers

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant minor method, a grating coupler waveguide method or interferometry).

Prior to detection using the claimed invention, biomarkers may be fractionated to isolate them from other components of blood that may interfere with detection. Fractionation may include platelet isolation from other blood components, sub-cellular fractionation of platelet components, and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209; PCT International Publication No. WO 00/56934; and U.S. Pat. No. 5,242,828.

Detection by Mass Spectrometry

The biomarkers of this invention may be detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

SELDI

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecule^{s} (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and HSO (hydrophobic); SAX-2, Q-10 and LSAX-30 (anion exchange); WCX-2, CM-10 and LWCX-30 (cation exchange); IMAC-3, IMAC-30 and ‘MAC 40 (metal chelate); and PS-10, PS-20 (reactive surface with carboimidizole, expoxide) and PG-20 (protein G coupled through carboimidizole) Hydrophobic ProteinChip arrays have isopropyl or nonylphenoxypoly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); PCT International Publication No. WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Urn et al., “Hydrophobic Surface Chip,” May 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of a-cyano-4-methacryloyloxycinnamic acid and acrylate. In another embodiment, the composition is a co-polymer of a-cyano-4-methacryloyloxycinnamic acid, acrylate and 3-(tri-ethoxy)silyl propyl methacrylate. In another embodiment, the composition is a co-polymer of a-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

Other Mass Spectrometry Methods

In another mass spectrometry method, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

General Protocol for SELDI Detection of Platelet-Associated Biomarkers

As mentioned above, SELDI mass spectrometry is the preferred protocol contemplated by this invention for the detection of the biomarkers. The general protocol for detection of biomarkers using SELDI preferably begins with the sample containing the biomarkers being fractionated, thereby at least partially isolating the biomarker(s) of interest from the other components of the sample. Early fractionation of the sample is preferable as this approach frequently improves sensitivity of the claimed invention. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4, with fractions containing the biomarker being collected.

The sample to be tested (preferably pre-fractionated) is then contacted with an affinity probe comprising an cation exchange adsorbent (preferably a WCX ProteinChip array (Ciphergen Biosystems, Inc.)) or an IMAC adsorbent (preferably an IMAC3 ProteinChip array (Ciphergen Biosystems, Inc.)). The probe is then washed with a buffer that retains the biomarker while washing away unbound molecules. The biomarkers are detected by laser desorption/ionization mass spectrometry.

Alternatively, should antibodies that recognize the biomarker be available, as is the case with PF4 and CTAP III, a biospecific probe may be constructed. Such a probe may be formed by contacting the antibodies to the surface of a functionalized probe such as a pre-activated PSI 0 or PS20 ProteinChip array (Ciphergen Biosystems, Inc.). Once attached to the surface of the probe, the probe may then be used to capture biomarkers from a sample onto the probe surface. The biomarkers then may be detected by, e.g., laser desorption/ionization mass spectrometry.

Detection by Immunoassay

In another embodiment, the biomarkers of this invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

Correlating Changes in Biomarker Expression to Angiogenic Status

Use of the present invention allows the practitioner to diagnose changes in the metabolic state of an individual associated with increased angiogenic activity. This is accomplished by monitoring changes in expression levels of platelet-associated biomarkers resulting from the angiogenic activity associated with the altered metabolic state sought to be detected. Accordingly, preferred biomarkers of the present invention are associated with angiogenesis or angiostasis, although precise identification of suitable biomarkers is not a prerequisite to practicing the claimed invention using those biomarkers. Practice of the claimed invention in the manner described may be performed with a single detectable marker or multiple detectable markers that individually or as a group display altered expression levels in response to modifications of angiogenic activity associated with a physiological modification such as a cancer, infection, pregnancy, tissue injury and the like.

Biomarker expression may be monitored in a variety of ways. For example, a single sample may be analyzed for biomarker expression levels that are subsequently compared to a control threshold determined from sampling a representative control population. Alternatively multiple samples from a single patient taken over a time course may be compared to determine whether biomarker expression levels are increasing or decreasing. This approach is particularly useful when evaluating the prognosis of a patient after treatment for a disease that affects biomarker expression. Still other biomarker evaluations will be readily apparent to one of skill in the art, who may perform the analysis without undue experimentation.

Single Markers

Detection of individual biomarkers is contemplated for the claim invention, provided the biomarker meets the criteria noted above, particularly correlation with the disease or change in metabolic state sought to be detected through use of the invention. Single biomarkers may be used in diagnostic tests to assess angiogenic status in a subject, e.g., to diagnose the presence of cancer or alterations in the course of a disease, such as certain cancers, which affect angiogenic activity in a patient. The phrase “angiogenic status” includes distinguishing, inter alfa, disease v. non-disease states and, in particular, angiogenic cancer v. non-angiogenic dormant cancer. In addition, angiogenic status may include cancers of various types. Based on this status, further procedures may be indicated, including additional diagnostic tests or therapeutic procedures or regimens.

Each of the biomarkers in Table 1A and 1B and Table 2, and others identified by the methods of the present invention are individually useful in aiding in the determination of angiogenic status. Some embodiments of the present invention involve, for example, measuring the expression level of the selected biomarker in a platelet preparation. By comparing the expression level of the biomarker with an earlier-determined expression level in the same individual, one of skill in the art may determine the course of disease, or response of the disease to treatment. Alternatively, the expression level of the detected biomarker may be compared to threshold values for one or more disease states, e.g., as determined by surveying populations of individuals displaying suitable known phenotypes. Exemplary known biomarkers that may be suitable for diagnostic or prognostic purposes by detection individually with the present invention include VEGF, PDGF, bFGF, PF4, CTAPIII, endostatin, tumstatin, tissue inhibitor of metalloprotease, apolipoprotein A1, IL8, TGF, NGAL, MIP, metalloproteases, BDNF, NGF, CTGF, angiogenin, angiopoietins, angiostatin, and thrombospondin.

Use of individual biomarkers as indicators of alterations in angiogenic activity typically involves detecting the biomarker, followed by correlation of the determined biomarker expression level with threshold levels associated with a particular disease or change in metabolic state. For example, capture on a SELDI biochip followed by detection by mass spectrometry and, second, comparing the measurement with a diagnostic amount or cut-off that distinguishes a positive angiogenic status from a negative angiogenic status. The diagnostic amount represents a measured amount of a biomarker above or below which a subject is classified as having a particular angiogenic status. For example, if the biomarker is up-regulated compared to normal during tumor formation, then a measured amount above the diagnostic cutoff provides a diagnosis of cancer. Alternatively, if the biomarker is down-regulated during treatment of an aggressive tumor, then a measured amount below the diagnostic cutoff provides a diagnosis of tumor regression, or passage of the tumor to a dormant state.

The measured level of a biomarker may also be used to facilitate the diagnosis of particular types of cancers or to distinguish between different cancer types. For example, if a biomarker or combination of biomarkers is up-regulated above a particular level in certain types of cancers compared to others, a measured amount of the biomarker above the diagnostic cutoff provides an indication that a particular type of cancer is present. Furthermore, combinations of biomarkers may be used to provide additional diagnostic information, as described below. Some examples of types cancers which may be identified and distinguished from each other using the biomarkers and techniques described herein include breast cancer, liver cancer, lung cancer, hemangioblastomas, neuroblastomas, bladder cancer, prostate cancer, gastric cancer, cancers of the brain, and colon cancer. Carcinomas, sarcomas, leukemia, lymphoma and myolomas may also be distinguished using the biomarkers and methods described herein. Furthermore, different cancer types express different patterns of biomarkers and are distinguished from each other thereby. The patterns characteristic of each cancer type can be determined as described herein by, e.g., analyzing samples from each cancer type with a learning algorithm to generate a classification algorithm that can classify a sample based on cancer type.

As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different angiogenic statuses, as was done here, and drawing the cut-off to suit the diagnostician's desired levels of specificity and sensitivity.

Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. In the context of the present invention, at least two, preferably 3, 4, 5, 6 or 7, more preferably 10, 15 or 20 different biomarker expression levels are determined in the diagnosis of a disease or change in metabolic state. Exemplary biomarkers that may be used in combination include PF4, VEGF, PDGF, bFGF, PDECGF, CTGF, angiogenin, angiopoietins, angiostatin, endostatin, and thrombospondin. A preferred embodiment of the present invention detects a plurality of biomarkers including bFGF and at least one other biomarker selected from the group consisting of VEGF, PDGF, PDECGF, CTGF, angiogenin, angiopoietins, PF4, angiostatin, endostatin, and thrombospondin. An alternative preferred embodiment detects a plurality of biomarkers including PF4 and at least one other biomarker selected from the group consisting of VEGF, PDGF, bFGF, PDECGF, CTGF, angiogenin, angiopoietins, angiostatin, endostatin, and thrombospondin.

Generation of Classification Algorithms for Qualifying Tumor Status

As discussed above, analysis of detected biomarker expression levels may be performed manually or automated using computer software. Single sample analysis may be performed, or multiple sample analysis may be undertaken, with each of the multiple samples being taken from the individual under study at an appropriate time during the course of treatment or evaluation. Accuracy of analysis is particularly important as the determination may be used for both monitoring progress during treatment of a disease or change in metabolic state, and for diagnosing the disease or change in metabolic state. In preferred embodiments of the claimed invention, managing patient treatment is based on categorizing expression levels to accurately reflect the disease or metabolic status of the patient under evaluation.

Many different categorization strategies suitable for use with the present invention are known in the art. A preferable strategy identifies distinct expression levels of a biomarker with distinct stages of disease progression. For example, in tumor growth, the tumor may go through a series of stages from nascent formation to metastasis. Thus a suitable categorization scheme may include “aggressive” characterized by tumor growth and/or metastatic activity; dormant, to identify tumors that are not growing or actively metastasizing; regressive, to identify a tumor that is shrinking, for example after chemotherapy; and no tumor.

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or learning) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 AI (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux“ ” based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for determining angiogenic status. The classification algorithms, in turn, form the base for diagnostic tests by providing diagnostic values (e.g., cut-off points) for biomarkers used singly or in combination.

Managing Patient Care

In providing methods kits and devices for the diagnosis and evaluation of prognosis for disease states, the present invention has utility in providing tools for management of patient care. In particular, the present invention finds use in diagnosing and evaluating the treatment of a variety of diseases that lead to a change in angiogenic activity in the patient. Such conditions may include, for example, cancer, pregnancy, infection (e.g., hepatitis), injury, and arthritic conditions. In certain embodiments of the present invention, methods of qualifying angiogenic status, the methods further comprise managing subject treatment based on the status. Such management includes the actions of the physician or clinician subsequent to determining disease status. For example, if a physician makes a diagnosis of aggressive cancer, then a certain regime of treatment, such as chemotherapy or surgery might follow. Alternatively, a diagnosis of no tumor or dormant tumor might be followed with further testing to determine a specific disease afflicting the patient.

A particularly useful aspect of the present invention is that it provides for early detection of potentially life-threatening conditions, as noted above. Early diagnosis enhances the prognosis for recovery by allowing early treatment of the condition. By way of example, early detection of cancer allows for earlier and less debilitating chemotherapy or surgical removal of any tumor prior to metastasis. Early detection of arthritis allows for drug intervention to control inflammation before debilitating joint injury occurs, slowing the symptoms of the disease.

After diagnosis, detecting biomarkers using the present invention allows evaluation of the effectiveness of the treatment regime being employed. For example, in cancers, detecting a decrease in expression of the CTAP III biomarker after treatment of a dormant tumor correlates with the tumor altering phenotype to an aggressive tumor. Conversely, detecting a subsequent increase in CTAP III correlates with a change in the tumor phenotype from aggressive to dormant or absent.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of a biomarker indicative of a disease or metabolic state is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

Diagnostic Systems

The present invention also contemplates diagnostic systems for detecting biomarkers whose expression is altered in response to changes in angiogenic activity in a patient. The diagnostic systems of the invention are preferably operated in a single step, but are not limited to such. For example some embodiments comprise a plurality of adsorbent surfaces binding a plurality of platelet-associated biomarkers. Preferably, the adsorbents are biospecific adsorbents that specifically adsorb the biomarkers of interest. The diagnostic systems of the invention also have a means for detecting the biomarkers of interest, which maybe a mass spectrometer.

By way of example, a preferred embodiment of the present invention accepts a plasma homogenate on a sintered frit. The frit is in fluid communication with a bibulous material capable of supporting capillary flow of a liquid. Within the bibulous material are reagents, including a fluidly mobile biospecific adsorbent that specifically recognizes the biomarker to be detected. Preferably, the fluidly mobile biospecific adsorbent includes a detectable label, more preferably, a visible label. Further downstream in the bibulous material is a fixed biospecific adsorbent recognizing the biomarker to be detected.

Using a simple device, such as that described above, a plasma homogenate introduced to the sintered frit is filtered free of cellular debris. The remaining liquid progresses to the bibulous material, which wicks the liquid into and ultimately along its length. In traversing the bibulous material, the fluidly mobile biospecific adsorbent is solublized and binds to the biomarker to be detected forming a complex. As the liquid progresses further through the bibulous material, the complex encounters and binds to the fixed biospecific adsorbent. As the complex binds to the fixed biospecific adsorbent, it becomes concentrated at the point where the fixed biospecific adsorbent is attached to the bibulous material, where it may be detected. The device may optionally be washed with a wash buffer after complex binding to remove potentially interfering material present in the original homogenate.

One of skill in the art will readily recognize that there are several variant device formats that perform in substantially the same manner as the preferred device described above. For example, the device could essentially be performed in an ELISA-type manner using biospecific reagents coupled to the floor of microtitre plate wells. In this format, the homogenate is added to a well. Excess homogenate is then removed and the well washed with a wash buffer. Finally, the labeled mobile antibody is added and the resulting complex detected.

One of skill in the art will readily recognize the format of the device described above as being well known, with many variants falling within the scope of the present invention. For example, similar devices are described in U.S. Pat. Nos. 5,409,664, 6,146,589, 4,960,691, 5,260,193, 5,202,268 and 5,766,961.

Use of biomarkers for cancer in screening assays and methods of treating cancer

The methods of the present invention have other applications as well. For example, the biomarkers can be used to screen for compounds that modulate the expression of the biomarkers in vitro or in vivo, which compounds in turn may be useful in treating or preventing cancer in patients or in treating or preventing the transformation of a tumor from a dormant tumor to an aggressive tumor. In another example, the biomarkers can be used to monitor the response to treatments for cancer. In yet another example, the biomarkers can be used in heredity studies to determine if the subject is at risk for developing cancer.

Thus, for example, the kits of this invention could include a solid substrate having a hydrophobic function, such as a protein biochip (e.g., a Ciphergen HSO ProteinChip array, e.g., ProteinChip array) and a sodium acetate buffer for washing the substrate, as well as instructions providing a protocol to measure the platelet-associated biomarkers of this invention on the chip and to use these measurements to diagnose, for example, cancer.

Compounds suitable for therapeutic testing may be screened initially by identifying compounds which interact with one or more biomarkers listed in Table 1A and 1B and Table 2. By way of example, screening might include recombinantly expressing a biomarker listed in Table 1A and 1B and Table 2, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1A and 1B and Table 2, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1A and 1B and Table 2 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers in the tables may also be measured. For example, the self-assembly of a multi-protein complex which includes one of the biomarkers in the tables may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers in the tables may be administered to patients who are suffering from or are at risk of developing cancer. For example, the administration of a test compound which increases the activity of a particular biomarker may decrease the risk of cancer in a patient if the activity of the particular biomarker in vivo prevents the accumulation of proteins for cancer. Conversely, the administration of a test compound which decreases the activity of a particular biomarker may decrease the risk of cancer in a patient if the increased activity of the biomarker is responsible, at least in part, for the onset of cancer.

In an additional aspect, the invention provides a method for identifying compounds useful for the treatment of disorders such as cancer which are associated with increased levels of modified forms of the platelet-associated biomarkers of the tables. For example, in one embodiment, cell extracts or expression libraries may be screened for compounds which catalyze the cleavage of the full-length biomarkers to form truncated forms. In one embodiment of such a screening assay, cleavage of the biomarkers may be detected by attaching a fluorophore to the biomarker which remains quenched when biomarker is uncleaved but which fluoresces when the biomarker is cleaved. Alternatively, a version of full-length biomarker modified so as to render the amide bond between certain amino acids uncleavable may be used to selectively bind or “trap” the cellular protesase which cleaves the full-length biomarker at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the progression or likelihood of a disease, e.g., cancer, which is associated with the increased levels of a truncated biomarker. For example, after one or more proteins have been identified which cleave a full-length biomarkers of the tables, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of the platelet-associated biomarker.

At the clinical level, screening a test compound includes obtaining samples from test subjects before and after the subjects have been exposed to a test compound. The levels in the samples of one or more of the platelet-associated biomarkers listed in the tables may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in the tables may be measured directly by Western blot using radio- or fluorescently-labeled antibodies which specifically bind to the biomarkers.

### EXAMPLES

Circulating platelets contain a variety of regulators that can modify the angiogenic process. The platelets' ability to adhere to abnormal surfaces and release their contents within the local environment makes them a highly desirable modality for local angiogenic factor delivery. In physiological situations of angiogenesis, this strictly local release of growth factors represents a highly flexible, safe and effective system for wound healing or reproduction; but in pathological situations, such as cancer, chronic inflammatory disorders or vascular anomalies, it represents a critical paracrine amplification loop for growth.

Platelets have numerous mechanisms for this controlled, highly gradated and locally responsive action:
i) Platelet microparticles (PMPs) are shed throughout tumor progression: It is well known that tumor vasculature, mainly because of its fenestration, and highly irregular endothelial cell surface, activates platelets; and PMPs containing VEGF, bFGF and other growth factors are released into the systemic circulation without any obvious paraneoplastic thrombotic events.
ii) α-granules store growth factors and inhibitors which can be released in response to local stimuli: the contents of platelet granules depend on the local milieu of the host and as such reflect a “tumor register”.
iii) More than one process participates in tumor progression and dissemination: PMPs maintain low-level continuous delivery of growth factors, and α-granules provide fast, and localized amplification of pro-angiogenic signals.

We refer to the platelet profile of angiogenic growth factors and inhibitors as “platelet register”. This platelet register can be used for diagnostic, as well as therapeutic purposes.

The goal of our experiments were to:
1. identify angiogenesis or tumor-related growth factors or inhibitors transferred by platelets, i.e. tumor profile.
2. identify the storage system in the platelets, i.e. granules, dense-granules or membrane particles.
3. investigate the mechanism of transport of these compounds (i.e. define the stimuli for granules' release).
4. define the clinical situations in which PMP are the main mechanism of platelet activity and circumstances where platelet aggregation and de-granulation are necessary for local factor release.

Study Phases:

Phase 1: Platelet samples from non-tumor bearing SCID and C57 Bl mice are isolated and profiled.

Phase 2: Platelets from non-tumor bearing SCID mice are separated into membrane and cytoplasmic fractions and the factor content compared to whole platelet extracts to determine the transport system for the specific proteins.

Phase 3: Protein profiles of platelets of tumor-bearing SCID mice are compared to the protein profiles of pure tumor cell extracts to correlate the relevance of the transported growth factors and inhibitors.

Phase 4: Platelet samples from SCID mice bearing dormant (non-angiogenic) tumors and SCID mice bearing fast growing (angiogenic) tumors are compared with age-matched non-tumor bearing mice of the same background.

Phase 5: Plasma from SCID mice bearing dormant (non-angiogenic) tumors and SCID mice bearing fast growing (angiogenic) tumors are compared with age-matched non-tumor bearing mice of the same background (plasma is used as surrogate for the factors released continuously into the circulation, i.e. without any aggregation and de-granulation of platelets).

Phase 6: Sera from SCID mice bearing dormant (non-angiogenic) tumors and SCID mice bearing fast growing (angiogenic) tumors are compared with age-matched non-tumor bearing mice of the same background (sera is used as surrogate for the factors released upon aggregation and de-granulation of activated platelets).

Previous reports have suggested that platelets contain and transport proteins and that this protein is taken into platelets down a concentration gradient from the plasma. However, our results show that a relatively small source of VEGF such as a Matrigel pellet or microscopic (0.5-1 mm³) tumor can contribute their VEGF directly to platelets without ever raising plasma levels of VEGF. Most importantly, the presence of a microscopic, clinically undetectable tumor is enough to induce platelets of SCID mice bearing human lipo sarcoma to pick up specific angiogenic regulators and change the “resting” protein profile to a “tumor-reflecting” profile.

We further confirm that i) the proteins sequestered in platelets in the presence of tumor growth are predominantly angiogenic regulators such as VEGF, bFGF, PDGF, PF4, Endostatin, angiostatin, and tumstatin, rather than the most abundant plasma proteins such as albumin and ii) the levels of angiogenic regulators in platelets vary depending on presence of tumors or other sources of angiogenic factors.

We hypothesized that the excess of angiogenic growth factors resulting from oncogenic transformation is reflected in platelets early in tumorigenesis, when plasma and serum levels of tumor markers are negligible. In the study presented herein, we confirm the ability of platelets to accumulate selected proteins both in vivo and in vitro and show a selective replacement of one angiogenic regulator with another. Because of the multiplicity of regulators such as growth factors, inhibitors, co-factors and cytokines involved in tumor progression, we have used a high through-put SELDI-ToF MS (Surface enhanced laser desorption/ionization-time of flight mass spectrometry) to analyze protein profiles of purified platelets and plasma. The technology allows for mass spectroscopy analysis of large number of clinical samples at one time and provides an efficient, highly reproducible way for comparisons of entire platelet proteomes.

Comparing platelet profiles of age-matched healthy SCID mice littermates bearing human tumor xenografts of liposarcoma with those of sham injected non-tumor bearing animals. In agreement with the numerous reports of proteins contained in platelets (10-12) we found that at least 21 positive regulators of endothelial proliferation and migration as well as at least 15 negative regulators of endothelial proliferation and migration coexist in platelets. The analysis of the corresponding plasma samples from mice bearing human tumor xenografts demonstrated no significant differences in these regulatory proteins.

The novel finding of this study was the customization of platelet profiles in presence of tumors. We present data that platelets have ability to detect sub-clinical tumor growth, respond to tumor presence early in the process of tumorigenesis by selective uptake of angiogenic regulators, sequester and protect these proteins from degradation while in circulation and possibly facilitate transport of those proteins to tumor sites. This localization of platelet action may act to enhance tumor angiogenesis while evading much of the host surveillance controls, or, such as in the case of tumor dormancy, maintain the necessary level of angiogenesis inhibitors to stall tumor growth.

Platelets represent a very sophisticated system for the trafficking of angiogenesis regulators and a clinically applicable analysis of their protein profiles affords us the ability to diagnose cancer earlier than presently possible.

Methods

In Vitro Endostatin Uptake by Freshly Isolated Platelets.

Platelet rich plasma (PRP) was isolated from the blood of healthy human volunteers by centrifugation of citrated whole blood at 200 g for 20 minutes. The platelet rich plasma was transferred to a fresh polyethylene tube and incubated on a gentle rocker at room temperature for one hour with increasing concentrations of human recombinant endostatin (EntreMed. Inc., Rockville, Md.). Following incubation, the PRP was centrifuged at 800 g to pellet the platelets and the supernatant (platelet poor plasma [PPP]) was saved for analysis by ELIZA at a later stage. Platelets were then gently re-suspended in Tyrodes buffer containing 1 U/ml PGE2 and pelleted again. The wash was repeated twice in this manner before removing the membrane fraction of platelets by centrifugation with Triton X, and lysing the pellet for standard SDS-PAGE analysis. Platelets were lysed using 50 mM Tris HCL, 100-120 mM NaCl, 5 mM EDTA, 1% Igepal and Protease Inhibitor Tablet (complete TM mixture, Boehringer Manheim, Indianopolis, Ind.). Protein concentrations were equalized using standard Bradford method (Bio-Rad Laboratories Inc., Hercules, Calif.), and an equivalent amount of either endostatin protein standard or platelet protein lysate was mixed with sample buffer (Invitrogen, Carlsbad, Calif.) and loaded onto a 12% SDS-polyacrylamide gel (Invitrogen, Carlsbad, Calif.). Following transfer to a PVDF membrane (Millipore, Billerica, Mass.), the mixture was blocked with 7% milk and incubated with the following antibodies: anti-human endostatin (courtesy of Kashi Javaherian, Childrens Hospital, Boston), anti-human VEGF (1:1000, Santa Cruz Biotechnology Inc., Santa Cruz, Calif.), or anti-human bFGF (1:1000, Upstate USA Inc., Charlottesville, Va.). Positive signals were then detected using a Super Signal West Pico Chemiluminescence Kit (Pierce Biotechnology inc., Rockford, Ill.) and autoradiography.

In Vivo ¹²⁵I-Labelled VEGF Uptake by Platelets.

Iodination of VEGF protein was performed according to previously established methods. Briefly, Iodo Beads® (Pierce Biotechnology Inc., Rockford, Ill.) pre-equilibrated with 10 μl sodium phosphate buffer (SPB, 0.2M NaHPO4, pH 7.2) were incubated with 10 μg of carrier-free rmVEGF (R&D Systems Inc., Minneapolis, Minn.) and 1 mCu of 125Iodine. The sample was further diluted with 150 μl of sodium phosphate buffer and passed through a 15 ml, pre-equilibrated NAD™ 5 column (Amersham Biosciences, Piscataway, N.J.) containing 0.2% gelatin in PBS. Fifteen fractions of 250 μl were then collected. Radioactivity in each fraction was quantified on a Gamma 5000 Beckman Iodine 125 (Beckman Instruments, Fullerton, Calif.) and the two fractions containing the greatest quantity of ¹²⁵I-labeled VEGF (500 μl in total) were combined for use in the Matrigel assay on the day of the experiment. Briefly, the left flanks of C57Bl/6 mice were shaved one day prior to Matrigel pellet implantation to avoid a minor cutaneous inflammatory reaction. On the day of the experiment, 500 μl of 125 I-VEGF in buffer was mixed with 500 μl growth factor free Matrigel (B & D Biosciences, Bedford, Mass.) and 100 μl of this mixture was injected subcutaneously into the left flank of each mouse. Three days later the mice were anesthetized using inhalational anesthesia (2% isofluorane in 1 L of oxygen), and 1 ml of whole blood was drawn into a citrated syringe (1% sodium citrate final concentration, 1/10 v/v) by direct cardiac puncture without opening the chest cavity.

The platelets were isolated in two centrifugation steps: the first at 200 g to isolate platelet rich plasma (PRP), followed by centrifugation at 800 g to yield a platelet pellet and a platelet-poor plasma fraction (PPP). The radioactivity of each platelet sample was quantified on a gamma counter. The value was corrected for differences in tissue weight and expressed as counts per minute per gram of tissue [cpm/g of tissue].

Tumor Cell and Xenograft Models.

Non-angiogenic and angiogenic tumor xenografts of human liposarcoma (SW872) sub-clones, which form either non-angiogenic, microscopic, dormant tumors, or angiogenic rapidly growing tumors in immuno-deficient mice, were used as an in vivo experimental system 7. Other human tumors including breast cancer, colon cancer, glioblastoma and osteosarcoma have also been subcloned into non-angiogenic and angiogenic tumor cell populations. All the human non-angiogenic tumor subclones undergo a switch to the angiogenic phenotype at a predictable time in vivo, i.e., 133 days median±2 weeks for liposarcoma, 80 days for breast cancer. However, only in liposarcoma does the angiogenic switch occur in 100% of non-angiogenic tumors and the tumor is used here to demonstrate the differences. The liposarcoma (SW872) tumor cell line sub-clones were each derived from a single cell: clone 4 is non-angiogenic and remains dormant and microscopic for a median of ˜133 days before becoming angiogenic and undergoing rapid tumor expansion. Clone 9 is angiogenic at the time of implantation and expands rapidly. The tumor cell proliferation rates are equivalent for clone 4 and clone 9, in vivo and in vitro. However, the tumor cell apoptotic rate in vivo was high in the non-angiogenic clone 4 and low in the angiogenic clone 9 (Folkman/Almog submitted for publication).

All cell lines were cultured in DMEM containing 5% heat inactivated fetal bovine serum (HyClone, Logan, Utah), 1% antibiotics (penicillin, streptomycin) and 0.29 mg/ml L-glutamine in a humidified 5% CO2 incubator at 37° C. For injections into mice, 80-90% confluent tumor cells were rinsed in phosphate-buffered saline (PBS) (Sigma, St. Louis, Mo.), briefly trypsinized and suspended in serum-free DMEM. The cells were washed in twice in DMEM, and their final concentration was adjusted to 5×106 viable cells/200 μl.

Six-week old male SCID mice from the Massachusetts General Hospital (MGH), Boston, Mass. were injected subcutaneously in the flanks with 5×106 cells (in 0.2 ml) from a single clone. All experiments were conducted in compliance with Boston Children's Hospital guidelines using protocols approved by the Institutional Animal Care and Use Committee.

Platelet, Plasma and Tumor Processing and Protein Profiling.

Blood was collected from anesthetized mice by direct cardiac puncture into citrated polyethylene tubes (1% sodium citrate final concentration, 1/10 v/v) and centrifuged immediately at 200 g. The upper phase, PRP, was then transferred into a fresh tube, and platelets were separated by further centrifugation at 800 g. The isolated platelet pellet (P) and platelet poor plasma (PPP) supernatant were analyzed separately using SELDI-TOF technology (Ciphergen®, Freemont, Calif.).

Platelet pellets from each mouse were processed in 9M urea (U9), 2% CHAPS (3-[(3-cholamidopropyl)dimethylammonio]-1-propansulfonate), 50 mM TrisHCl, pH 9; centrifuged at 10,000 g at 4° C. for 1 min, and platelet extracts were fractionated as described below. From each mouse, 200 of PPP was denatured with 400 of U9 buffer (9M urea, 2% CHAPS, 50 mM TrisHCl, pH 9), and the pure plasma extract was fractionated by anion-exchange chromatography modified after the Expression Difference Mapping (EDM) Serum Fractionation protocol (Ciphergen®, Fremont, Calif.). The fractionation was performed in a 96-well format filter plate on a Beckman Biomek® 2000 Laboratory Work Station equipped with a DPC® Micromix 5 shaker. An aliquot of 200 of the platelet and tumor extract, and 60 μl of denatured plasma diluted with 100 μl of 50 mM Tris-HCl pH9 was transferred to a filter bottom 96-well microplate pre-filled with BioSepra Q Ceramic HyperD® F sorbent beads rehydrated with 50 mM TrisHCl, pH 9, and pre-equilibrated with 50 mM Tris-HCl, pH 9. All liquids were removed from the filtration plate using a multiscreen vacuum manifold (Millipore, Bedford, Mass.). After incubating for 30 min at 4° C., the flow-through was collected as Fraction I. The filtration plate was incubated with 2×100 μl of the following buffers to yield the following fractions: 1M urea, 0.1% CHAPS, 50 mM NaCl, 2.5% acetonitrile, 50 mM Tris-HCl (pH 7.5, Fraction II); 1M urea, 0.1% CHAPS, 50 mM NaCl, 2.5% acetonitrile 50 mM NaAcetate (pH 5.0, Fraction III); 1M urea, 0.1% CHAPS, 50 mM NaCl, 2.5% acetonitrile 50 mM NaAcetate (pH 4.0, Fraction IV); 1M urea, 0.1% CHAPS, 500 mM NaCl, 2.5% acetonitrile 50 mM NaCitrate (pH 3.0, Fraction V) and 33.3% isopropanol/16.7% acetonitrile/8% formic acid (organic phase, Fraction VI).

Expression difference mapping (EDM) on ProteinChip® arrays was carried out using weak cationic exchange chromatography protein arrays (WCX2 ProteinChip™ arrays; Ciphergen®, Fremont, Calif.) by loading sample fractions onto a 96-well bioprocessor, and equilibrating with 50 mM sodium acetate 0.1% octyl glucoside (Sigma, St. Louis, Mo.), pH 5.0. A further dilution of 40 μl anion exchange chromatography fraction into 1000 of the same buffer on each array spot was incubated for an hour. Array spots were washed for 3 minutes with 100 μl 50 mM sodium acetate 0.1% octyl glucoside pH 5. After rinsing with water, 2×1 μl of sinapinic acid matrix solution was added to each array spot.

For protein profiling, all fractions were diluted 1:2.5 in their respective buffers used to pre-equilibrate ProteinChip® arrays. This step was followed by followed by readings using the Protein Biology System II SELDI-ToF mass spectrometer (Ciphergen®, Fremont, Calif.). The reader was externally calibrated daily using protein standards (Ciphergen®, Fremont, Calif.) as calibrants. Spectra were processed with the ProteinChip Software Biomarker Edition®, Version 3.2.0 (Ciphergen, Fremont, Calif.). After baseline subtraction, spectra were normalized by means of a total ion current method. Peak detection was performed by using Biomarker Wizard software (Ciphergen, Fremont, Calif.) employing a signal-to-noise ratio of 3.

Candidate protein biomarkers were further purified by affinity chromatography on IgG spin columns and by reverse phase chromatography. The purity of each step was monitored by employing Normal Phase (NP) ProteinChip® arrays. The main fractions were reduced by 5 mM DTT pH 9 and alkylated with 50 mM iodoacetamide in the dark for 2 hours. The final separation was on a 16% Tricine SDS-PAGE gel. The gel was stained by Colloidal Blue Staining Kit (Invitrogen, Carlsbad, Calif.). Selected protein bands were excised, washed with 200 μl of 50% methanol/10% acetic acid for 30 min, dehydrated with 100 μl of acetonitrile (ACN) for 15 minutes, and extracted with 70 μl of 50% formic acid, 25% ACN, 15% isopropanol, and 10% water for 2 hours at room temperature with vigorous shaking. The candidate biomarkers in extracts were again verified by analysis of 2 μl on a Normal Phase ProteinChip array. The remaining extract was digested with 20 μl of 10 ng/μl of modified trypsin (Roche Applied Science, Indianapolis, Ind.) in 50 mM ammonium bicarbonate (pH 8) for 3 hours at 37° C. Single MS and MS/MS spectra were acquired on a QSTAR mass spectrometer equipped with a Ciphergen PCI-1000 ProteinChip Interface. A 1 μl aliquot of each protease digest was analysed on an NP20 ProteinChip Array in the presence of CHCA. Spectra were collected from 0.9 to 3 kDa in single MS mode. After reviewing the spectra, specific ions were selected and introduced into the collision cell for CID fragmentation. The CID spectral data was submitted to the database-mining tool Mascot (Matrix Sciences) for identification.

Immunofluorescence Microscopy.

Anti-VEGF mouse monoclonal antibody was obtained from Becton Dickinson Biosciences and used at 5 μg/ml. Rabbit anti-β1 tubulin antiserum (a kind gift from Nicholas Cowan, Brigham and Women's Hospital, Boston) and was used at 1:1000 dilution. Alexa 488 anti-rabbit and Alexa 568 anti-mouse secondary antibodies with minimal cross-species reactivity were purchased from Jackson Immuno Research Laboratories (West Grove, Pa.). Cells were analyzed on a Zeiss Axivert 200 microscope equipped with a 100× objective (NA 1.4), and a 100-W mercury lamp. Images were acquired with an Orca II cooled charged coupled device (CCD) camera (Hamamatsu). Electron shutters and image acquisition were under the control of Metamorph software.

Resting platelets were fixed for 20 minutes in suspension by the addition of 3.7% formaldehyde. The platelets were attached to polylysine-coated coverslips placed in wells of a 12-well microtiter plate and centrifuged at 250 g for 5 minutes. For agonist-induced activation, platelets were sedimented onto coverslips in an identical fashion and 1 U/ml thrombin was added for 5 min. Activated platelets were fixed for 20 minutes in 3.7% formaldehyde. Samples were permeabilized in Hanks' solution containing 0.5% Triton X-100 and washed with PBS. Specimens were blocked overnight in PBS+1% BSA, incubated in primary antibody for 2-3 hours at room temperature, washed, treated with appropriate secondary antibody for 1 hour, and again washed extensively in 1% PBS. Primary antibodies were used at 1 mg/ml in PBS+1% BSA and secondary antibodies at a 1:500 dilution in the same buffer. Controls were processed identically except for omission of the primary antibody.

Results:

Active and Selective Uptake of Angiogenesis Regulatory Proteins by Platelets In Vitro.

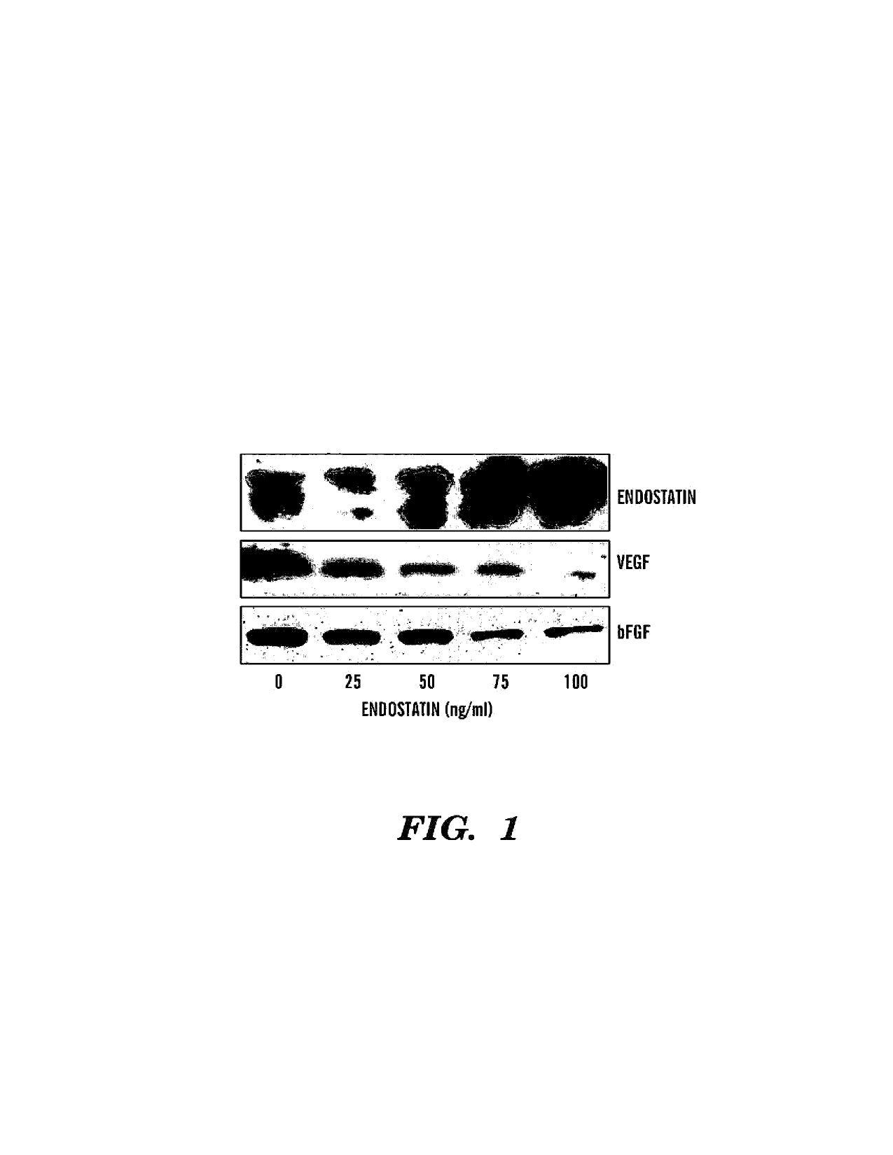

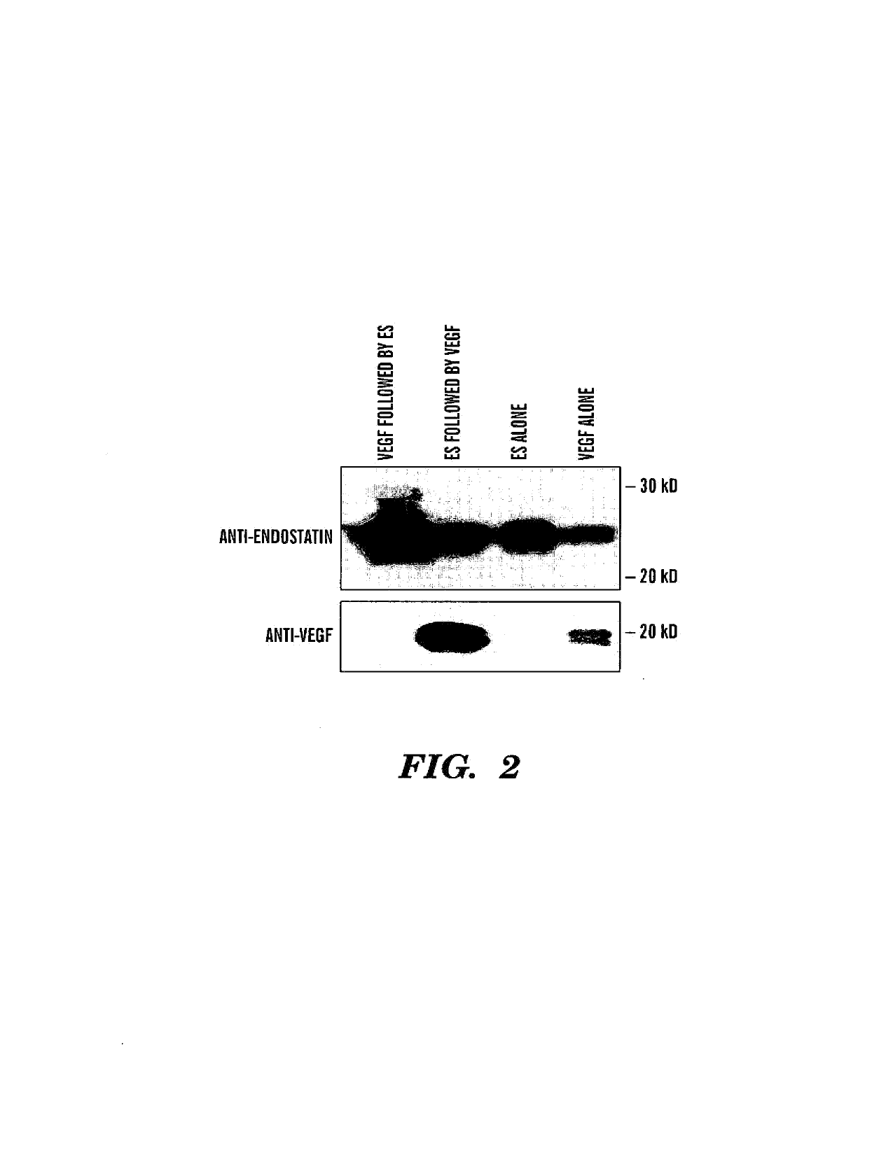

Platelets incubated with increasing concentrations of human recombinant endostatin take up the protein in a dose-dependent manner (FIG. 1, upper blot). A semi-quantitative SDS-PAGE analysis reveals that as the endostatin load into the platelets increases, it causes cytoplasmic re-distribution of other native platelet proteins, such as VEGF and bFGF (FIG. 1, lower two blots). Because the platelet surface expresses a high level of nonspecific protein binding sites, the platelet membrane fraction was removed by centrifugation with Triton-X100 before protein lysis. To explore whether the process of protein uptake by platelets is a random phenomenon or an inherent mechanism of sequestration, we subsequently challenged the platelets by the addition of the indicated proteins in a predetermined sequential fashion. We found that platelets preloaded with endostatin exhibited limited VEGF uptake when added to the assay, resulting in some, but not complete, decrease in the cytoplasmic levels of endostatin. Conversely, endostatin was able to cause much more complete re-distribution of the preloaded VEGF (FIG. 2).

Active and Selective Uptake of Angiogenesis Regulatory Proteins by Platelets In Vivo.

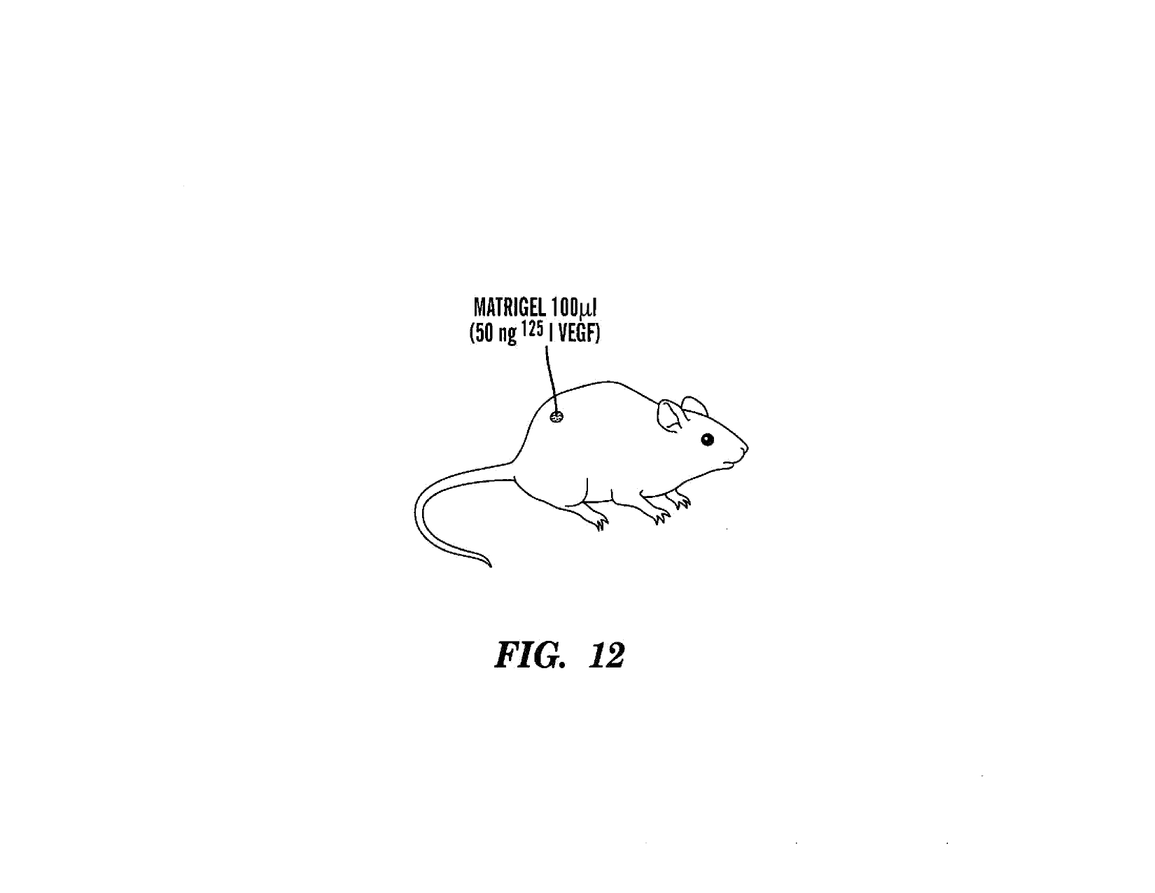

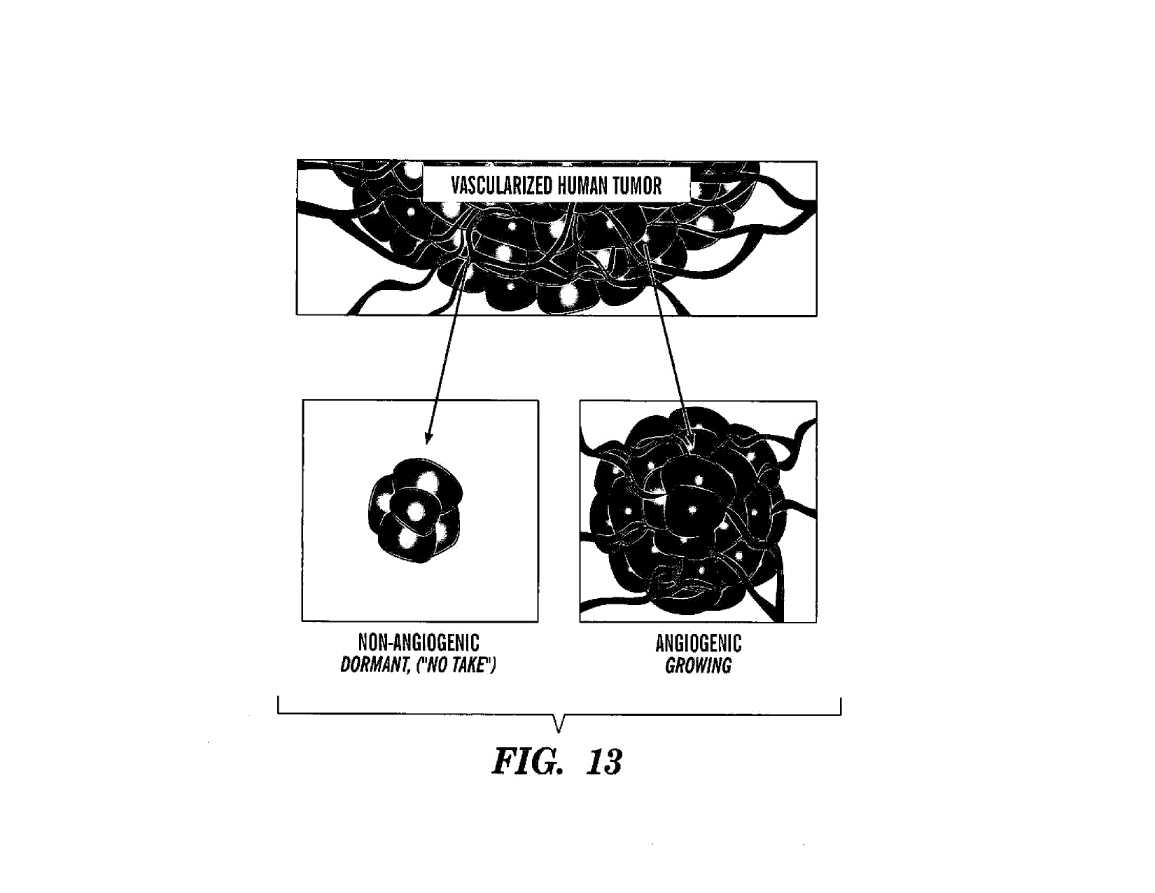

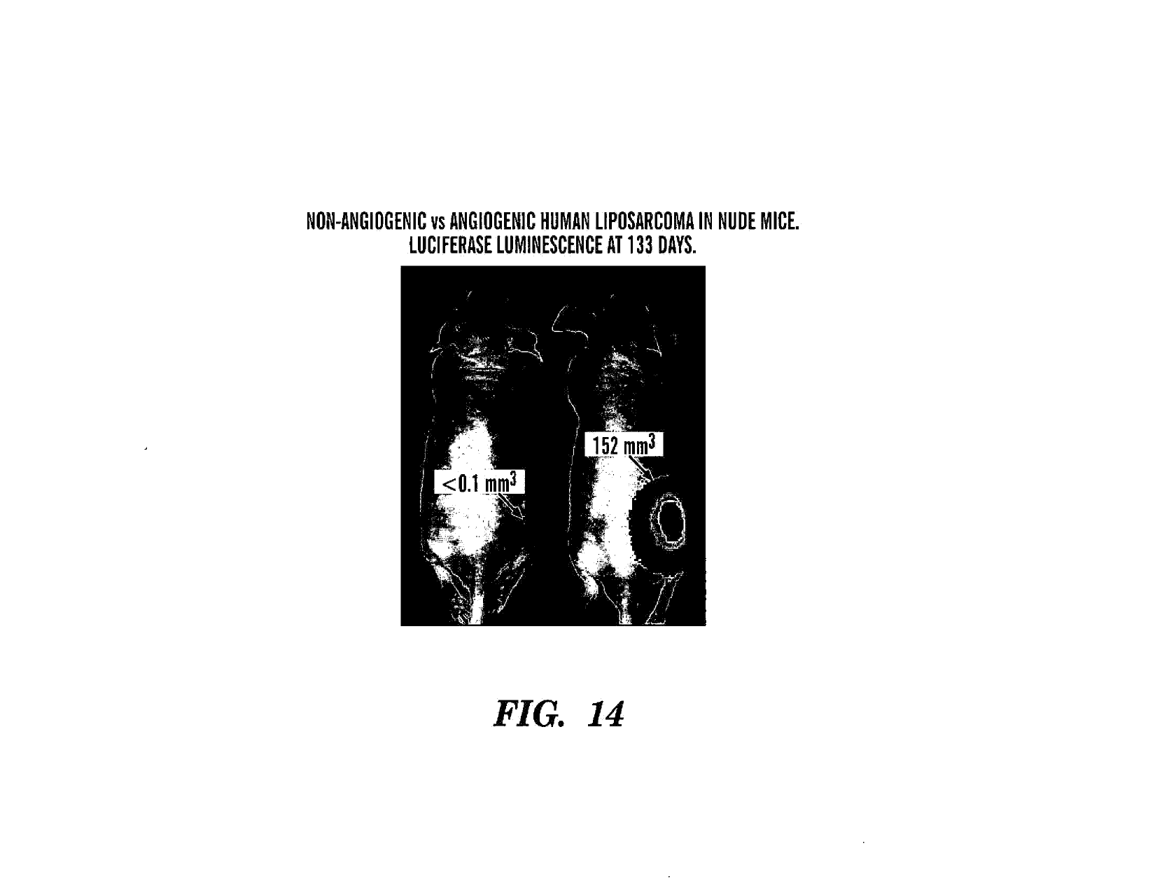

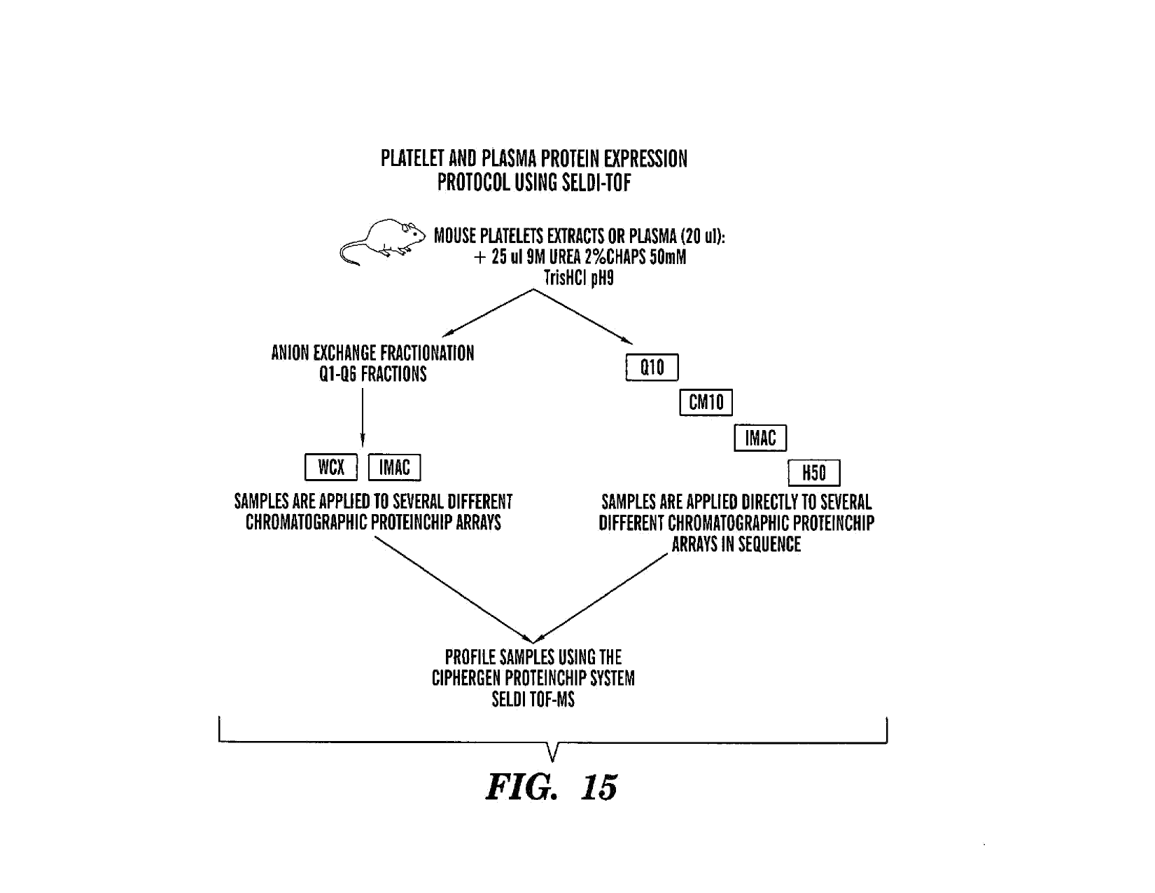

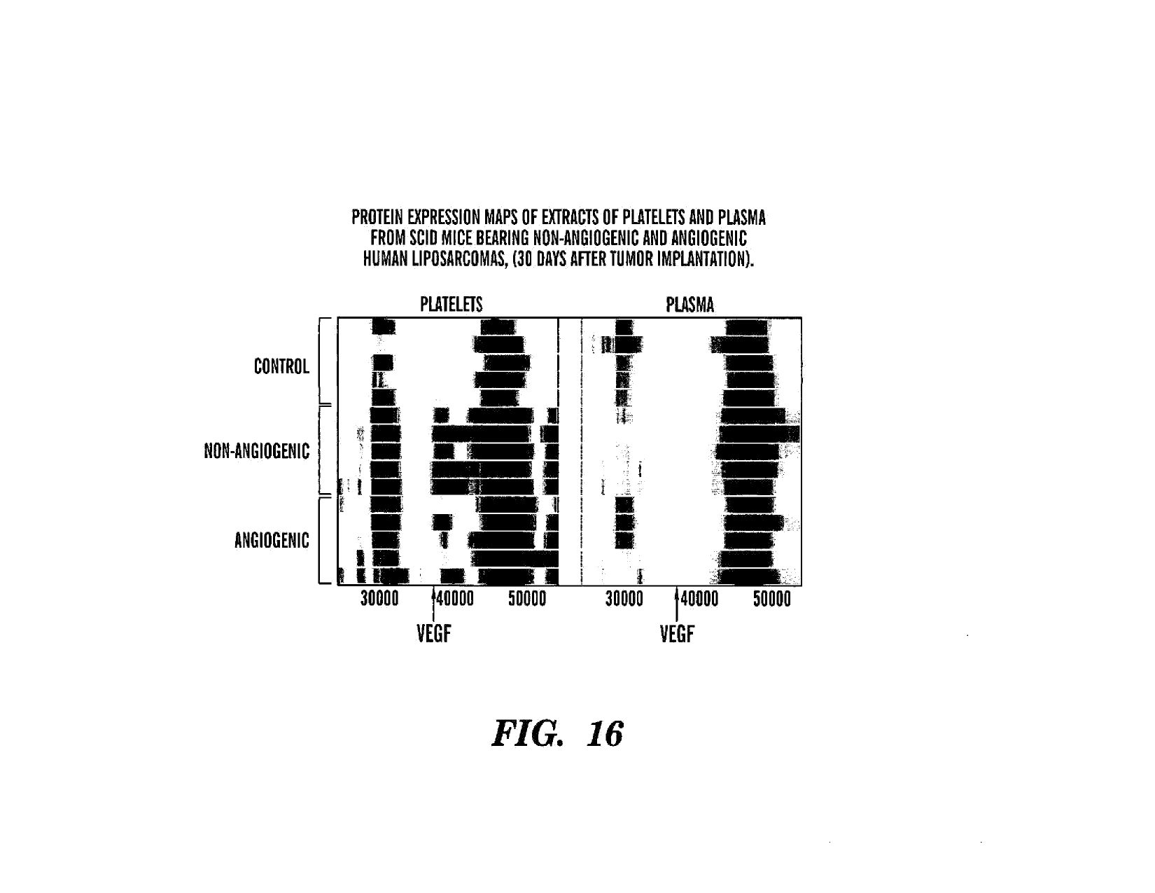

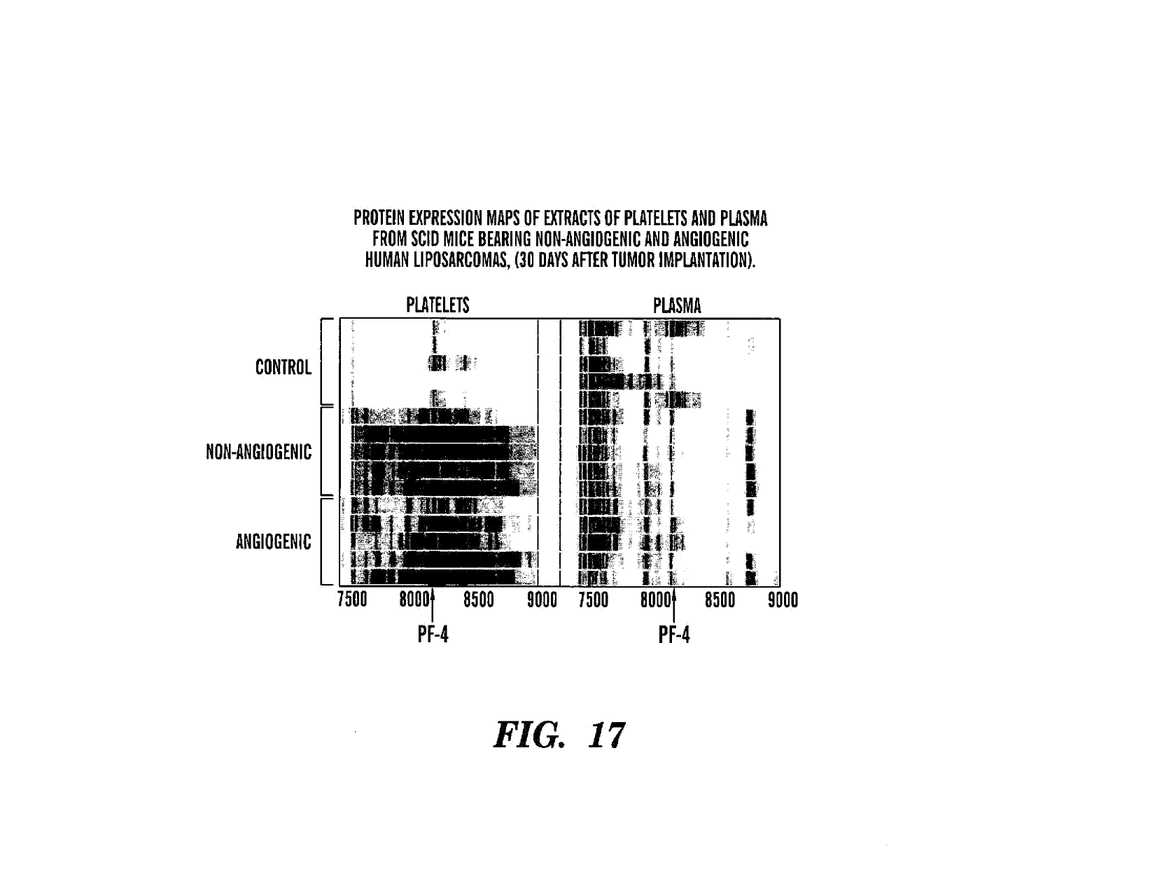

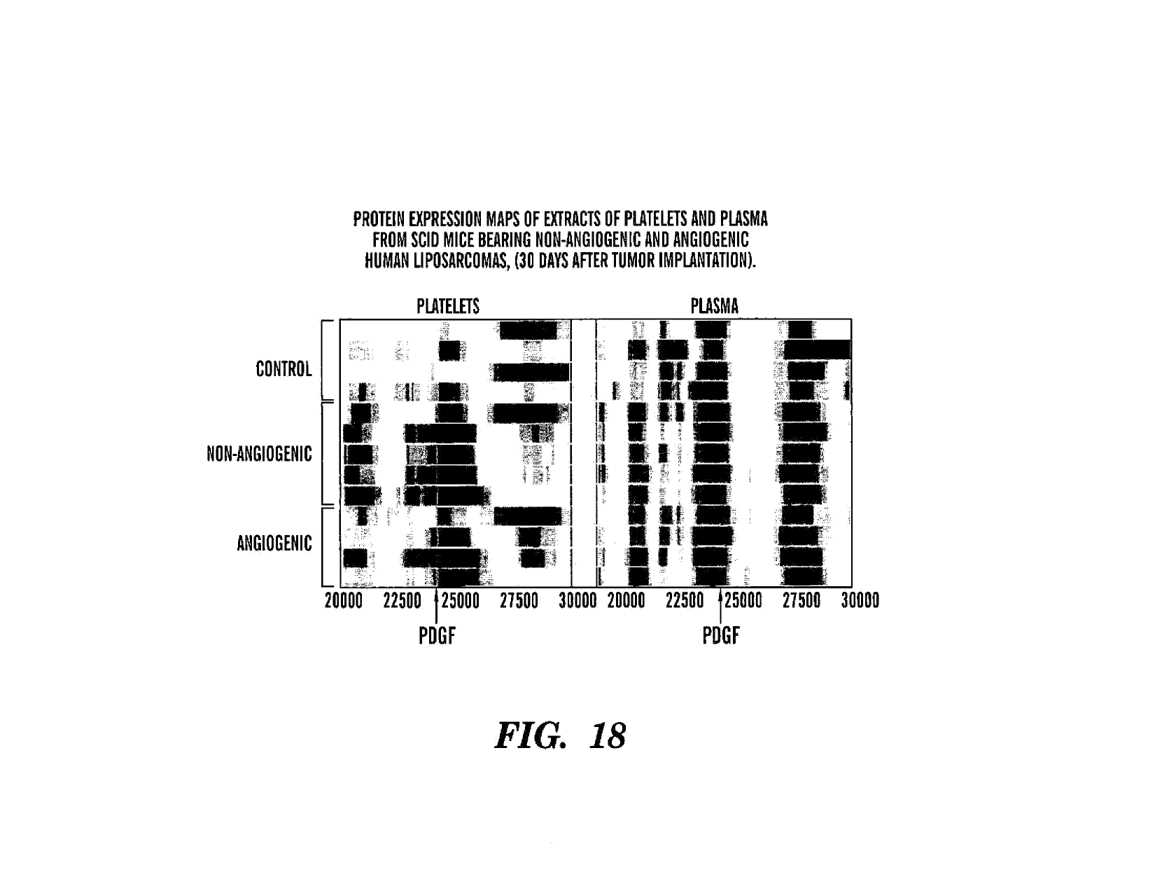

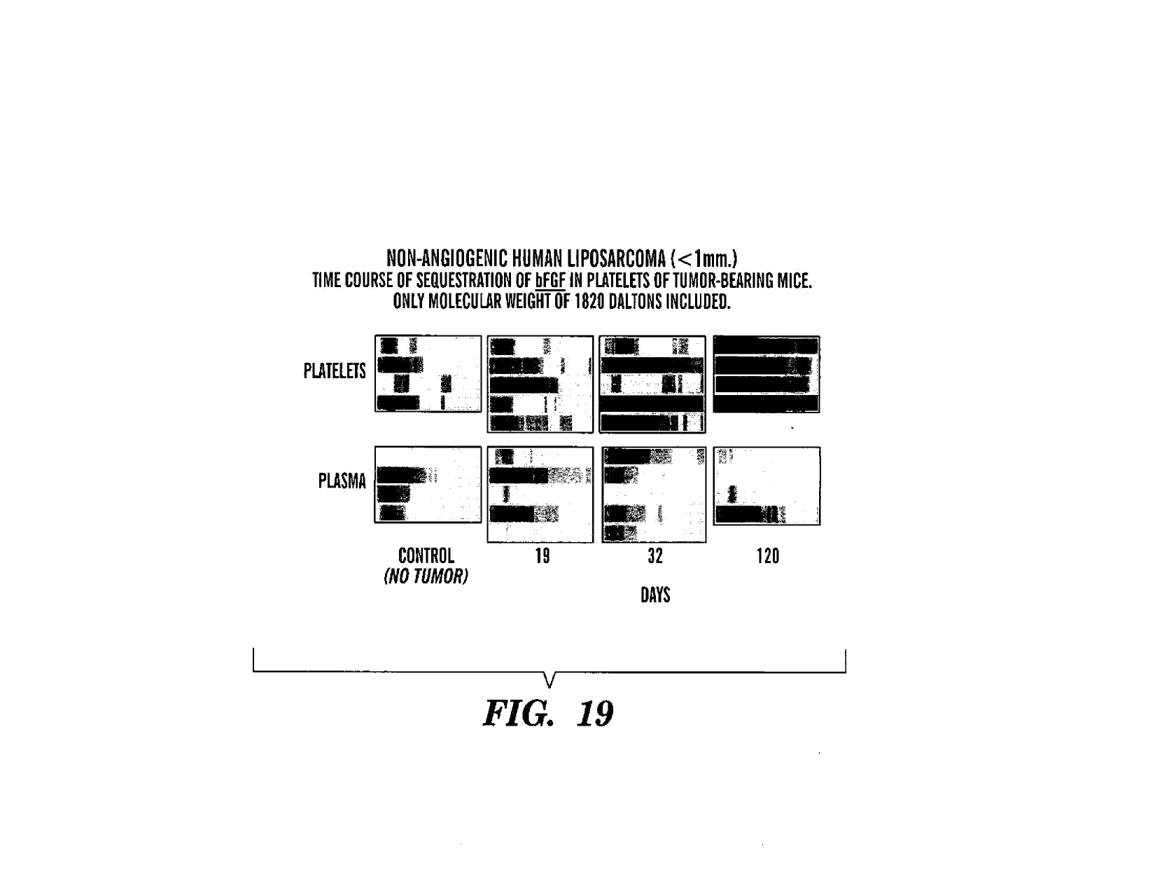

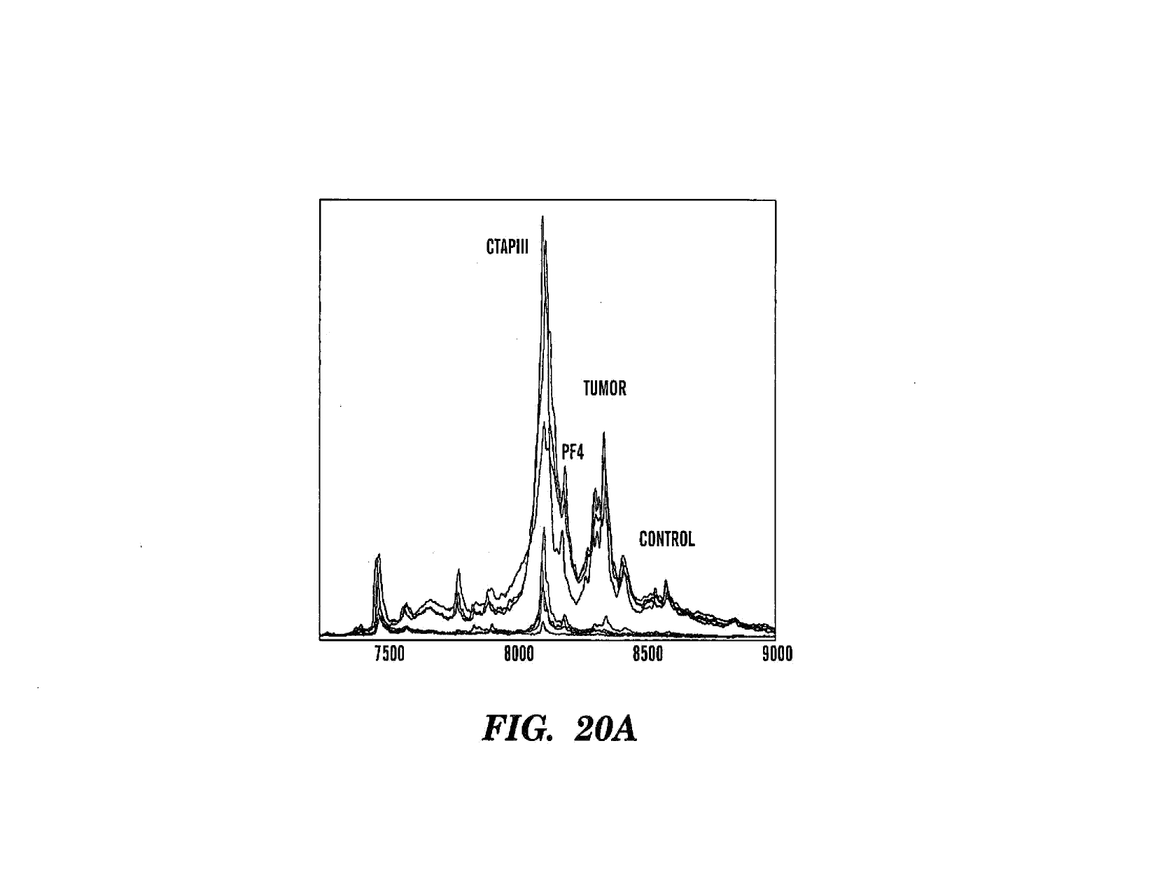

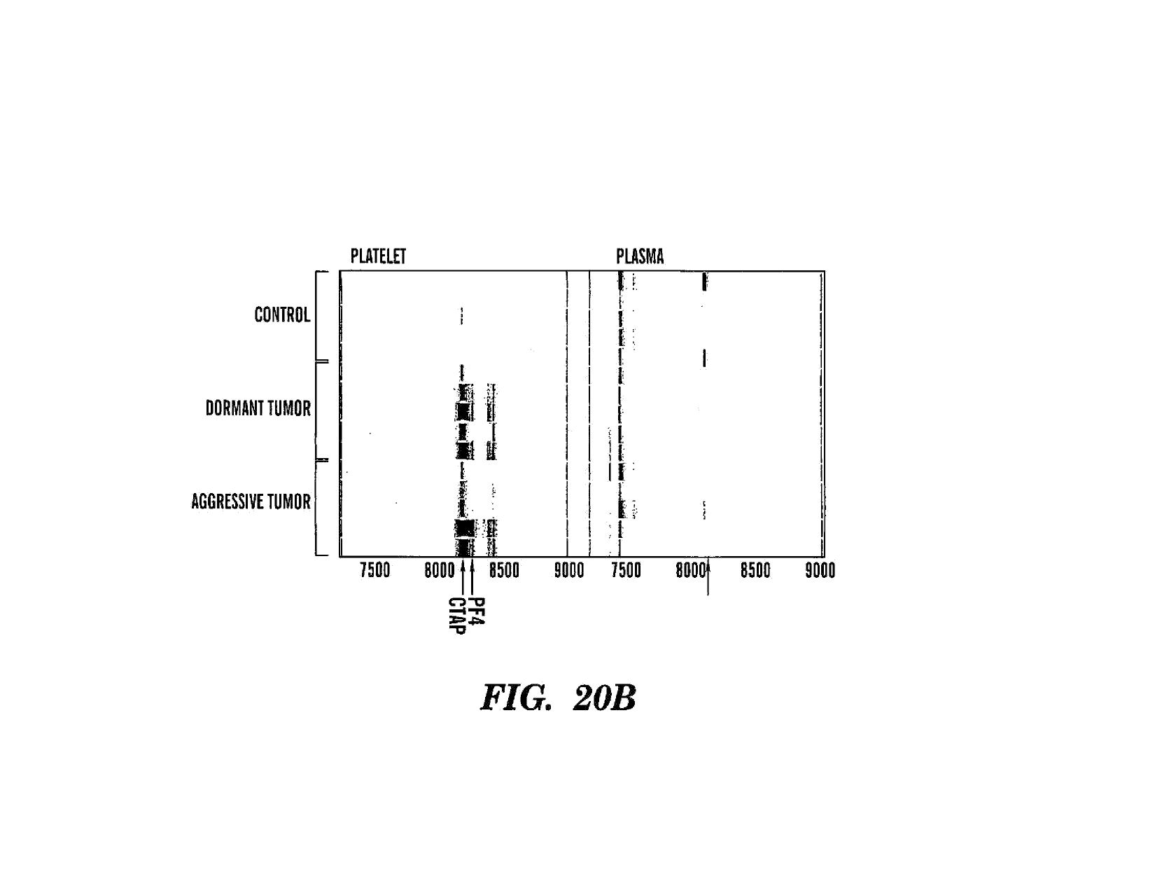

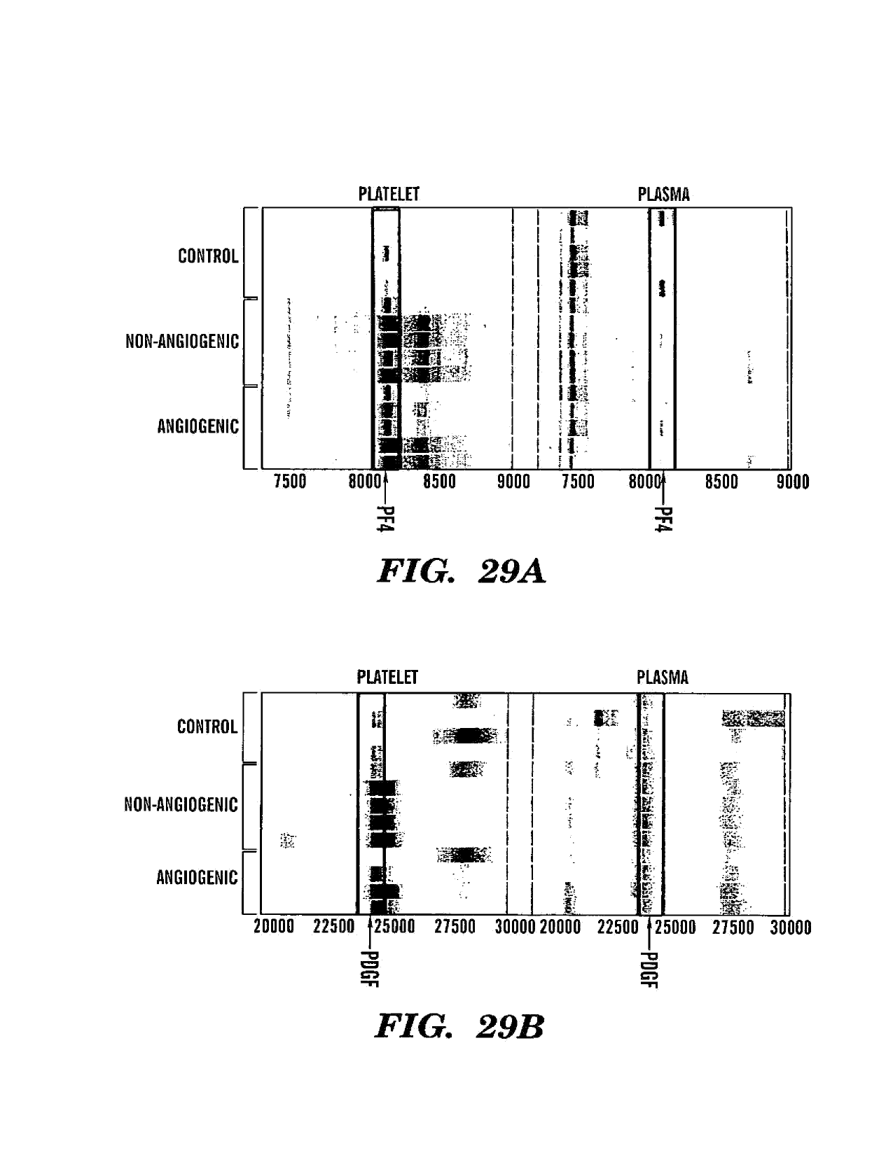

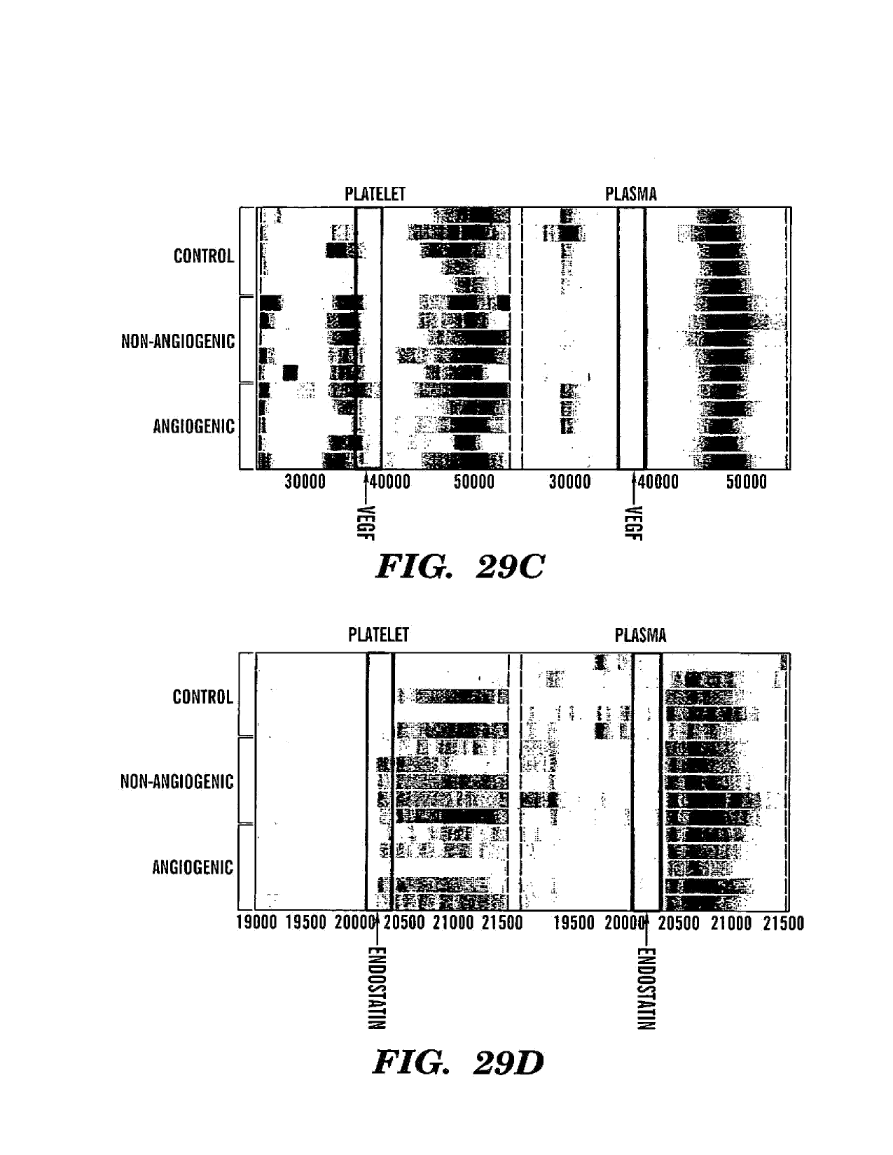

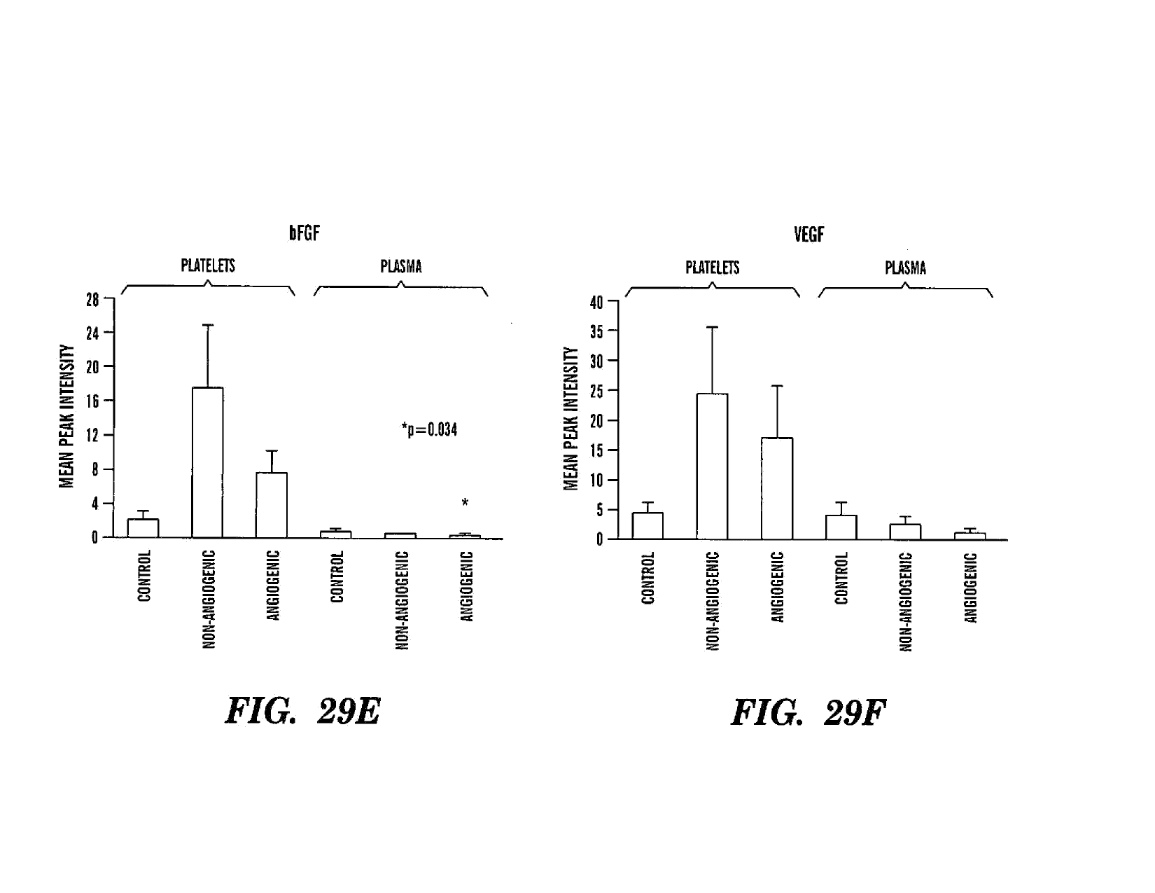

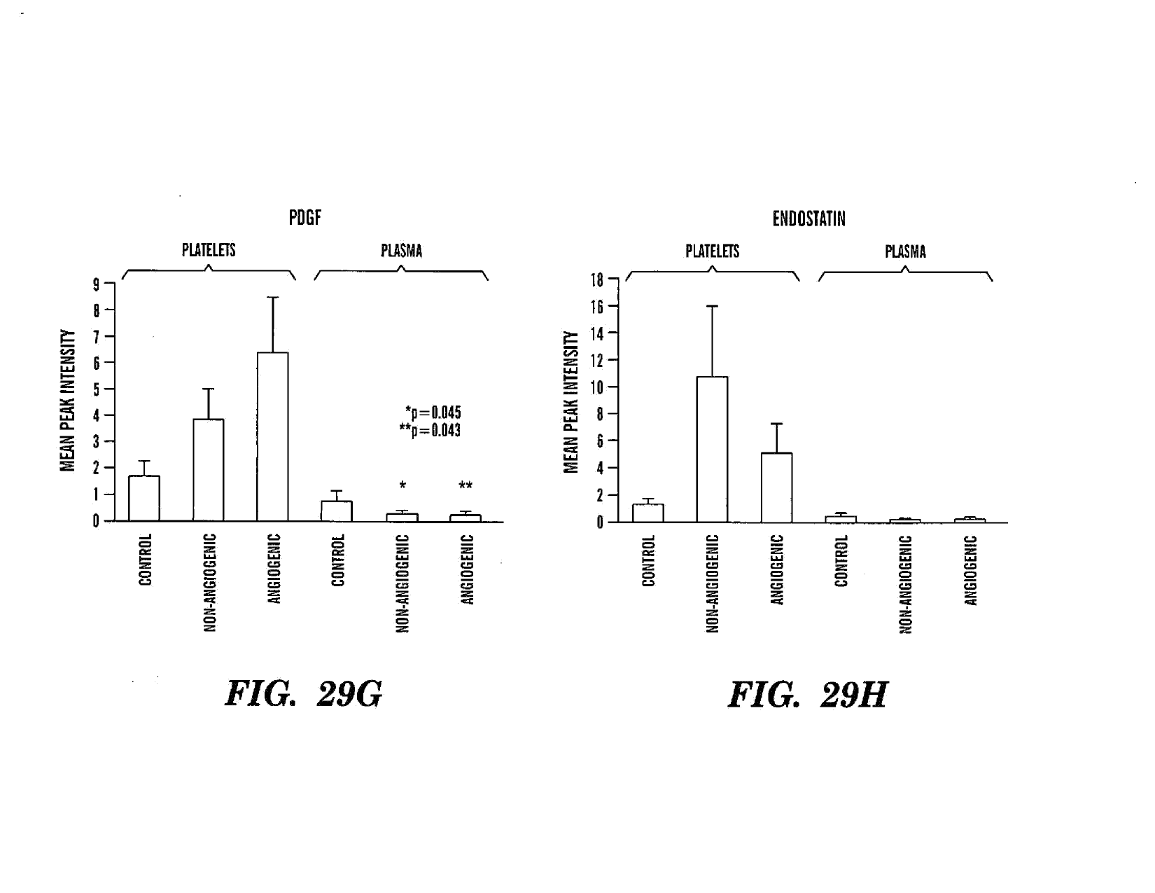

To confirm that the process of protein platelet loading is not an in vitro artifact and to demonstrate that it accurately models an in vivo phenomenon, we implanted Matrigel pellets containing 125I-labelled VEGF (50-600 ng of labeled VEGF per 100 μl of Matrigel) subcutaneously in mice, and followed the uptake of ¹²⁵I-VEGF in platelets (FIG. 12). ¹²⁵I-VEGF accumulated in a dose-dependent manner within platelets preferentially, without any appearance of the labeled cytokine in plasma (FIG. 29). The ¹²⁵I-VEGF was detected in platelets, but not in plasma, for up to three weeks despite the short half-life of murine platelets approximately 4-7 days (data not shown).

Active and Selective Uptake of Angiogenesis Regulatory Proteins by Platelets In Vivo in the Presence of Microscopic Tumors.

To determine whether angiogenesis regulatory proteins secreted by a microscopic tumor in the subcutaneous tissue of mice could be taken up by platelets, analogous to the platelet uptake of VEGF from an implanted Matrigel pellet, subclones of human liposarcoma (SW872) were employed as described above and previously reported. We therefore used an Expression Difference Mapping system (Ciphergen®, Fremont, Calif.) to characterize and validate candidate protein biomarkers at day 32 post tumor implantation. We compared the platelet and plasma proteomes of 5 mice injected with either 200 μl serum free media (vehicle), or a cell suspension of 5×10⁶ cells of the non-angiogenic or angiogenic clones of the liposarcoma cell line. The experiment was repeated twice for comparison of expression maps from separate analyses. (FIG. 29 depicts a typical analysis of a platelet angiogenesis proteome in gel view format, with the respective statistical analysis of the peak intensities). VEGF, bFGF, PDGF, endostatin, angiostatin, tumstatin and other regulators of angiogenesis were significantly increased in platelets from mice bearing non-angiogenic, dormant, microscopic-sized liposarcoma (FIG. 29). The platelets associated proteins were taken up in a selective and quantifiable manner, clearly showing increased concentrations of VEGF, bFGF, PDGF, and platelet factor 4 in the platelet lysate, but not in the corresponding plasma (FIG. 29). Platelets maintain high concentrations of sequestered angiogenesis regulatory proteins platelets for as long as the tumor is present. Despite the fact that at 32 days the angiogenic liposarcoma (˜1 cm³) is ˜100 times larger than the non-angiogenic dormant liposarcoma (<1 mm³), platelets of mice bearing non-angiogenic tumors contain similarly increased levels of angiogenesis regulatory proteins. At this time, the plasma for either tumor type does not contain these proteins. However, in approximately 30 days, with progressive growth of the angiogenic tumor to approximately 2 cm³, the angiogenesis regulatory proteins begin to appear in the plasma fraction as well. In contrast, these proteins never appear in the plasma of mice bearing non-angiogenic microscopic tumors.

The mean peak intensities +/−SE were examined for between group differences using ANOVA. The analysis of the peak intensity values for VEGF, bFGF and PDGF revealed significant differences in the platelet concentrations of these proteins between animals without tumors vs those bearing liposarcoma. Furthermore, platelets from mice bearing non-angiogenic liposarcoma contained high levels of different angiogenesis regulatory proteins than the angiogenesis regulatory proteins accumulated in platelets from mice bearing human breast cancer.

VEGF Distribution in Platelets.

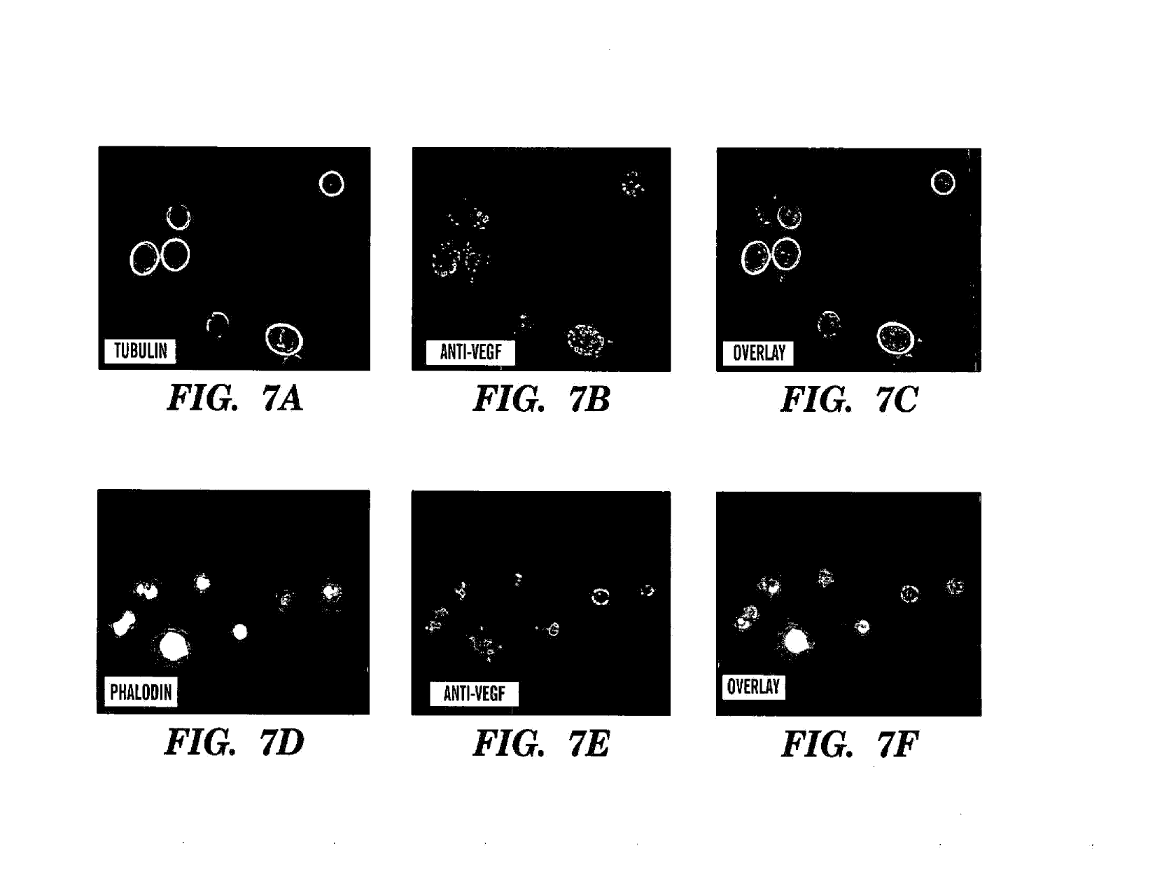

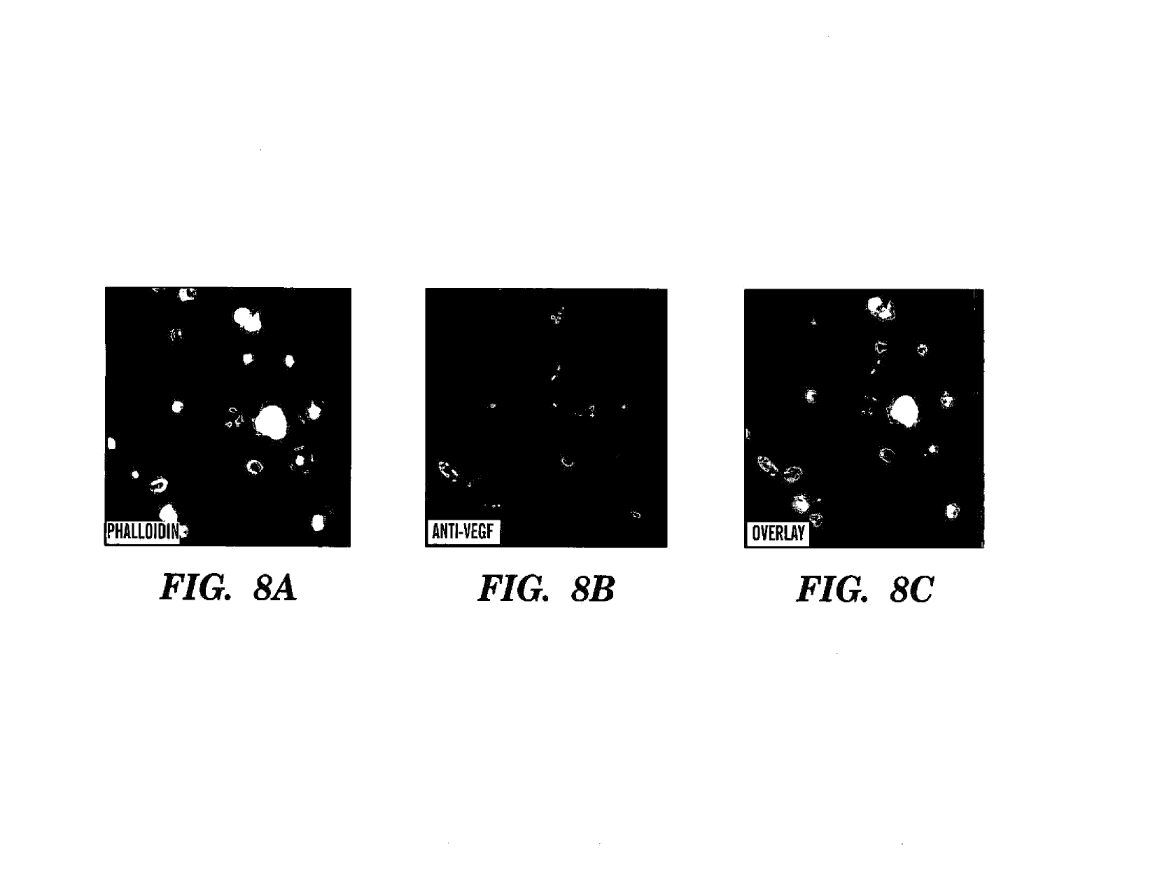

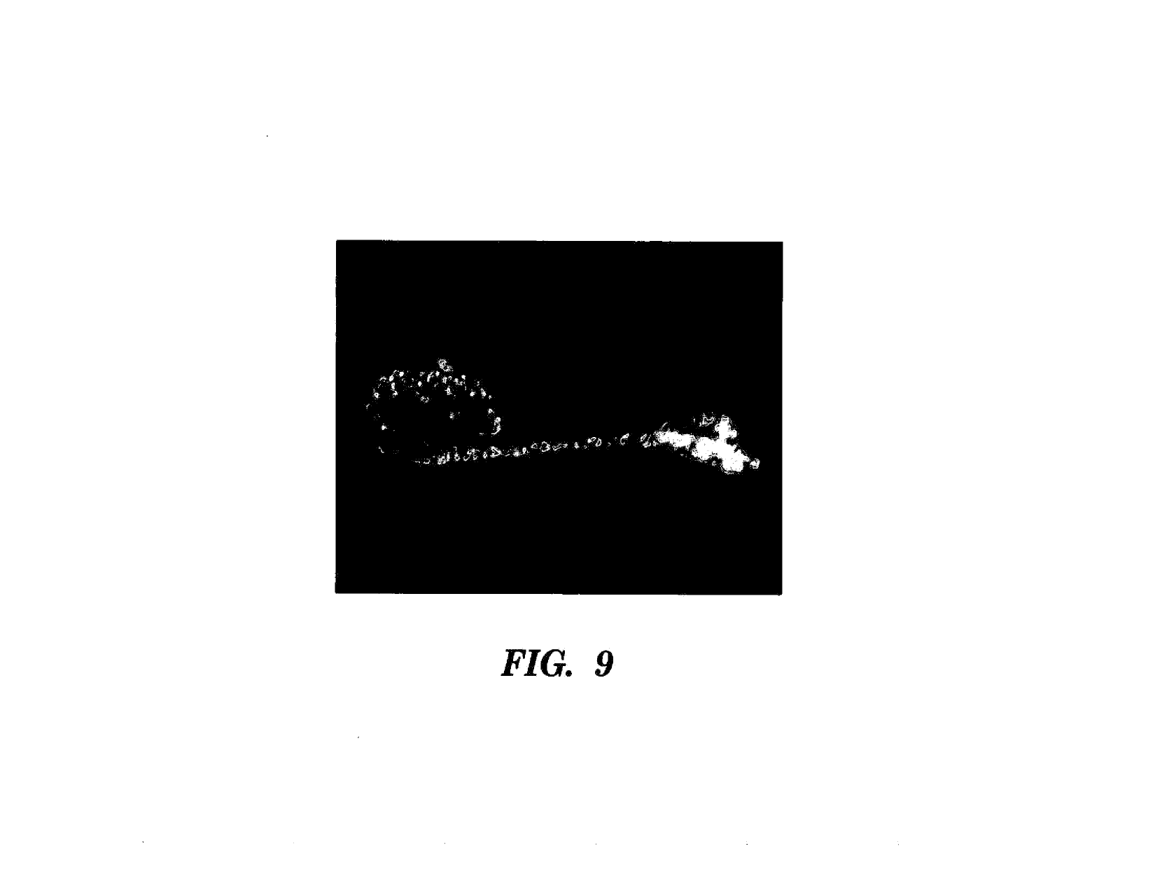

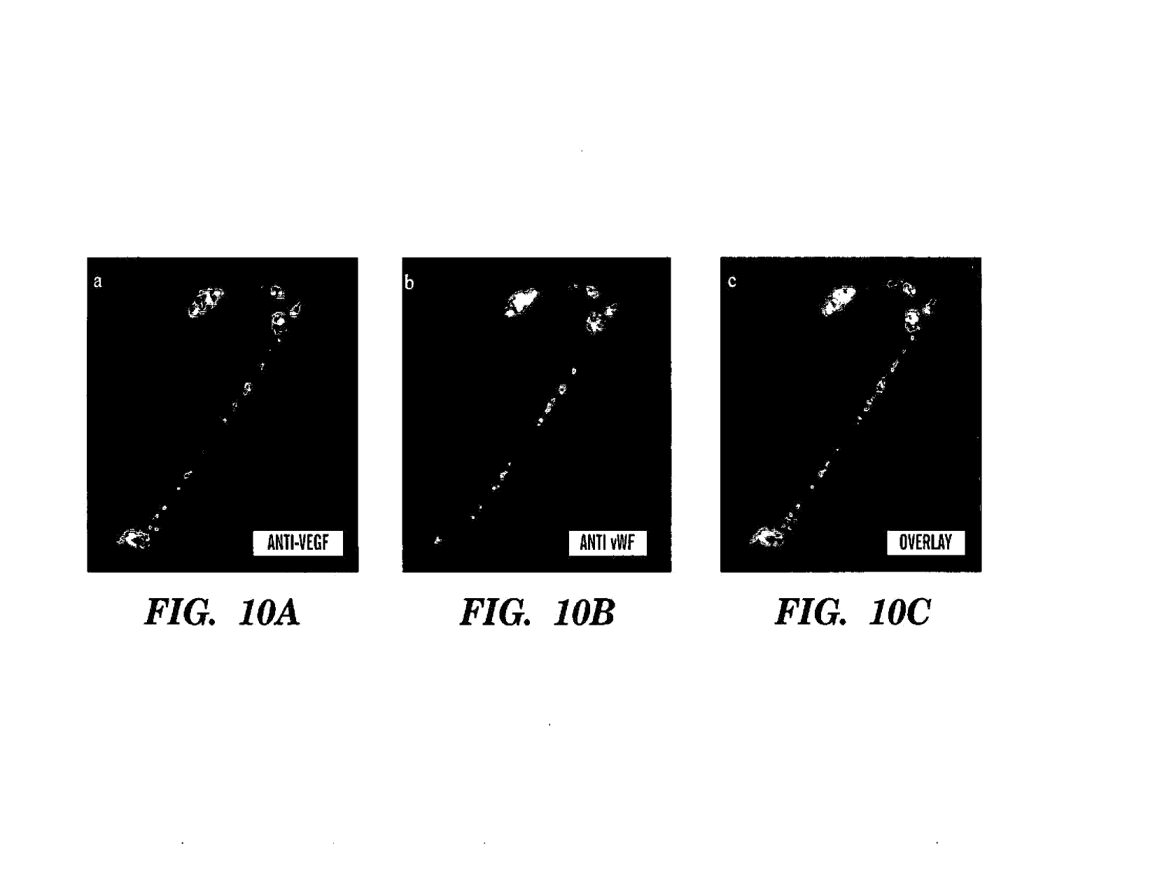

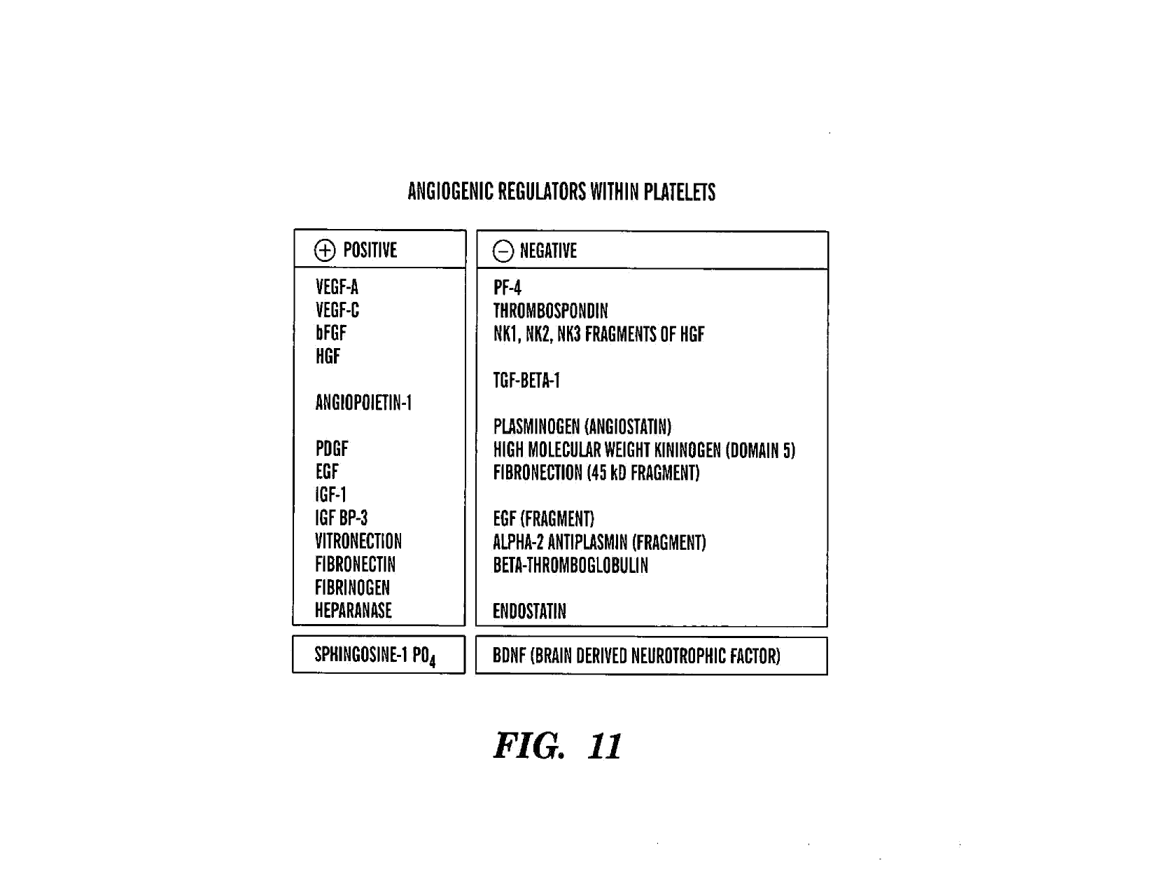

At the beginning of this study it was unclear whether the angiogenesis regulatory proteins associated with platelets were distributed uniformly on the membrane of platelets, or throughout the cytoplasm of the platelet body, or whether they were organized in specific granular stores. To distinguish between these possibilities and to establish the subcellular localization of VEGF, we used double label immunofluorescence microscopy on fixed and permeabilized resting platelets stained with antibodies for tubulin and VEGF. As expected, tubulin was concentrated in the marginal microtubule band in a resting platelet and this structure defined the platelet periphery. However, anti-VEGF antibodies consistently labeled punctate, vesicle-like structures distributed throughout the platelet cytoplasm (FIG. 7, A-C). Sequential stacking of 4 μm slices of confocal microscope images revealed the punctate pattern within the cytoplasm of the platelet and supported a granular nature of the immunoreactive material.

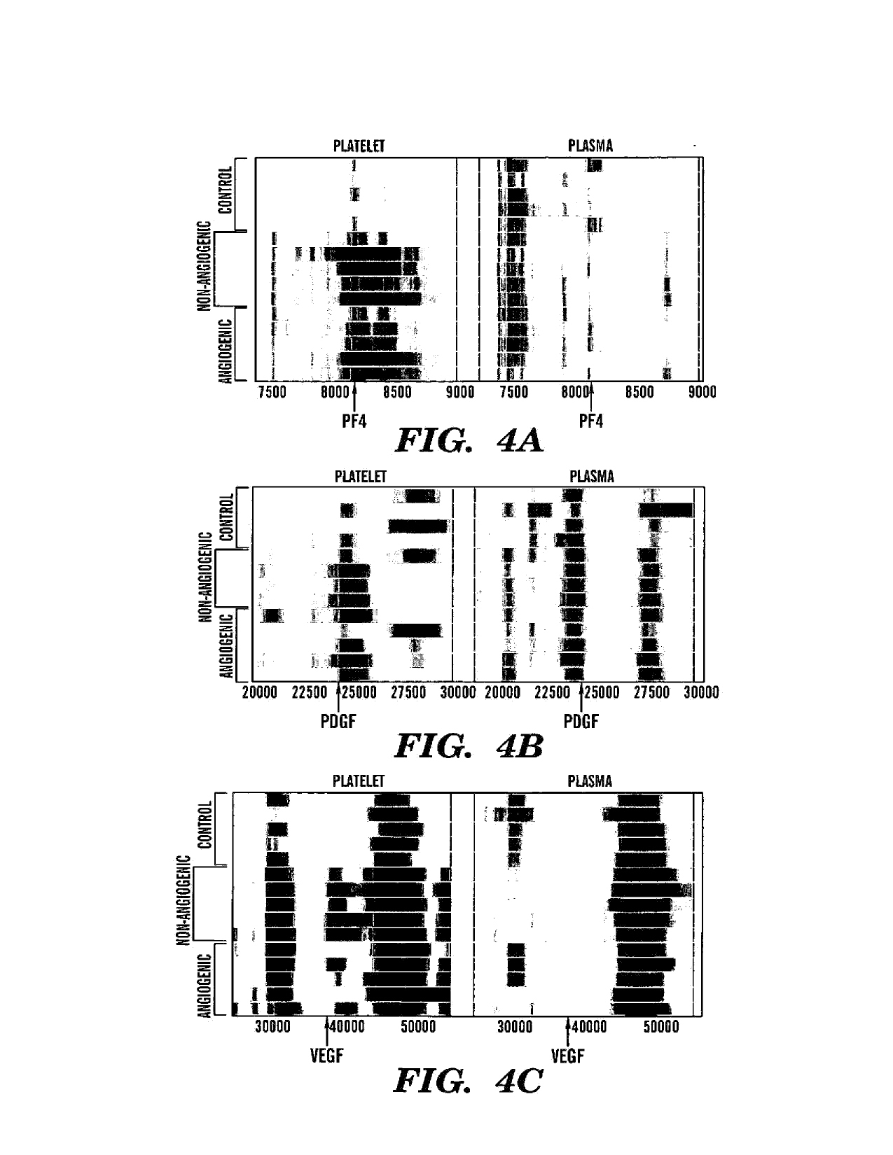

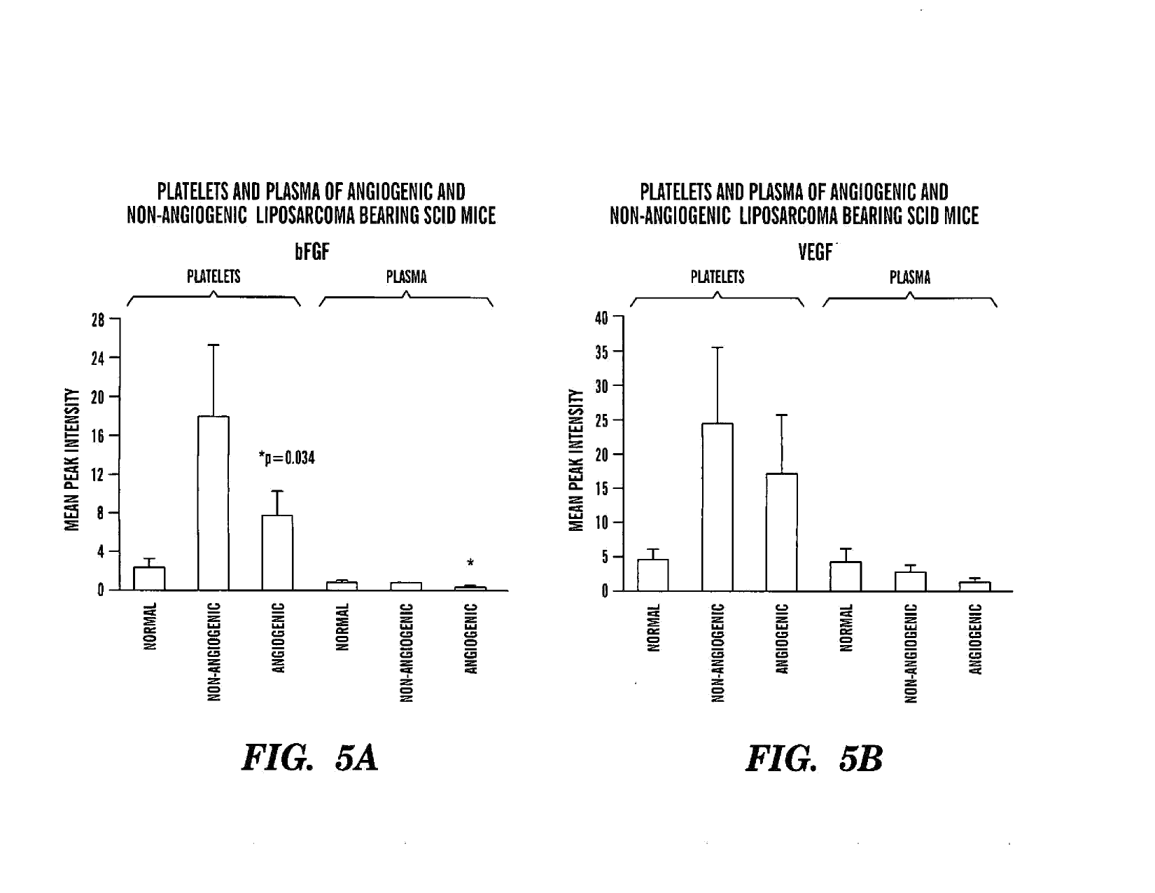

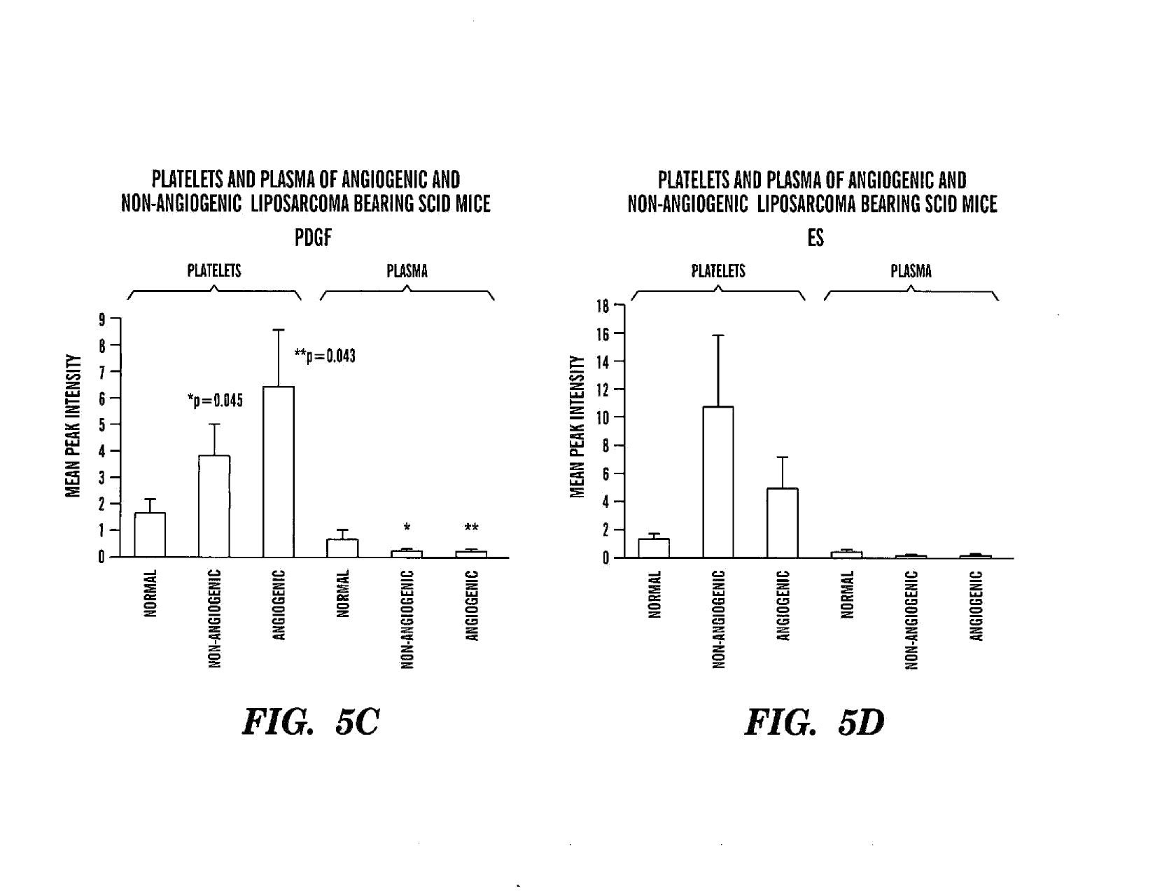

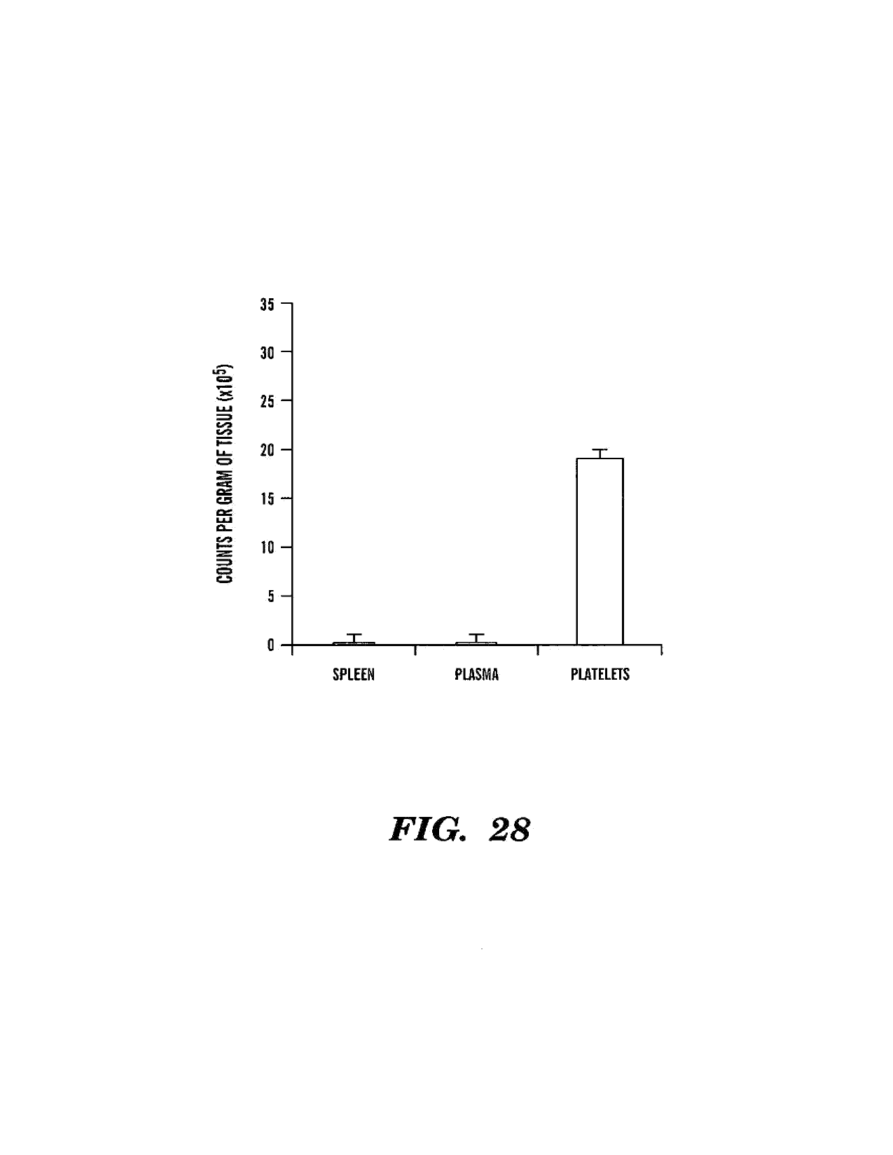

Our analysis of the release from platelets activated with thrombin suggests that VEGF is not released into the plasma by the loading of platelets with endostatin (FIG. 28) or with mild activators such as ADP (FIG. 4). Thrombin, but not ADP was able to release some, but not all of the platelet associated VEGF (FIG. 4, upper panel). Neither activator (thrombin or ADP) was able to liberate bFGF. We therefore hypothesize, that during agonist induced platelet activation, platelet associated growth factors are re-distributed, but continue to be retained within platelets. To test this concept, we double stained activated platelets with fluorescently-labeled phalloidin and VEGF. The expected platelet-shape change was clearly documented by the formation of lamelipodia and filopodia. VEGF remained observable as punctate patterns in activated, spread platelets, consistent with the notion that it remains associated with platelets even after agonist-induced activation (FIG. 7, F). Upon platelet activation, VEGF appeared to be preferentially re-distributed along the filopodia and along the periphery of lamellipodia.

Discussion:

These results show that circulating platelets take up angiogenesis regulatory proteins produced by human tumors in mice. The proteins taken up under these conditions are essentially the same as the approximately 30 angiogenesis regulatory proteins contained in normal platelets i.e., bFGF, VEGF, endostatin, angiostatin and others. We have named this select group of proteins the “platelet angiogenesis proteome” to emphasize the stability of the relative protein concentrations under physiological conditions. Under normal conditions, the membership in this proteome appears to vary very little. However, in a tumor-bearing mouse, the tumor-induced uptake of angiogenesis regulatory proteins significantly alters the platelet angiogenesis proteome, and the increased concentrations of a sequestered tumor-derived angiogenesis regulatory protein (i.e., VEGF, or bFGF etc), remain elevated as long as there is a viable tumor in the host.

Circulating platelets can take up and sequester angiogenesis regulatory proteins released from a small tumor mass, i.e., cancers smaller than 1 mm³ This is equivalent to less than 1 milligram of tumor mass in a host mouse that weighs more than 20,000 milligrams. Tumors of this minute size cannot be, at least at present, detected clinically. Experimentally it can be identified using bioluminescence, i.e., using tumor cells transfected before implantation with the gene for green fluorescent protein, or infected with luciferase. These tumors develop from subcutaneous or orthotopic implantation of cloned non-angiogenic human cancer cells, and can be exposed surgically under stereoscopic magnification. They remain dormant and harmless for months, or for more than a year, until a predictable percentage of them, at a predictable time, switch to the angiogenic phenotype and begin to grow at rates very comparable to their angiogenic counterparts. The non-angiogenic tumors never spontaneously metastasize, although the tumor cells within them if injected into the tail vein will form microscopic, benign, dormant metastases in the lung. In contrast, after the angiogenic switch spontaneous metastasis is not uncommon (20). The tumor cells in these non-angiogenic dormant tumors are undergoing a high rate of proliferation which is balanced by a high rate of apoptosis, which is in agreement with our finding of increased endostatin levels in the non-angiogenic clone as compared with its angiogenic counterpart (FIG. 30). Tumor dormancy due to blocked angiogenesis has been previously described (21, 22).

The angiogenesis regulatory proteins secreted by non-angiogenic microscopic tumors are sequestered in platelets, do not appear in the plasma, and continue to be added to the basal level of proteins in the platelet angiogenesis proteome for as long as the tumor is present. When a non-angiogenic tumor switches to the angiogenic phenotype and begins to expand its tumor mass, angiogenesis regulatory proteins secreted by the tumor may appear in the plasma as well.

The platelet sequestration of tumor-derived angiogenesis regulatory proteins involves a process by which these proteins are internalized by circulating platelets and re-distributed to different compartments within in the platelets by mechanisms which remain to be elucidated. The platelet storage compartments consist of α-granules, dense granules and lysosomes, with α-granules forming the largest compartment. Many platelet proteins are synthesized in megakaryocytes, others are clearly picked up in the periphery. Platelet-specific proteins such as PF4 and thrombomodulin are synthesized by a number of cells including megakaryocytes and concentrate in platelets in 400 fold concentrations. Others such as Factor V, thrombospondin or P-selectin are synthesized by non megakaryocytes and taken up by platelets. The most notably platelet nonselective protein is fibrinogen, which is synthesized by the hepatocytes and taken up by platelet α-granules (14-16). This remarkable flexibility of the platelet storage compartment led us to believe that platelets are involved in the amplification and maintenance of tumors. We found that large concentrations of VEGF, bFGF or endostatin could be taken up, internalized and concentrated in platelets. Fresh platelets exposed to increasing concentrations of Endostatin, displace endogenous growth factors such as bFGF and VEGF from their cytoplasmic storage (FIG. 1), suggesting a fluid, highly adaptable trafficking of these proteins.

At least two possibilities exist for the regulation of platelet uptake of proteins. The storage compartment of platelets may be of limited capacity and proteins must be displaced to accommodate the uptake of new ones, or, more likely, the uptake is governed by specific platelet regulated affinity for the factor. The latter model would be more consistent with the finding that sequential loading of platelets results in selective uptake, and that not all proteins are displaced with equal efficiency. While the uptake of Endostatin into platelets pre-loaded with VEGF was full, unencumbered (first lane of FIG. 2), and resulted in re-distribution of the pre-loaded VEGF (second lane of FIG. 2), the opposite experiment, resulted in an incomplete re-distribution of the pre-loaded Endostatin.

An important finding was the relative absence of angiogenesis regulatory proteins in the plasma of tumor-bearing mice. This most common clinical analyte showed minimal or no differences in angiogenic proteins. This suggested that, contrary to commonly held beliefs, platelets may not undergo degranulation with a uncontrolled release of the growth regulators into circulation, but rather liberate these regulators under strict control at the tumor or wound site. This was consistent with our in vitro analysis of the release rate from activated platelets using VEGF and bFGF ELISA, where minimal amounts of VEGF were released during agonist induced platelet activation, and bFGF was not released at all (FIG. 28).

If so, we predicted that significant amounts of VEGF would remain localized to activated platelets following activation. Using double label immunofluorescence microscopy with antibodies against tubulin and VEGF for fixed and permeabilized resting platelets; and phaloidin and VEGF for activated platelets, we examined the intracellular location of VEGF. The anti-VEGF antibodies consistently labeled punctate, vesicle-like structures distributed throughout the cytoplasm of resting platelets, suggesting a granular nature of the immunoreactive material. On double label immunofluorescence of activated platelets, VEGF was re-distributed along the filopodia and along the periphery of lamellipodia (FIG. 7), consistent with the notion that it remains associated with platelets even after agonist-induced activation. Platelet-shape change was clearly documented by the formation of lamellipodia and filopodia, and visualized by fluorescent phalloidin. This pattern of redistribution points out the possibility that VEGF marginates within platelets for a direct exchange of these proteins with the tissues, and may explain the induction of tissue proteases in tumors. It is not clear yet which specific proteases would act to liberate angiogenic regulators from tumor-associated platelet aggregates.

We have shown that the process of platelet uptake of angiogenesis regulators is highly specific, reflects the tumor status, i.e dormancy vs clinical expansion, and occurs well in advance of clinically detectable tumors. We propose that this novel compartment in the systemic circulation is superior to plasma and serum analysis of angiogenic markers, and provides a stable, sensitive and reliable method for very early cancer diagnosis. A “platelet angiogenesis proteome” may be used as an early register of tumor angiogenic switch, in much the same way that a lipid profile is used to identify patients at risk for artherosclerosis and myocardial infarction. This forecasting biomarker may be to screen patients at risk for developing cancer. Used in conjunction with other biomarkers (23) we may be able to diagnose cancer recurrence years in advance of clinical symptoms, or improve the monitoring of women with BRCA cancer gene mutation and at high risk of developing breast cancer.

The recent development of relatively non-toxic angiogenesis inhibitors may provide us with an opportunity to “treat a biomarker” without ever “seeing” the tumor, in other words, treat a patient who has cancer without disease (24). Several angiogenesis inhibitors are now approved in the U.S., and in 27 other countries, and others are in late phase clinical trials. Analogies in medical practice in which biomarkers in the blood or urine guide therapy without the necessity of anatomical location include the treatment of suspected infection or the use of lipid lowering agents to prevent future myocardial infarction.

The results reported here uncover new platelet biology which has implications for reproduction, development, repair, and for understanding of the many angiogenesis-dependent diseases.

Following a disruption of the endothelial cell lining of a blood vessel by either tumor or trauma, circulating platelets function to localize, amplify and sustain to pro-coagulant response at the site. Platelet adherence and aggregation at the site of vascular injury serves not only to temporary plug the damaged vessel, but also to localize subsequent pro-coagulant events to the injury site and prevent systemic activation of coagulation. Interestingly, we find the same localization serves to localize, amplify and sustain angiogenic stimulus at the site of the tumor.

The platelet storage compartments consist of α granules, dense granules and lysosomes, with α granules forming the largest compartment. The stored proteins are either synthesized in megakaryocytes (platelet specific proteins such as PF4 and thrombomodulin), synthesized by a number of cells including megakaryocytes and concentrated in platelets in up to 400 fold concentrations (platelets selective proteins such as Factor V, thrombospondin or P selectin), or synthesized by other cells and taken up by platelets (platelet nonselective proteins such as fibrinogen (14-16)). It is the remarkable flexibility of this later compartment that led us to believe platelets may be involved in the amplification and maintenance of tumors early in tumor progression before the cancers are clinically evident.

We first tested this hypothesis by “loading” platelets with angiogenic regulators ex-vivo, and found that large concentrations of VEGF, bFGF or Endostatin can be uptaken, internalized and concentrated in platelets. By assaying only the cytoplasmic portion of fresh platelets exposed for one hour to supraphysiologic levels of Endostatin by SDS-PAGE, we found that as the concentration of Endostatin in platelets increased, the levels of endogenous growth factors such as bFGF and VEGF decreased (FIG. 1). We made every effort to eliminate the chance that the increase in Endostatin level in the platelet lysate was due to nonspecific binding to the platelet surface by excluding the membrane portion. We postulated that at least two possibilities existed for the regulation of platelet uptake of these proteins. The storage compartment of platelets may have been of limited capacity and some proteins had to be displaced in order for the new proteins to be uptaken, or the uptake was governed by some specific platelet regulated affinity. It appears that the later, a more selective mechanism, may be the more likely process because sequential loading of platelets with proteins did not appear to be affected by the concentration gradient of the protein and because not all proteins were replaced with equal efficiency. For example, the uptake of Endostatin into platelets pre-loaded with VEGF was not only full, unencumbered, and enhanced in comparison to the Endostatin loading control (first lane of FIG. 2), but also resulted in complete displacement of the pre-loaded VEGF (second lane of FIG. 2). The opposite experiment, i.e. the loading of VEGF into platelets preloaded with Endostatin, was also enhanced in comparison with control, but resulted in a much less complete displacement of the pre-loaded Endostatin.

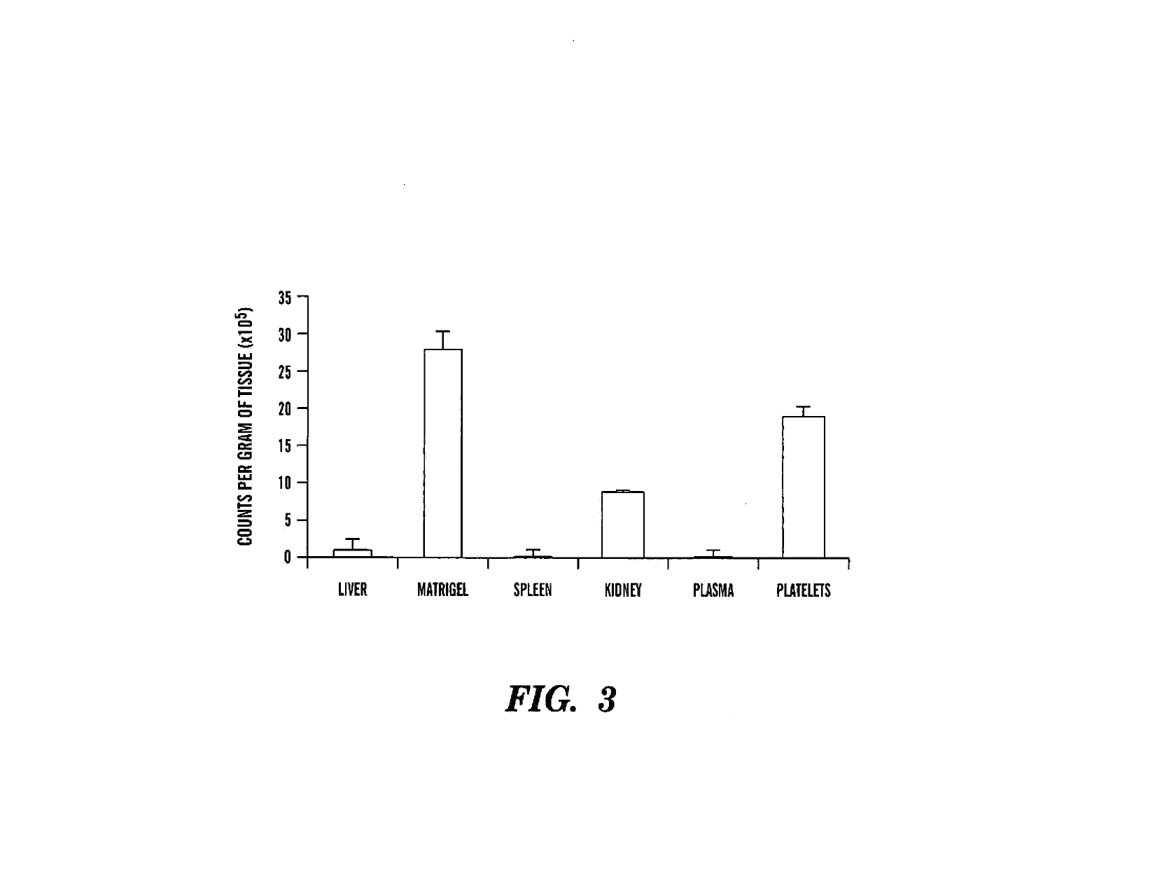

We then went on to confirm this selective uptake of growth factors by platelets in an in vivo model. We implanted a ¹²⁵I labeled VEGF enriched growth factor free Matrigel pellet into healthy, otherwise untreated, immunocompetent mice and compared the distribution of ¹²⁵I in highly vascular organs such as kidney, spleen and liver, which are known to express VEGF constitutively, with its distribution in blood. As seen in FIG. 3 the iodinated VEGF concentrated in platelets in many fold excess of its concentration in plasma. This suggested that platelets may be indeed contributing to the local enhancement of angiogenesis by delivering angiogenic regulators. This delivery would likely be different in physiological situations such as trauma and tissue repair where intact feedback mechanisms act quickly to deactivate functional angiogenesis, and in tumorigenesis where after the transformation the proangiogenic stimulus persists.

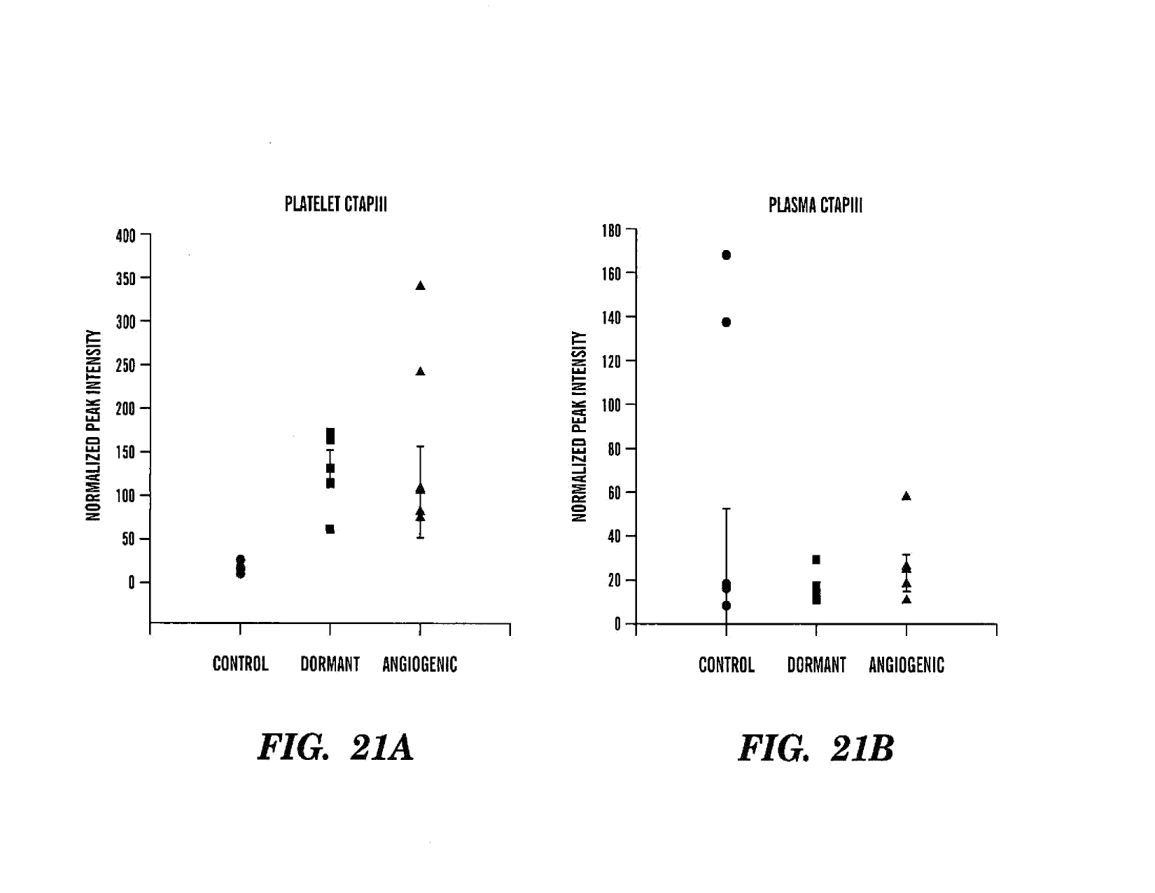

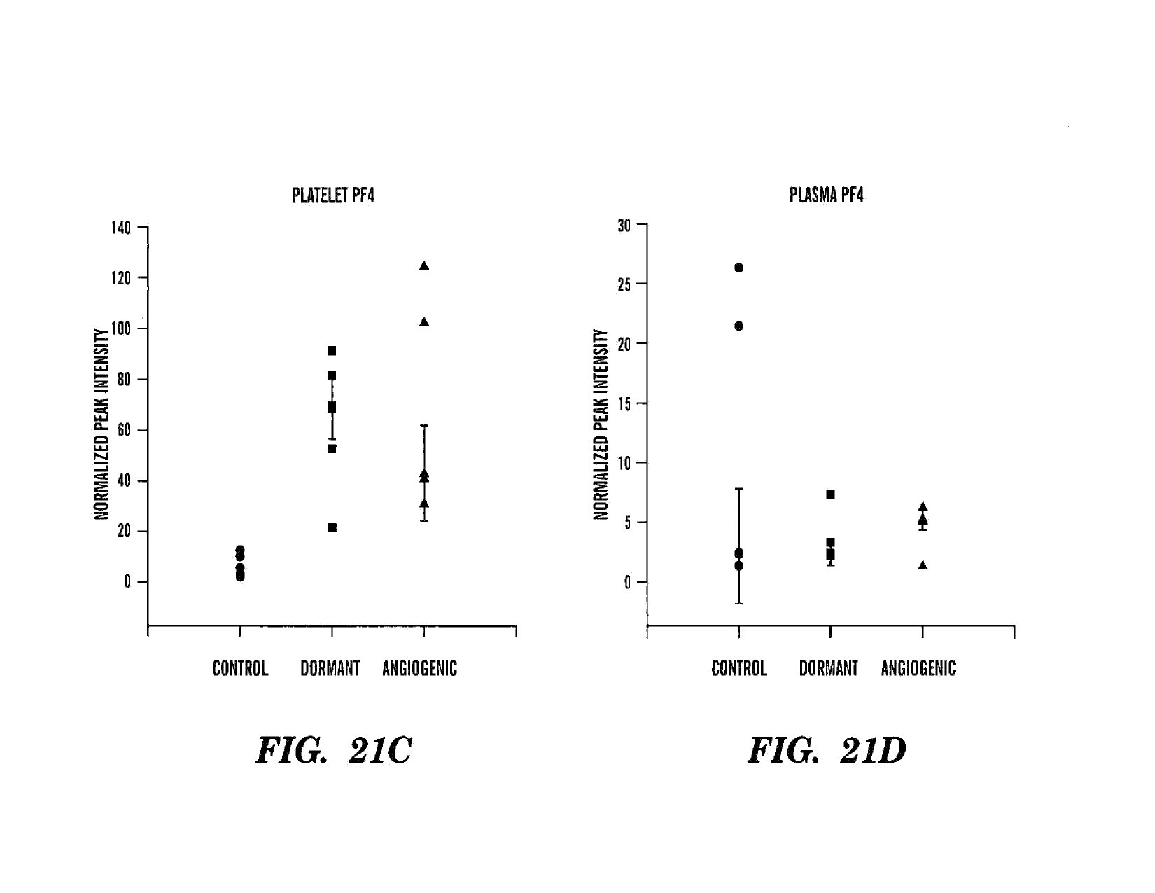

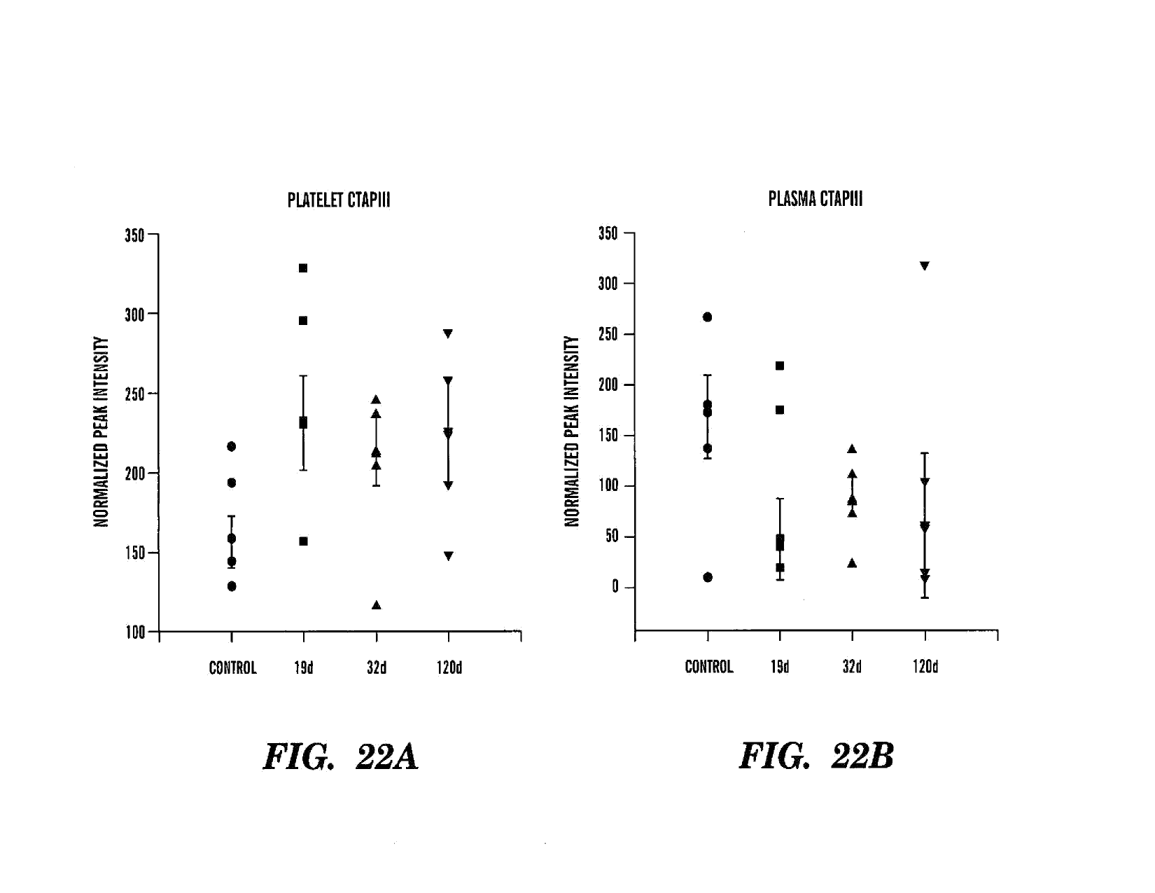

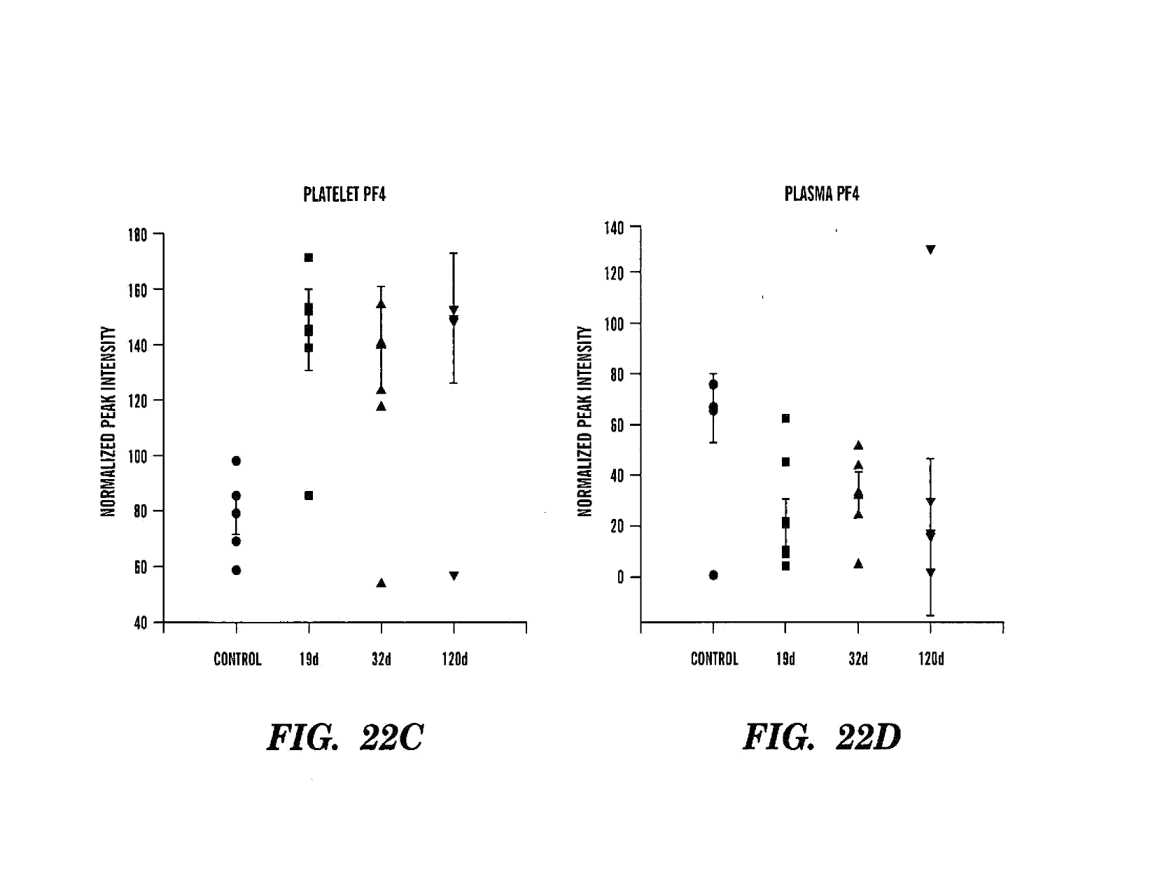

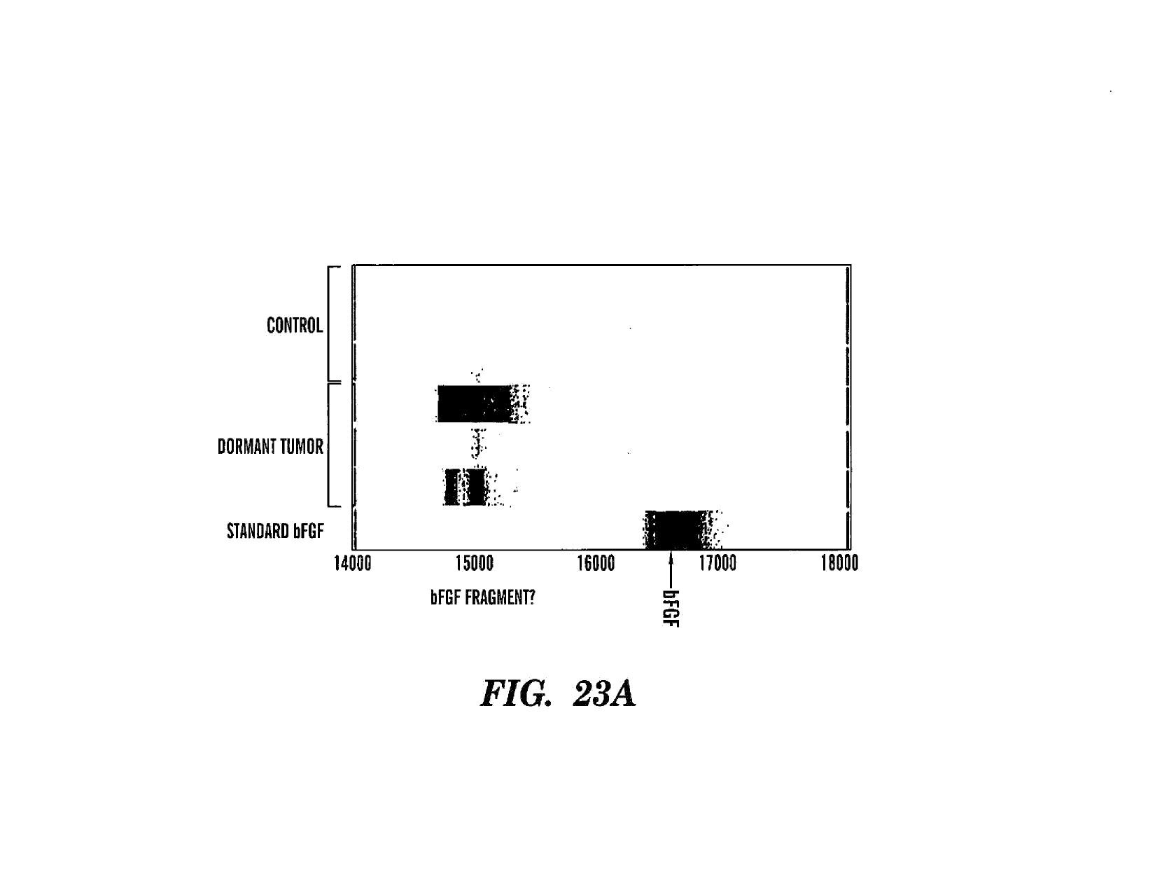

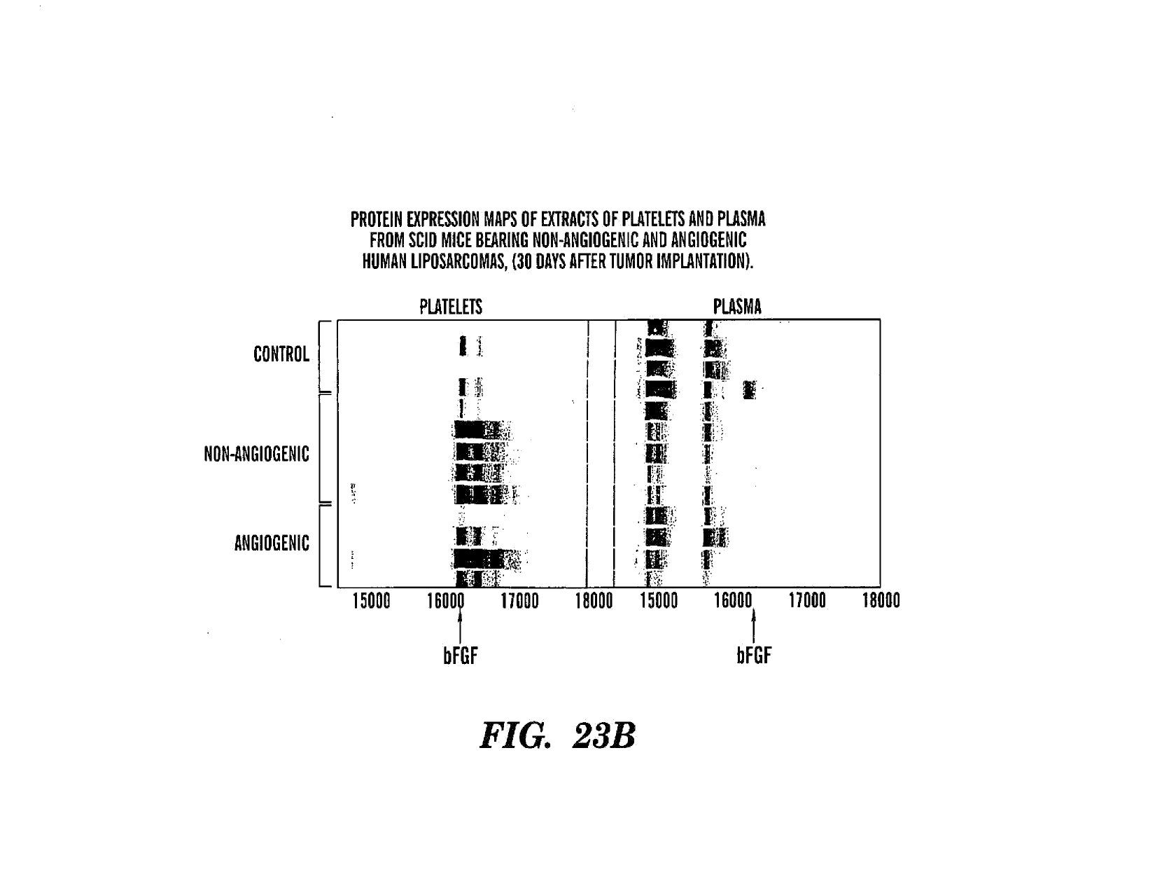

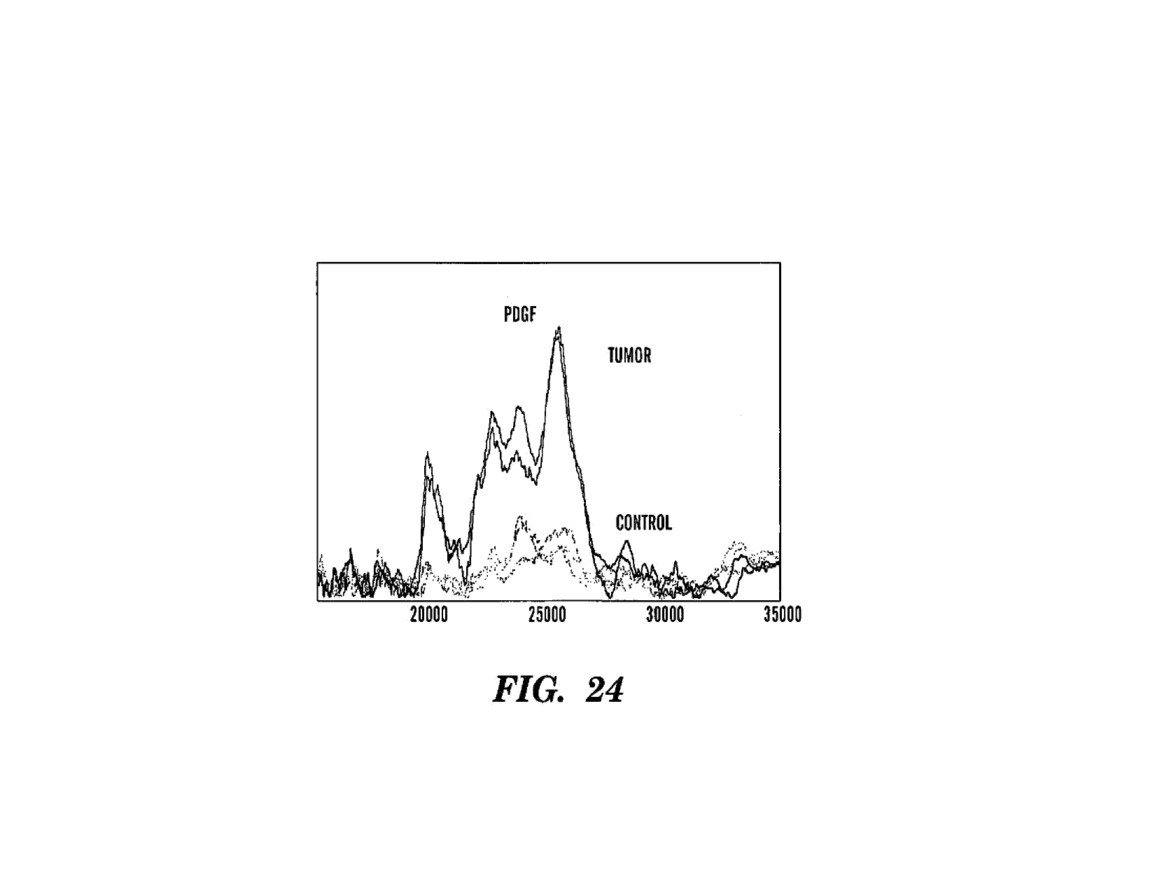

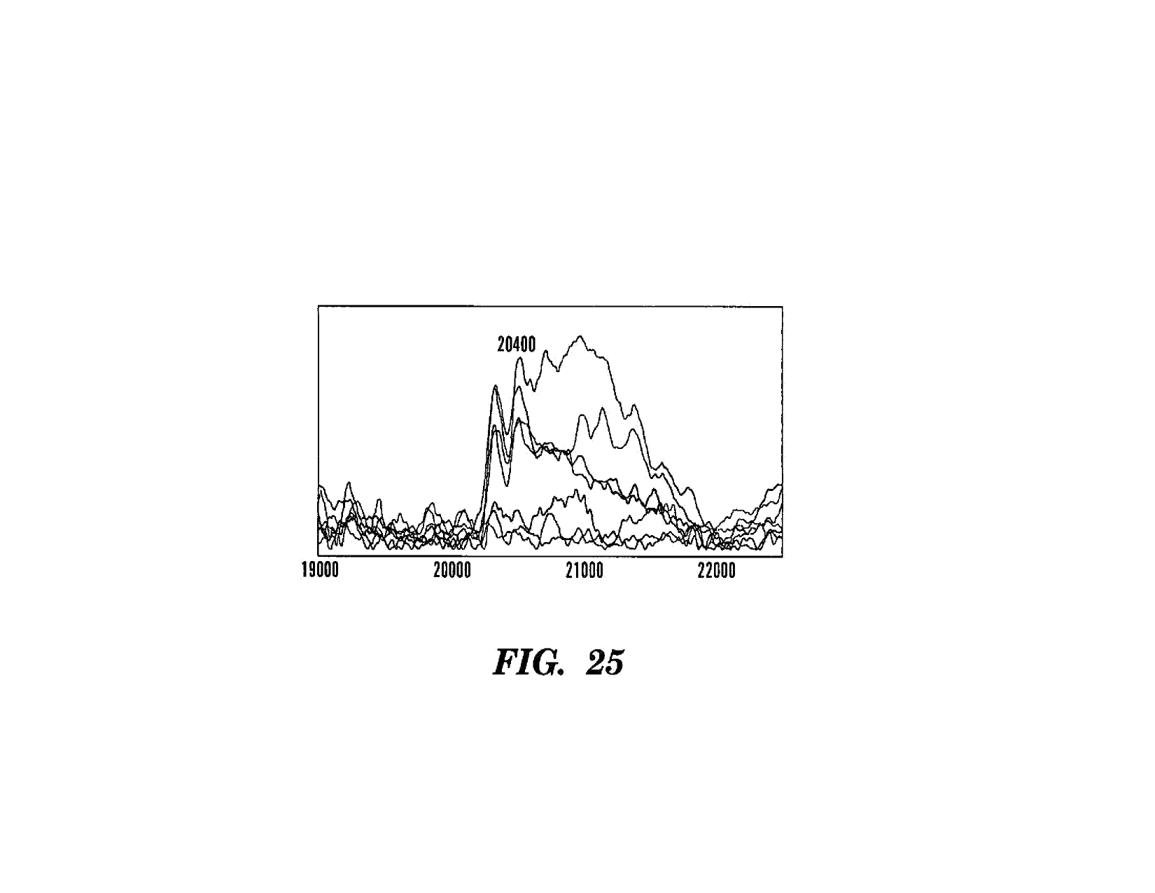

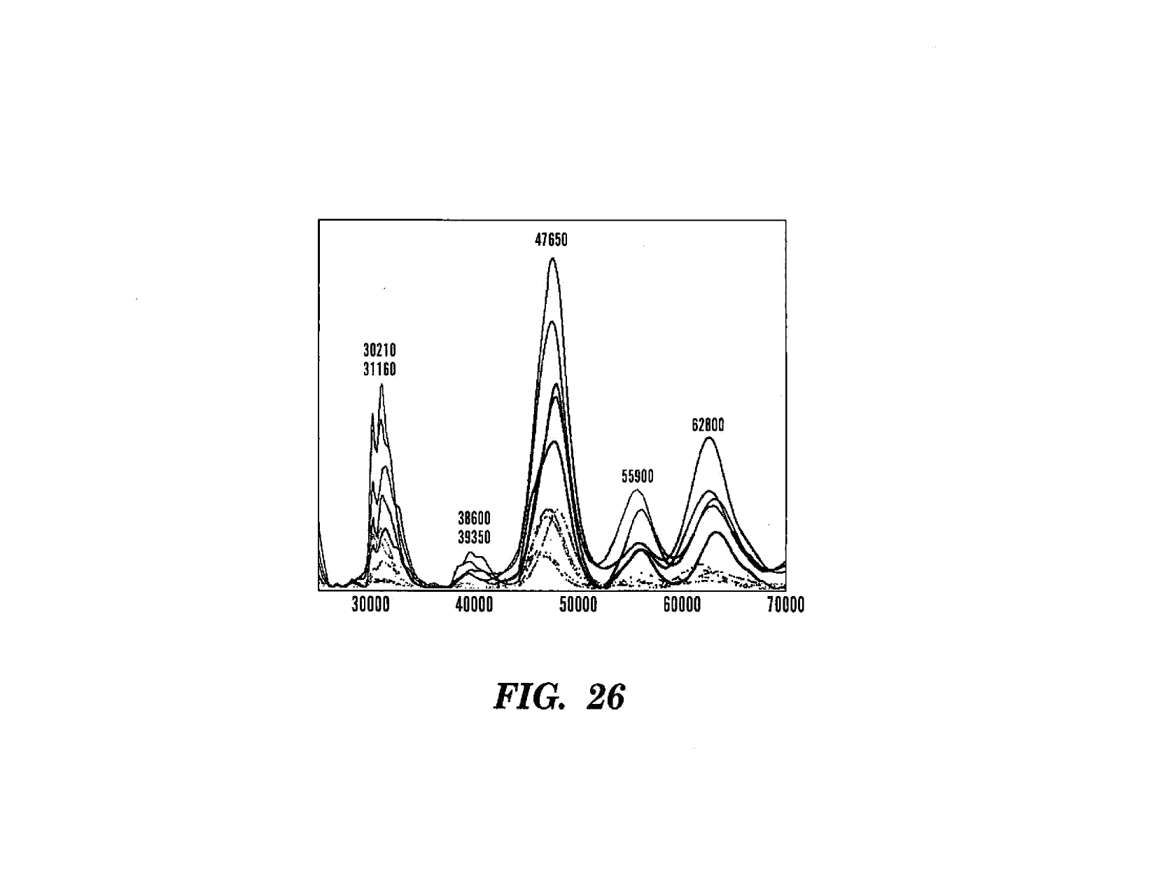

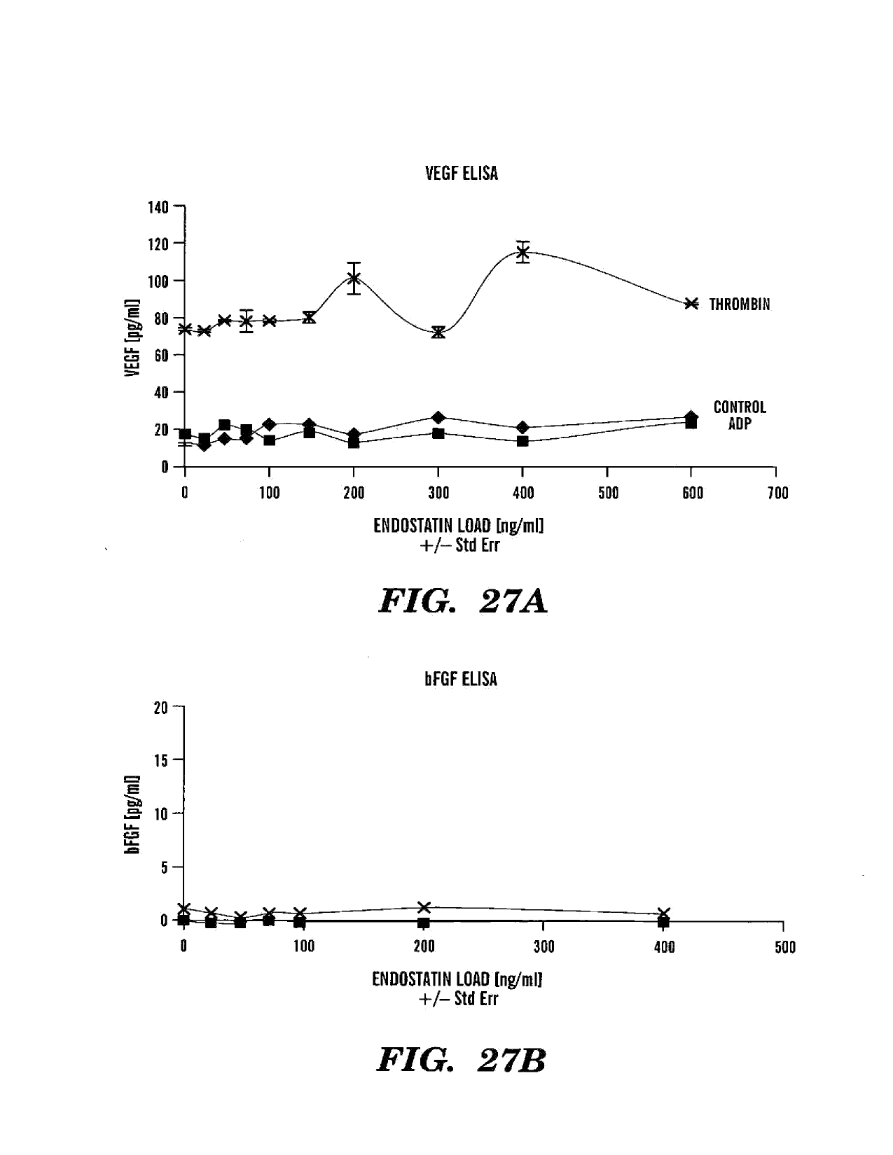

In order analyze the many factors involved in the initiation and maintenance of angiogenesis without employing preconceived or known biological systems we embarked on a proteinomic approach. In contrast to the temporally constant genome, the cell-specific protein expression is dependent on intracellular and extracellular parameters and retains significant responsiveness and variability. In the case of platelets, which, in addition to the complex protein products resulting from alternative splicing of genes in the megakaryocytes and the post translational modifications of proteins native to platelets also vary their content in response to tissue demands, we needed to employ a high through put proteome analysis such as SELDI-ToF. This technique is quickly replacing the traditional combination of two-dimensional electrophoresis followed with matrix-assisted laser desorption/ionization mass spectrometry (MALDI-MS) (17; 18), as it allows an accurate and reproducible analysis of platelet proteomes within and between experimental groups. We used an established model of non-angiogenic (dormant) and angiogenic variant of human liposarcoma SW-872 (18), in which an increase in angiogenic drive correlates with the escape from dormancy. We then compared the platelet and plasma proteomes of 5 mice injected with either 200 μl serum free media (vehicle), cell suspension of 5×106 dormant or angiogenic clone of the liposarcoma cell line. The nonangiogenic variant remains quiescent for 80-100 days, at which time 100% of the tumors begin to grow at rates comparable to the angiogenic counterpart. A comparison of platelet and plasma protein profiles at 32 days at which point the dormant tumors are on average 0.8-1 mm3 and their angiogenic complement 18-30 mm3. Two important findings emerged from the analysis. First, the plasma samples, the most commonly assayed body fluid for clinical monitoring had not revealed any significant differences in angiogenic protein profiles. Interestingly, a number of angiogenic regulators were found to be differentially expressed in platelets of tumor bearing mice. Interestingly, the selected examples of protein expression maps in FIG. 21 clearly demonstrate a similar degree of upregulation of these factors in the platelets of mice bearing the small dormant tumors. This may at first appear to be counterintuitive, but if one re-considers our previously postulated hypothesis that platelets act to enhance local angiogenesis, it may suggest that platelet sequestration of the relatively minute amounts of proteins secreted by the dormant tumor could be then protected from degradation by plasma serine proteases and facilitate efficient delivery to a site of tumor growth. A statistical analysis of expression peak intensities of sham injected controls, non-angiogenic and angiogenic tumors reveals significant differences between plasma and platelet levels of the growth factors as well as between the platelets of sham injected animals and tumor bearing animals. While the increase in VEGF and bFGF in the dormant clone never reaches statistical significance, the finding was consistent across three separate experiments and became evident as early as day 19 post tumor implantation (FIG. 23C).

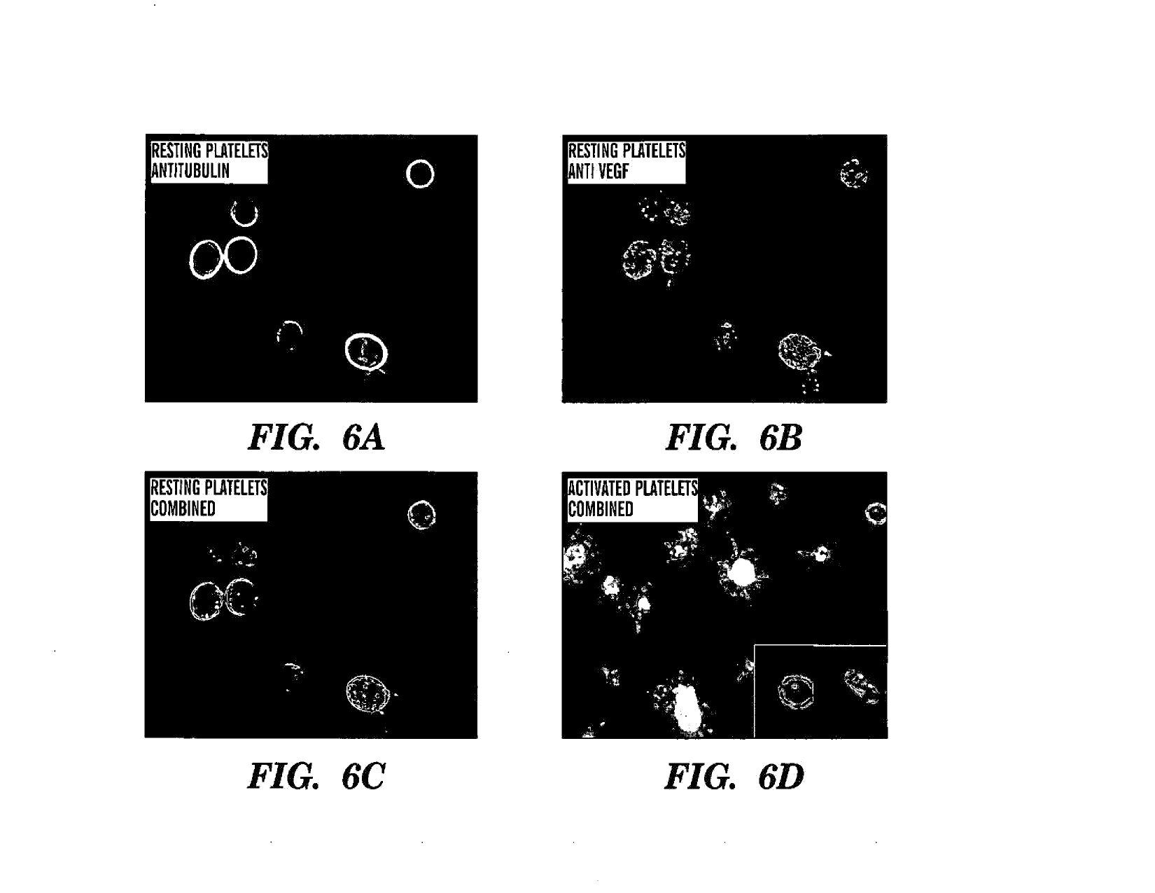

If plasma does not act as an interphase for the secretion of regulators carried by platelets an alternative mechanism may need to be postulated. We took the example of VEGF, one of the most important initiators of tumor angiogenesis and followed its intracellular distribution prior, during and post platelet activation (FIG. 6) using immunofluorescence. In resting platelet, the majority of VEGF localizes to the intracellular, cytoplasmic portion of platelets (FIG. 6 left lower panel), moving to the ring form alignment of VEGF along the cell membrane (FIG. 6 see insert in right lower panel), and then along the pseudopodia of the activated platelet (FIG. 6 right lower panel). The pattern of activation induced platelet exocytosis is more suggestive of a direct exchange of the intracellular contents of platelets with the tissues than with the commonly adopted “release” of intracellular contents of platelets into the circulation.

Furthermore, the platelet-associated angiogenic regulators appear to be “protected’ from degradation, as they persist much longer in the circulation than their plasma or platelet counterparts. For example, even though the reported half-life of VEGF in circulation is measured in minutes, the 125I-labelled VEGF picked up by platelets from the implanted Matrigel persisted in circulation for days (data not shown). This may explain why in the majority of our clinical trials, where the search for markers of early diagnosis or therapeutic response has concentrated on serum or plasma levels, has not yielded any significant advances to date. Even the use of these circulating factors for prognosis appears to be limited to the identification of subset of patients with large tumor bulk and thus poor prognosis.

Based on our study, we propose (1) Platelets uptake angiogenesis regulators directly, without a corresponding increase in plasma levels of the respective protein; (2) Platelets act to protect these regulators from degradation of serine proteases resulting in a prolongation of their half-life in circulation; and (3) Platelets can deliver these growth factors to the site of activated endothelium (tumor) without the need to raise plasma levels of these proteins. This may represent a very efficient mechanism of growth factor delivery in physiological situations such as wound healing and provide an explanation for absence of systemic side effects of these cytokines during sever stress or trauma. In the same way, this mechanism may also provide a tumor with the ability to “parasite” on the host and avoid early detection through presently available clinical tools (19).

Our data indicates that platelets are able to “detect” angiogenic regulatory protein requirement very early in cancer development and during dormancy, and that they are able to “respond” to this requirement by selective “uptake” of angiogenic regulators. It is likely that this is the mechanism by which angiogenesis regulators remain “protected” from degradation by plasma proteases, and can be “delivered” in increased concentrations to the tumor site.

### CONCLUSIONS

The flexibility and specificity of growth factor delivery by platelets has been under appreciated to date, possibly because they have been viewed as contributors rather than effectors. We have shown that the process of angiogenic regulators uptake into platelets is highly specific, reflects the tumor status (i.e dormancy vs clinical expansion) and occurs in advance of clinically detectable tumors. The identification of this novel compartment in systemic circulation, within which growth factors are protected from degradation, provides a stable, sensitive and reliable method for early cancer diagnosis. Furthermore the identification of platelets as early “register” of tumor angiogenesis suggests they should be utilized for early detection of tumor growth.

Interestingly, regulators of endothelial cell growth and/or facilitators of angiogenesis represented the majority of proteins trafficked by the platelets with limited number of other proteins being differentially expressed.

PF4, was the first chemokine to be discovered and sequenced, is platelet specific and is synthesized only in megacaryocytes. PF4 does not behave like a classic chemokine. Unlike the prototype CXC chemokine, IL-8, it does (1) not induce leucocyte chemotaxis; (2) does not cause degranulation of lysosomal granules; (3) causes a much stronger adherence to endothelium through LFA-1 then the MAC-1 facilitated IL-8 adhesion; and (4) is a selective inducer of secondary granule exocytosis in presence of TNF-α (a function not exhibited by IL-8) (Brant et al, 2000). The extremely firm neutrophil adhesion to endothelium in response to PF-4 could be system which can maintain cell-cell contact even in presence of turbulent blood flow and the induction of exocytosis subsequent to the firm adhesion could be protecting the angiogenic regulator molecules form being washed away.

CXCR chemokines are, in general, pro-angiogenic when the tripeptide ELR precedes the first CXC-domain, but anti-angiogenic when this motf is absent (Strieter R, Polyerini J B C 1995). Interestingly, the administration of full-length tetrameric (ELR-negative) PF-4 inhibits tumor growth and metastasis (Sharpe et al., J Nat Can Inst 1990 & Kolber et al., J Nat Can Instit, 1995), and its anti-angiogenic effect is due mainly to its ability to interfere with FGF-2 and VEGF binding to their respective receptors (Perollet et al., Blood 91: 3289-3299, 1998 & Gengrinovitch et al., JBC 270, 15059-15065, 1995).

One of the most differentially expressed proteins was Connective tissue activating peptide (CTAPIII, also known as low-affinity PF4). CTAPIII, neutrophil activating peptide-2 (NAP-2) and β-thrombomodulin all arise from platlet basic protein by proteolytic cleavage, which was present in high concentration in the platelets of both dormant and angiogenic liposarcoma tumor bearing mice. The identification of increased levels of this platelet associated heparanase, in both dormant as well as angiogenic clones of human liposarcomas, advocates for an important role of platelet heparanases in the early local invasion by the cancer, as well as in maintenance of tumor growth and metastatic dissemination.

Heparan sulfate, the target of CTAPIII, is an important component of the extracellular matrix and the vasculature basal lamina, which functions as a barrier to the extravasation of metastatic and inflammatory cells. Interestingly, CTAPIII functions best at pH of 5-7 (with peak optimum activity at 5.8) making it highly suitable heparanase for the relatively acidic tumor environment.

Using SELDI-ToF mass spectroscopy of platelet extracts, we have found that this novel property of platelets enables the detection of microscopic tumors that undetectable by any presently available diagnostic method. The platelet angiogenic profile is more inclusive than a single biomarker because it can detect a wide range of tumor types and tumor sizes. Relative changes in the platelet angiogenic profile permit the tracking of a tumor throughout its development, beginning from an early in situ cancer.

References cited herein are incorporated by reference.

TABLE 1

| | | |
|---|---|---|
| | Location in | |
| Angiogenic Regulators (Biomarkers) | Platelets | Reference(s) |

Stimulators of Angiogenesis

| | | |
|---|---|---|
| VEGF | α-granules | Angiogenesis. 2001; 4(1): 37-43.; Am. J. |
| | | Physiol. 1998; 275, H1054-H1061; J. |
| | | Physiol. Paris 2000; 94, 77-81; Proc. |
| | | Natl. Acad. Sci. USA 1997; 94: 663-668; |
| | | Thromb Haemost. 1998 Jul; 80(1): 171-5 |
| PDGF | α-Granules | Proc. Natl. Acad. Sci. USA 1997; 76, |
| | | 4107-4111; Endocrinology. 1989 |
| | | Apr; 124(4): 1841-8; Biochem J. 1981 Mar |
| | | 1; 193(3): 907-13 |
| bFGF | α-Granules | Blood. 1993; 82(2): 430-5; Blood. 1993 Feb |
| | | 1; 81(3): 631-8 |
| Hepatocyte growth factor (HGF) | α-Granules | Proc Natl Acad Sci USA. 1986 |
| | | Sep; 83(17): 6489-93 |
| Angiopoietin-1 | α-Granules | |
| Insulin-like growth factor (IGF)-1 and 2 | α-Granules | Blood. 1989 Aug 15; 74(3): 1084-92; |
| | | Blood. 1989 Aug 15; 74(3): 1093-1100 |
| Epidermal growth factor (EGF) | α-Granules | Am J Pathol. 1990 Oct; 137(4): 755-9.; |
| | | Regul Pept. 1992 Jan 23; 37(2): 95-100 |
| Sphingosin 1-phosphate | α-Granules | Biochem Biophys Res Commun. 1999 Nov |
| | | 2; 264(3): 743-50. Biochem Biophys Res |
| | | Commun. 1999 Nov 2; 264(3): 743-50. |
| BDNF | Unknown | Thromb Haemost. 2002 Apr; 87(4): 728-34; |
| | | Biochem Pharmacol. 1997 Jul |
| | | 1; 54(1): 207-9.; J Neurosci. 1990 |
| | | Nov; 10(11): 3469-78. |
| Thymidine Phosphorylase | α-Granules | |
| Vitronectin | α-Granules | |
| Fibronectin | α-Granules | |
| Fibrinogen | α-Granules | |
| Heparanase | α-Granules | |
| VEGFR2 | | |
| PDGFR | | |

Inhibitors of Angiogenesis

| | | |
|---|---|---|
| TSP-1 | α-Granules | FEBS Lett. 1996; 386(1): 82-6. |
| TSP-2 | | Blood. 2003 May 15; 101(10): 3915-23. |
| Endostatin | Unknown | Cell 1997; 88, 277-285; Proc Natl Acad Sci USA. |
| | | 2001; 98(11): 6470-5 |
| TGF-β1 | α-Granules | Platelets. 2003 Jun; 14(4): 233-7 |
| HGF fragments | α-Granules | Oncogene. 1998 Dec; 17 (23): 3045-3054 |
| PF-4 | α-granules | Science. 1990 Jan 5; 247(4938): 77-9 |
| Plasminogen (angiostatin) | α-Granules | |
| Plasminogen activator inhibitor | α-granules | Blood. 1996 Jun 15; 87(12): 5061-73 |
| (PAI)-1 | | |
| α-2 antiplasmin | α-Granules | Circ Res. 2000 May 12; 86(9): 952-9 |
| α-2 macroglobulin | α-Granules | J Biol Chem. 1993 Apr 15; 268(11): 7685-91; |
| | | Blood. 2001 Jun 1; 97(11): 3450-7 |
| TIMPS | α-Granules | |
| HMK domain 5 | α-Granules | |
| Fibronectin fragment | α-Granules | |
| EGF fragment | α-Granules | |
| Tumstatin | Unknown | |

TABLE 2
Additional Biomarkers

| Marker | P-Value | ProteinChip ® assay |
|---|---|---|
| 10.7, 34-39 kD | <0.05 | Fraction 1 and 2, WCX, wash |
| vascular | | with 50 mM Na acetate pH 5 |
| endothelial | | Direct on IMAC30-Cu, wash |
| growth factor | | with 50 mM TrisHCl, pH7.5 |
| (VEGF) | | |
| 20-25.7 kD | <0.05 | Fraction 1 and 2, WCX, wash |
| Platelet-derived | | with 50 mM Na acetate pH 5 |
| growth factor | | Direct on IMAC30-Cu, wash |
| (PDGF) | | with 50 mM TrisHCl, pH7.5 |
| 11, 14.7, 15, 16.5 kD | <0.05 | Fraction 1 and 2, WCX, wash |
| fibroblast | | with 50 mM Na acetate pH 5 |
| growth factor | | Direct on IMAC30-Cu, wash |
| basic (bFGF) | | with 50 mM TrisHCl, pH7.5 |
| 8206 Da platelet | <0.01 | Fraction 1 and 2, WCX, wash |
| factor 4 (PF4) | | with 50 mM Na acetate pH 5 |
| | | Direct on IMAC30-Cu, wash |
| | | with 50 mM TrisHCl, pH7.5 |
| | <0.01 | Fraction 1 and 2, WCX, wash |
| | | with 50 mM Na acetate pH 5 |
| | | Direct on CM10, wash with 50 mM |
| | | TrisHC1 pH 7.5 |
| | | Direct on IMAC30-Cu, wash |
| | | with 50 mM TrisHCl, pH7.5 |
| 13.8, 20.3 kD | <0.05 | Fraction 1 and 2, WCX, wash |
| Endostatin | | with 50 mM Na acetate pH 5 |
| | | Direct on IMAC30-Cu, wash |
| | | with 50 mM TrisHCl, pH7.5 |
| 13.8, 27.4 kD | <0.05 | Fraction 1 and 2, WCX, wash |
| Tumstatin | | with 50 mM Na acetate pH 5 |
| | | Direct on IMAC30-Cu, wash |
| | | with 50 mM TrisHC1, pH7.5 |
| 13.6, 20.6, 23.9-24.7 kD | <0.05 | Fraction 1 and 2, WCX, wash |
| Tissue inhibitor | | with 50 mM Na acetate pH 5 |
| of | | Direct on IMAC30-Cu, wash |
| metalloprotease | | with 50 mM TrisHCl, pH7.5 |
| | <0.05 | Fraction 1 and 2, WCX, wash |
| | | with 50 mM Na acetate pH 5 |
| | | Direct on IMAC30-Cu, wash |
| | | with 50 mM TrisHCl, pH7.5 |
| | | Direct on Q10, wash with 50 mM |
| | | TrisHC!, pH 7.5 |
| 8.7, 8.9 kD IL8 | <0.05 | Fraction 1 and 2, WCX, wash |
| | | with 50 mM Na acetate pH 5 |

### REFERENCE LIST

1. Pinedo, H. M., Verheul, H. M., D'Amato, R. J., and Folkman, J. 1998. Involvement of platelets in tumour angiogenesis? Lancet 352:1775-1777.
2. Banks, R. E., Forbes, M. A., Kinsey, S. E., Stanley, A., Ingham, E., Walters, C., and Selby, P. J. 1998. Release of the angiogenic cytokine vascular endothelial growth factor (VEGF) from platelets: significance for VEGF measurements and cancer biology. Br. J. Cancer 77:956-964.
3. Kim, H. K., Song, K. S., Park, Y. S., Kang, Y. H., Lee, Y. J., Lee, K. R., Kim, H. K., Ryu, K. W., Bae, J. M., and Kim, S. 2003. Elevated levels of circulating platelet microparticles, VEGF, IL-6 and RANTES in patients with gastric cancer: possible role of a metastasis predictor. Eur. J. Cancer 39:184-191.
4. Karpatkin, S. 2002. Tumor Growth and Metastasis. In Platelets. Michelson A. D., editor. Academic Press/Elsevier Sciences (USA). Amsterdam. 491-502.
5. Matsuyama, W., Hashiguchi, T., Mizoguchi, A., Iwami, F., Kawabata, M., Arimura, K., and Osame, M. 2000. Serum levels of vascular endothelial growth factor dependent on the stage progression of lung cancer. Chest 118:948-951.
6. Poon, R. T., Lau, C. P., Cheung, S. T., Yu, W. C., and Fan, S. T. 2003. Quantitative correlation of serum levels and tumor expression of vascular endothelial growth factor in patients with hepatocellular carcinoma. Cancer Res. 63:3121-3126.
7. Hlatky, L., Hahnfeldt, P., and Folkman, J. 2002. Clinical application of antiangiogenic therapy: microvessel density, what it does and doesn't tell us. J Natl. Cancer Inst. 94:883-893.
8. Karayiannakis, A. J., Bolanaki, H., Syrigos, K. N., Asimakopoulos, B., Polychronidis, A., Anagnostoulis, S., and Simopoulos, C. 2003. Serum vascular endothelial growth factor levels in pancreatic cancer patients correlate with advanced and metastatic disease and poor prognosis. Cancer Lett. 194:119-124.
9. Kim, T. K., and Burgess, D. J. 2002. Pharmacokinetic characterization of 14C-vascular endothelial growth factor controlled release microspheres using a rat model. J. Pharm. Pharmacol. 54:897-905.
10. Folkman, J. 2003. Angiogenesis and proteins of the hemostatic system. J. Thromb. Haemost. 1:1681-1682.
11. Ma, L., Elliott, S. N., Cirino, G., Buret, A., Ignarro, L. J., and Wallace, J. L. 2001. Platelets modulate gastric ulcer healing: role of endostatin and vascular endothelial growth factor release. Proc. Natl. Acad. Sci. U.S. A 98:6470-6475.
12. Nielsen, H. J., Werther, K., Mynster, T., and Brunner, N. 1999. Soluble vascular endothelial growth factor in various blood transfusion components. Transfusion 39:1078-1083.
13. Achilles, E. G., Fernandez, A., Allred, E. N., Kisker, O., Udagawa, T., Beecken, W. D., Flynn, E., and Folkman, J. 2001. Heterogeneity of angiogenic activity in a human liposarcoma: a proposed mechanism for “no take” of human tumors in mice. J. Natl. Cancer Inst. 93:1075-1081.
14. Harrison, P., Wilbourn, B., Debili, N., Vainchenker, W., Breton-Gorius, J., Lawrie, A. S., Masse, J. M., Savidge, G. F., and Cramer, E. M. 1989. Uptake of plasma fibrinogen into the alpha granules of human megakaryocytes and platelets. J. Clin. Invest 84:1320-1324.
15. Handagama, P., Scarborough, R. M., Shuman, M. A., and Bainton, D. F. 1993. Endocytosis of fibrinogen into megakaryocyte and platelet alpha-granules is mediated by alpha IIb beta 3 (glycoprotein IIb-IIIa). Blood 82:135-138.
16. Handagama, P. J., George, J. N., Shuman, M. A., McEver, R. P., and Bainton, D. F. 1987. Incorporation of a circulating protein into megakaryocyte and platelet granules. Proc. Natl. Acad. Sci. U.S. A 84:861-865.
17. Marcus, K., Immler, D., Sternberger, J., and Meyer, H. E. 2000. Identification of platelet proteins separated by two-dimensional gel electrophoresis and analyzed by matrix assisted laser desorption/ionization-time of flight-mass spectrometry and detection of tyrosine-phosphorylated proteins. Electrophoresis 21:2622-2636.
18. O'Neill, E. E., Brock, C. J., von Kriegsheim, A. F., Pearce, A. C., Dwek, R. A., Watson, S. P., and Hebestreit, H. F. 2002. Towards complete analysis of the platelet proteome. Proteomics. 2:288-305.
19. Broll, R., Erdmann, H., Duchrow, M., Oevermann, E., Schwandner, O., Markert, U., Bruch, H. P., and Windhovel, U. 2001. Vascular endothelial growth factor (VEGF)—a valuable serum tumour marker in patients with colorectal cancer? Eur. J. Surg. Oncol. 27:37-42.
20. Udagawa, T., Fernandez, A., Achilles, E. G., Folkman, J., & D'Amato, R. J. Persistence of microscopic human cancers in mice: alterations in the angiogenic balance accompanies loss of tumor dormancy. FASEB J 16, 1361-1370 (2002).
21. Gimbrone, M. A., Jr., Leapman, S. B., Cotran, R. S., & Folkman, J. Tumor dormancy in vivo by prevention of neovascularization. J. Exp. Med. 136, 261-276 (1972).
22. Holmgren, L., O′ reilly, M. S., & Folkman, J. Dormancy of micrometastases: balanced proliferation and apoptosis in the presence of angiogenesis suppression [see comments]. Nat. Med. 1, 149-153 (1995).
23. Roy, R., Wewer, U. M., Zurakowski, D., Pories, S. E., & Moses, M. A. ADAM 12 cleaves extracellular matrix proteins and correlates with cancer status and stage. J. Biol. Chem. 279, 51323-51330 (2004).
24. Folkman, J. & Kalluri, R. Cancer without disease. Nature 427, 787 (2004).

References cited herein are incorporated by reference.

1. A method for the detection of an angiogenic disease or disorder in an individual comprising the steps of:
a. isolating platelets from said individual at a first time point;
b. analyzing said platelets for the level of at least one positive or at least one negative angiogenic regulator;
c. isolating platelets from said individual at a second time point, said second time point being after said first time point;
d. analyzing said platelets from said second time point for the level of at least one positive or at least one negative angiogenic regulator; and
e. comparing the levels of said angiogenic regulator from the first time point to the levels of said angiogenic regulator from said second time point, wherein an increase in the level of said at least one positive angiogenic regulator in the platelets from said second time point or a decrease in at least one negative angiogenic regulator in the platelets from said second time point is indicative of an angiogenic disease or disorder.
